SEQUENCE LISTING (133 sequences)

SEQ ID NO: 1 - protein, 5 aa, Artificial Sequence
  source: /note=“Description of Artificial Sequence Synthetic peptide”
TFWMH

SEQ ID NO: 2 - protein, 17 aa, Artificial Sequence
  source: /note=“Description of Artificial Sequence Synthetic peptide”
NIYPGSGTINYDEKFRS

SEQ ID NO: 3 - protein, 6 aa, Artificial Sequence
  source: /note=“Description of Artificial Sequence Synthetic peptide”
GWDGEH

SEQ ID NO: 4 - protein, 5 aa, Artificial Sequence
  source: /note=“Description of Artificial Sequence Synthetic peptide”
DYGMH

SEQ ID NO: 5 - protein, 17 aa, Artificial Sequence
  source: /note=“Description of Artificial Sequence Synthetic peptide”
YIGTTSSIIYYADTVKG

SEQ ID NO: 6 - protein, 12 aa, Artificial Sequence
  source: /note=“Description of Artificial Sequence Synthetic peptide”
RDYGNYYWYLDV

SEQ ID NO: 7 - protein, 11 aa, Artificial Sequence
  source: /note=“Description of Artificial Sequence Synthetic peptide”
RASENIHSNLA

SEQ ID NO: 8 - protein, 7 aa, Artificial Sequence
  source: /note=“Description of Artificial Sequence Synthetic peptide”
GATNLAD

SEQ ID NO: 9 - protein, 10 aa, Artificial Sequence
  source: /note=“Description of Artificial Sequence Synthetic peptide”
QHFWGTPPYA

SEQ ID NO: 10 - protein, 10 aa, Artificial Sequence
  source: /note=“Description of Artificial Sequence Synthetic peptide”
SASSSVFDMY

SEQ ID NO: 11 - protein, 7 aa, Artificial Sequence
  source: /note=“Description of Artificial Sequence Synthetic peptide”
RTSNLAS

SEQ ID NO: 12 - protein, 9 aa, Artificial Sequence
  source: /note=“Description of Artificial Sequence Synthetic peptide”
QQYQSFPLT

SEQ ID NO: 13 - protein, 5 aa, Artificial Sequence
  source: /note=“Description of Artificial Sequence Synthetic peptide”
FYTMH

SEQ ID NO: 14 - protein, 17 aa, Artificial Sequence
  source: /note=“Description of Artificial Sequence Synthetic peptide”
YINPSSGYTNYNQKFTD

SEQ ID NO: 15 - protein, 12 aa, Artificial Sequence
  source: /note=“Description of Artificial Sequence Synthetic peptide”
SDGSSSKWYFDV

SEQ ID NO: 16 - protein, 5 aa, Artificial Sequence
  source: /note=“Description of Artificial Sequence Synthetic peptide”
DYYIH

SEQ ID NO: 17 - protein, 17 aa, Artificial Sequence
  source: /note=“Description of Artificial Sequence Synthetic peptide”
RIDPEDGDIAYAPKFQD

SEQ ID NO: 18 - protein, 8 aa, Artificial Sequence
  source: /note=“Description of Artificial Sequence Synthetic peptide”
GNYYAMDF

SEQ ID NO: 19 - protein, 10 aa, Artificial Sequence
  source: /note=“Description of Artificial Sequence Synthetic peptide”
RASSSVSYIH

SEQ ID NO: 20 - protein, 7 aa, Artificial Sequence
  source: /note=“Description of Artificial Sequence Synthetic peptide”
ATSNLAS

SEQ ID NO: 21 - protein, 9 aa, Artificial Sequence
  source: /note=“Description of Artificial Sequence Synthetic peptide”
QQWSSNPFT

SEQ ID NO: 22 - protein, 12 aa, Artificial Sequence
  source: /note=“Description of Artificial Sequence Synthetic peptide”
TASSSVSSSYLH

SEQ ID NO: 23 - protein, 7 aa, Artificial Sequence
  source: /note=“Description of Artificial Sequence Synthetic peptide”
STSNLAS

SEQ ID NO: 24 - protein, 9 aa, Artificial Sequence
  source: /note=“Description of Artificial Sequence Synthetic peptide”
HQYHRSPPT

SEQ ID NO: 25 - protein, 115 aa, Artificial Sequence
  source: /note=“Description of Artificial Sequence Synthetic polypeptide”
QVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSS

SEQ ID NO: 26 - protein, 121 aa, Artificial Sequence
  source: /note=“Description of Artificial Sequence Synthetic polypeptide”
EVQLVESGGGLVKPGGSLRLSCAASGFTFSDYGMHWIRQAPGKGLEWIAYIGTTSSIIYYADTVKGRFTISRDNAKNSLYLQMNSLRAEDTAVYYCARRDYGNYYWYLDVWGTGTMVTVSS

SEQ ID NO: 27 - protein, 108 aa, Artificial Sequence
  source: /note=“Description of Artificial Sequence Synthetic polypeptide”
DIQMTQSPSSLSVSVGDRVTITCRASENIHSNLAWYQQKPGKAPQLLVYGATNLADGVPSRFSGSGSGAQYTLTISSLQPEDFATYYCQHFWGTPPYAFGGGTKLEIK

SEQ ID NO: 28 - protein, 106 aa, Artificial Sequence
  source: /note=“Description of Artificial Sequence Synthetic polypeptide”
EIVLTQSPATLSLSPGERVTLSCSASSSVFDMYWYQQKPGSSPRPWIYRTSNLASGVPARFSGSGSGTDFFLTISSLEPEDAAVYYCQQYQSFPLTFGQGTKLELK

SEQ ID NO: 29 - protein, 121 aa, Artificial Sequence
  source: /note=“Description of Artificial Sequence Synthetic polypeptide”
QVQLVQSGAEVKKPGASVKMSCKASGYTFTFYTMHWLKQAPGQGLEWIGYINPSSGYTNYNQKFTDRATLTADKSTSTAYMELSSLRSEDTAVYYCARSDGSSSKWYFDVWGQGTTVTVSS

SEQ ID NO: 30 - protein, 117 aa, Artificial Sequence
  source: /note=“Description of Artificial Sequence Synthetic polypeptide”
EVQLVQSGAEVKKPGATVKISCKASGFNIKDYYIHWVNQAPGKGLEWIGRIDPEDGDIAYAPKFQDRVTLTVDTSTDTAYLELSSLRSEDTAVYYCTTGNYYAMDFWGQGTTVTVSS

SEQ ID NO: 31 - protein, 106 aa, Artificial Sequence
  source: /note=“Description of Artificial Sequence Synthetic polypeptide”
EIVLTQSPDFQSATPKEKVTITCRASSSVSYIHWYQQKPGSSPKAWISATSNLASGVPSRFSGSGSGTSYTLTINRVEAEDAATYYCQQWSSNPFTFGQGTKLEIK

SEQ ID NO: 32 - protein, 108 aa, Artificial Sequence
  source: /note=“Description of Artificial Sequence Synthetic polypeptide”
QIVLTQSPATLSASPGERVTLSCTASSSVSSSYLHWYQQKPGSSPKLWIYSTSNLASGVPARFSGSGPGTSYTLTISSMEPEDAATYYCHQYHRSPPTFGQGTKLEIK

SEQ ID NO: 33 - protein, 107 aa, Homo sapiens [sequence omitted]

SEQ ID NO: 34 - protein, 330 aa, Homo sapiens [sequence omitted]

SEQ ID NO: 35 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 5 aa, Artificial Sequence
  source: /note=“Description of Artificial Sequence Synthetic peptide”
GGGGS

SEQ ID NO: 37 - protein, 10 aa, Artificial Sequence
  source: /note=“Description of Artificial Sequence Synthetic peptide”
GGGGSGGGGS

SEQ ID NO: 38 - protein, 15 aa, Artificial Sequence
  source: /note=“Description of Artificial Sequence Synthetic peptide”
GGGGSGGGGSGGGGS

SEQ ID NO: 39 - protein, 22 aa, Artificial Sequence
  source: /note=“Description of Artificial Sequence Synthetic peptide”
MDMRVPAQLLGLLLLWLRGARC

SEQ ID NO: 40 - protein, 20 aa, Artificial Sequence
  source: /note=“Description of Artificial Sequence Synthetic peptide”
MRVPAQLLGLLLLWFPGARC

SEQ ID NO: 41 - protein, 713 aa, Artificial Sequence
  source: /note=“Description of Artificial Sequence Synthetic polypeptide”
MDMRVPAQLLGLLLLWLRGARCQVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSQVQLVQSGAEVKKPGASVKMSCKASGYTFTFYTMHWLKQAPGQCLEWIGYINPSSGYTNYNQKFTDRATLTADKSTSTAYMELSSLRSEDTAVYYCARSDGSSSKWYFDVWGQGTTVTVSSGGGGSGGGGSGGGGSEIVLTQSPDFQSATPKEKVTITCRASSSVSYIHWYQQKPGSSPKAWISATSNLASGVPSRFSGSGSGTSYTLTINRVEAEDAATYYCQQWSSNPFTFGCGTKLEIK

SEQ ID NO: 42 - protein, 691 aa, Artificial Sequence
  source: /note=“Description of Artificial Sequence Synthetic polypeptide”
QVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSQVQLVQSGAEVKKPGASVKMSCKASGYTFTFYTMHWLKQAPGQCLEWIGYINPSSGYTNYNQKFTDRATLTADKSTSTAYMELSSLRSEDTAVYYCARSDGSSSKWYFDVWGQGTTVTVSSGGGGSGGGGSGGGGSEIVLTQSPDFQSATPKEKVTITCRASSSVSYIHWYQQKPGSSPKAWISATSNLASGVPSRFSGSGSGTSYTLTINRVEAEDAATYYCQQWSSNPFTFGCGTKLEIK

SEQ ID NO: 43 - protein, 235 aa, Artificial Sequence
  source: /note=“Description of Artificial Sequence Synthetic polypeptide”
MRVPAQLLGLLLLWFPGARCDIQMTQSPSSLSVSVGDRVTITCRASENIHSNLAWYQQKPGKAPQLLVYGATNLADGVPSRFSGSGSGAQYTLTISSLQPEDFATYYCQHFWGTPPYAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 215 aa, Artificial Sequence
  source: /note=“Description of Artificial Sequence Synthetic polypeptide”
DIQMTQSPSSLSVSVGDRVTITCRASENIHSNLAWYQQKPGKAPQLLVYGATNLADGVPSRFSGSGSGAQYTLTISSLQPEDFATYYCQHFWGTPPYAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 121 aa, Artificial Sequence
  source: /note=“Description of Artificial Sequence Synthetic polypeptide”
QVQLVQSGAEVKKPGASVKMSCKASGYTFTFYTMHWLKQAPGQCLEWIGYINPSSGYTNYNQKFTDRATLTADKSTSTAYMELSSLRSEDTAVYYCARSDGSSSKWYFDVWGQGTTVTVSS

SEQ ID NO: 46 - protein, 117 aa, Artificial Sequence
  source: /note=“Description of Artificial Sequence Synthetic polypeptide”
EVQLVQSGAEVKKPGATVKISCKASGFNIKDYYIHWVNQAPGKCLEWIGRIDPEDGDIAYAPKFQDRVTLTVDTSTDTAYLELSSLRSEDTAVYYCTTGNYYAMDFWGQGTTVTVSS

SEQ ID NO: 47 - protein, 106 aa, Artificial Sequence
  source: /note=“Description of Artificial Sequence Synthetic polypeptide”
EIVLTQSPDFQSATPKEKVTITCRASSSVSYIHWYQQKPGSSPKAWISATSNLASGVPSRFSGSGSGTSYTLTINRVEAEDAATYYCQQWSSNPFTFGCGTKLEIK

SEQ ID NO: 48 - protein, 108 aa, Artificial Sequence
  source: /note=“Description of Artificial Sequence Synthetic polypeptide”
QIVLTQSPATLSASPGERVTLSCTASSSVSSSYLHWYQQKPGSSPKLWIYSTSNLASGVPARFSGSGPGTSYTLTISSMEPEDAATYYCHQYHRSPPTFGCGTKLEIK

SEQ ID NO: 49 - protein, 16 aa, Artificial Sequence
  source: /note=“Description of Artificial Sequence Synthetic peptide”
GGGGSGGGGSGGGGSA

SEQ ID NO: 50 - protein, 5 aa, Artificial Sequence
  source: /note=“Description of Artificial Sequence Synthetic peptide”
GYYMN

SEQ ID NO: 51 - protein, 17 aa, Artificial Sequence
  source: /note=“Description of Artificial Sequence Synthetic peptide”
DLNPDNGDTNYNQKFVG

SEQ ID NO: 52 - protein, 8 aa, Artificial Sequence
  source: /note=“Description of Artificial Sequence Synthetic peptide”
GGKGGFDY

SEQ ID NO: 53 - protein, 5 aa, Artificial Sequence
  source: /note=“Description of Artificial Sequence Synthetic peptide”
DYYIN

SEQ ID NO: 54 - protein, 17 aa, Artificial Sequence
  source: /note=“Description of Artificial Sequence Synthetic peptide”
WIYPGSGNTKYNEKFKD

SEQ ID NO: 55 - protein, 15 aa, Artificial Sequence
  source: /note=“Description of Artificial Sequence Synthetic peptide”
KGIIYNYGSSDVLAY

SEQ ID NO: 56 - protein, 16 aa, Artificial Sequence
  source: /note=“Description of Artificial Sequence Synthetic peptide”
RSSQSLEKSNGNTYLN

SEQ ID NO: 57 - protein, 7 aa, Artificial Sequence
  source: /note=“Description of Artificial Sequence Synthetic peptide”
RVSRRYS

SEQ ID NO: 58 - protein, 9 aa, Artificial Sequence
  source: /note=“Description of Artificial Sequence Synthetic peptide”
LQVTHVPYT

SEQ ID NO: 59 - protein, 17 aa, Artificial Sequence
  source: /note=“Description of Artificial Sequence Synthetic peptide”
KSSQSLLYSSNQKNYLA

SEQ ID NO: 60 - protein, 7 aa, Artificial Sequence
  source: /note=“Description of Artificial Sequence Synthetic peptide”
WASTRES

SEQ ID NO: 61 - protein, 9 aa, Artificial Sequence
  source: /note=“Description of Artificial Sequence Synthetic peptide”
HQYYSYPLT

SEQ ID NO: 62 - protein, 117 aa, Artificial Sequence
  source: /note=“Description of Artificial Sequence Synthetic polypeptide”
EVQLQQSGAEVKKPGASVKISCKASGYTFTGYYMNWMKQSHGKSLEWIGDLNPDNGDTNYNQKFVGRATLTVDKSISTAYMELSRLRSEDTAVYYCARGGKGGFDYWGQGTTLTVSS

SEQ ID NO: 63 - protein, 124 aa, Artificial Sequence
  source: /note=“Description of Artificial Sequence Synthetic polypeptide”
QIQLQQSGAEVKKPGASVKISCKASGYTFIDYYINWVKQRPGQGLEWIGWIYPGSGNTKYNEKFKDRGTLTVDTSSSTAYMELSSLRSEDTAVYFCVRKGIIYNYGSSDVLAYWGQGTLVTVSS

SEQ ID NO: 64 - protein, 112 aa, Artificial Sequence
  source: /note=“Description of Artificial Sequence Synthetic polypeptide”
DAVMTQSPLSLPVTLGQPASISCRSSQSLEKSNGNTYLNWYLQRPGQSPQLLIYRVSRRYSGVPDRFSGSGSGTDFTLKISRVEAEDVGVYYCLQVTHVPYTFGQGTRLEIK

SEQ ID NO: 65 - protein, 113 aa, Artificial Sequence
  source: /note=“Description of Artificial Sequence Synthetic polypeptide”
DIVMTQSPDSLAVSLGERLTINCKSSQSLLYSSNQKNYLAWYQQKPGQSPKLLIYWASTRESGVPDRFSGSGSGTDFTLTISSVQAEDVAVYYCHQYYSYPLTFGQGTKLELK

SEQ ID NO: 66 - protein, 215 aa, Artificial Sequence; identical to SEQ ID NO: 44
  source: /note=“Description of Artificial Sequence Synthetic polypeptide”

SEQ ID NO: 67 - protein, 441 aa, Artificial Sequence
  source: /note=“Description of Artificial Sequence Synthetic polypeptide”
QVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 68 - protein, 473 aa, Artificial Sequence
  source: /note=“Description of Artificial Sequence Synthetic polypeptide”
EVQLQQSGAEVKKPGASVKISCKASGYTFTGYYMNWMKQSHGKCLEWIGDLNPDNGDTNYNQKFVGRATLTVDKSISTAYMELSRLRSEDTAVYYCARGGKGGFDYWGQGTTLTVSSGGGGSGGGGSGGGGSADVVMTQSPLSLPVTLGQPASISCRSSQSLEKSNGNTYLNWYLQRPGQSPQLLIYRVSRRYSGVPDRFSGSGSGTDFTLKISRVEAEDVGVYYCLQVTHVPYTFGCGTRLEIKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 69 - protein, 215 aa, Artificial Sequence; identical to SEQ ID NO: 44
  source: /note=“Description of Artificial Sequence Synthetic polypeptide”

SEQ ID NO: 70 - protein, 441 aa, Artificial Sequence; identical to SEQ ID NO: 67
  source: /note=“Description of Artificial Sequence Synthetic polypeptide”

SEQ ID NO: 71 - protein, 481 aa, Artificial Sequence
  source: /note=“Description of Artificial Sequence Synthetic polypeptide”
QIQLQQSGAEVKKPGASVKISCKASGYTFIDYYINWVKQRPGQCLEWIGWIYPGSGNTKYNEKFKDRGTLTVDTSSSTAYMELSSLRSEDTAVYFCVRKGIIYNYGSSDVLAYWGQGTLVTVSSGGGGSGGGGSGGGGSADIVMTQSPDSLAVSLGERLTINCKSSQSLLYSSNQKNYLAWYQQKPGQSPKLLIYWASTRESGVPDRFSGSGSGTDFTLTISSVQAEDVAVYYCHQYYSYPLTFGCGTKLELKESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 72 - protein, 245 aa, Artificial Sequence
  source: /note=“Description of Artificial Sequence Synthetic polypeptide”
EVQLQQSGAEVKKPGASVKISCKASGYTFTGYYMNWMKQSHGKCLEWIGDLNPDNGDTNYNQKFVGRATLTVDKSISTAYMELSRLRSEDTAVYYCARGGKGGFDYWGQGTTLTVSSGGGGSGGGGSGGGGSADVVMTQSPLSLPVTLGQPASISCRSSQSLEKSNGNTYLNWYLQRPGQSPQLLIYRVSRRYSGVPDRFSGSGSGTDFTLKISRVEAEDVGVYYCLQVTHVPYTFGCGTRLEIK

SEQ ID NO: 73 - protein, 245 aa, Artificial Sequence
  source: /note=“Description of Artificial Sequence Synthetic polypeptide”
EVQLQQSGAEVKKPGASVKISCKASGYTFTGYYMNWMKQSHGKSLEWIGDLNPDNGDTNYNQKFVGRATLTVDKSISTAYMELSRLRSEDTAVYYCARGGKGGFDYWGQGTTLTVSSGGGGSGGGGSGGGGSADVVMTQSPLSLPVTLGQPASISCRSSQSLEKSNGNTYLNWYLQRPGQSPQLLIYRVSRRYSGVPDRFSGSGSGTDFTLKISRVEAEDVGVYYCLQVTHVPYTFGQGTRLEIK

SEQ ID NO: 74 - protein, 253 aa, Artificial Sequence
  source: /note=“Description of Artificial Sequence Synthetic polypeptide”
QIQLQQSGAEVKKPGASVKISCKASGYTFIDYYINWVKQRPGQCLEWIGWIYPGSGNTKYNEKFKDRGTLTVDTSSSTAYMELSSLRSEDTAVYFCVRKGIIYNYGSSDVLAYWGQGTLVTVSSGGGGSGGGGSGGGGSADIVMTQSPDSLAVSLGERLTINCKSSQSLLYSSNQKNYLAWYQQKPGQSPKLLIYWASTRESGVPDRFSGSGSGTDFTLTISSVQAEDVAVYYCHQYYSYPLTFGCGTKLELK

SEQ ID NO: 75 - protein, 253 aa, Artificial Sequence
  source: /note=“Description of Artificial Sequence Synthetic polypeptide”
QIQLQQSGAEVKKPGASVKISCKASGYTFIDYYINWVKQRPGQGLEWIGWIYPGSGNTKYNEKFKDRGTLTVDTSSSTAYMELSSLRSEDTAVYFCVRKGIIYNYGSSDVLAYWGQGTLVTVSSGGGGSGGGGSGGGGSADIVMTQSPDSLAVSLGERLTINCKSSQSLLYSSNQKNYLAWYQQKPGQSPKLLIYWASTRESGVPDRFSGSGSGTDFTLTISSVQAEDVAVYYCHQYYSYPLTFGQGTKLELK

SEQ ID NO: 76 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 77 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 78 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 79 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 80 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLG

SEQ ID NO: 81 - protein, 228 aa, Artificial Sequence
  source: /note=“Description of Artificial Sequence Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 82 - protein, 228 aa, Artificial Sequence
  source: /note=“Description of Artificial Sequence Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 83 - protein, 228 aa, Artificial Sequence
  source: /note=“Description of Artificial Sequence Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 84 - protein, 228 aa, Artificial Sequence
  source: /note=“Description of Artificial Sequence Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNRFTQKSLSLSLG

SEQ ID NO: 85 - protein, 228 aa, Artificial Sequence
  source: /note=“Description of Artificial Sequence Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNRFTQKSLSLSLG

SEQ ID NO: 86 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 87 - protein, 175 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTT

SEQ ID NO: 88 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 89 - protein, 214 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 90 - protein, 103 aa, Homo sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFEN

SEQ ID NO: 91 - protein, 107 aa, Artificial Sequence [sequence omitted]

SEQ ID NO: 92 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 329 aa, Artificial Sequence; identical to SEQ ID NO: 35
  source: /note=“Description of Artificial Sequence Synthetic polypeptide”

SEQ ID NO: 94 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCLAKGFYPSDIAVEWESNGQPENNYKTTPPVLDSDGSFFLVSKLTVDKSRWQQGNVFSCSVMHEALHNHYTQKSLSLSPG

SEQ ID NO: 96 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 97 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCLAKGFYPSDIAVEWESNGQPENNYKTTPPVLDSDGSFFLVSKLTVDKSRWQQGNVFSCSVMHEALHNHYTQKSLSLSPG

SEQ ID NO: 98 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCLAKGFYPSDIAVEWESNGQPENNYKTTPPVLDSDGSFFLVSKLTVDKSRWQQGNVFSCSVMHEALHNHYTQKSLSLSPG

SEQ ID NO: 100 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 101 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLSCLAKGFYPSDIAVEWESNGQPENNYKTTPPVLDSDGSFFLVSKLTVDKSRWQQGNVFSCSVMHEALHNRFTQKSLSLSPG

SEQ ID NO: 102 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPG

SEQ ID NO: 103 - protein, 329 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCLAKGFYPSDIAVEWESNGQPENNYKTTPPVLDSDGSFFLVSKLTVDKSRWQQGNVFSCSVMHEALHNRFTQKSLSLSPG

SEQ ID NO: 104 - protein, 215 aa, Artificial Sequence; identical to SEQ ID NO: 44
  source: /note=“Description of Artificial Sequence Synthetic polypeptide”

SEQ ID NO: 105 - protein, 658 aa, Artificial Sequence
  source: /note=“Description of Artificial Sequence Synthetic polypeptide”
QVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 106 - protein, 215 aa, Artificial Sequence; identical to SEQ ID NO: 44
  source: /note=“Description of Artificial Sequence Synthetic polypeptide”

SEQ ID NO: 107 - protein, 658 aa, Artificial Sequence
  source: /note=“Description of Artificial Sequence Synthetic polypeptide”
QVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGGGGSGGGGSITCPPPMSVEHADIWVKSYSLYSRERYICNSGFKRKAGTSSLTECVLNKATNVAHWTTPSLKCIR

SEQ ID NO: 108 - protein, 215 aa, Artificial Sequence; identical to SEQ ID NO: 44
  source: /note=“Description of Artificial Sequence Synthetic polypeptide”

SEQ ID NO: 109 - protein, 583 aa, Artificial Sequence
  source: /note=“Description of Artificial Sequence Synthetic polypeptide”
QVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCLAKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 110 - protein, 534 aa, Artificial Sequence
  source: /note=“Description of Artificial Sequence Synthetic polypeptide”
QVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSGGGGSITCPPPMSVEHADIWVKSYSLYSRERYICNSGFKRKAGTSSLTECVLNKATNVAHWTTPSLKCIR

SEQ ID NO: 111 - protein, 215 aa, Artificial Sequence; identical to SEQ ID NO: 44
  source: /note=“Description of Artificial Sequence Synthetic polypeptide”

SEQ ID NO: 112 - protein, 658 aa, Artificial Sequence
  source: /note=“Description of Artificial Sequence Synthetic polypeptide”
QVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSITCPPPMSVEHADIWVKSYSLYSRERYICNSGFKRKAGTSSLTECVLNKATNVAHWTTPSLKCIRGGGGSGGGGSGGGGSGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 113 - protein, 444 aa, Artificial Sequence
  source: /note=“Description of Artificial Sequence Synthetic polypeptide”
QVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCLAKGFYPSDIAVEWESNGQPENNYKTTPPVLDSDGSFFLVSKLTVDKSRWQQGNVFSCSVMHEALHNHYTQKSLSLSPG

SEQ ID NO: 114 - protein, 215 aa, Artificial Sequence; identical to SEQ ID NO: 44
  source: /note=“Description of Artificial Sequence Synthetic polypeptide”

SEQ ID NO: 115 - protein, 668 aa, Artificial Sequence
  source: /note=“Description of Artificial Sequence Synthetic polypeptide”
QVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSGGGGSGGGGSGGGGSGGGGSGGGGSGGGGSITCPPPMSVEHADIWVKSYSLYSRERYICNSGFKRKAGTSSLTECVLNKATNVAHWTTPSLKCIR

SEQ ID NO: 116 - protein, 444 aa, Artificial Sequence; identical to SEQ ID NO: 113
  source: /note=“Description of Artificial Sequence Synthetic polypeptide”

SEQ ID NO: 117 - protein, 215 aa, Artificial Sequence; identical to SEQ ID NO: 44
  source: /note=“Description of Artificial Sequence Synthetic polypeptide”

SEQ ID NO: 118 - protein, 658 aa, Artificial Sequence; identical to SEQ ID NO: 112
  source: /note=“Description of Artificial Sequence Synthetic polypeptide”

SEQ ID NO: 119 - protein, 687 aa, Artificial Sequence
  source: /note=“Description of Artificial Sequence Synthetic polypeptide”
QVQLVQSGAEVVKPGASVKLSCKASGYTFTTFWMHWVRQAPGQGLEWIGNIYPGSGTINYDEKFRSRATLTVDTSISTAYMEVSRLRSEDTAVYYCTTGWDGEHWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCLAKGFYPSDIAVEWESNGQPENNYKTTPPVLDSDGSFFLVSKLTVDKSRWQQGNVFSCSVMHEALHNHYTQKSLSLSPGGSSGRIGFLRTAGSLGGSGRSANAILEGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 120 - protein, 12 aa, Artificial Sequence
  source: /note=“Description of Artificial Sequence Synthetic peptide”
GGSGGSGGSGGS

SEQ ID NO: 121 - protein, 20 aa, Artificial Sequence
  source: /note=“Description of Artificial Sequence Synthetic peptide”
GGGGSGGGGSGGGGSGGGGS

SEQ ID NO: 122 - protein, 25 aa, Artificial Sequence
  source: /note=“Description of Artificial Sequence Synthetic peptide”
GGGGSGGGGSGGGGSGGGGSGGGGS

SEQ ID NO: 123 - protein, 30 aa, Artificial Sequence
  source: /note=“Description of Artificial Sequence Synthetic polypeptide”
GGGGSGGGGSGGGGSGGGGSGGGGSGGGGS

SEQ ID NO: 124 - protein, 35 aa, Artificial Sequence
  source: /note=“Description of Artificial Sequence Synthetic polypeptide”
GGGGSGGGGSGGGGSGGGGSGGGGSGGGGSGGGGS

SEQ ID NO: 125 - protein, 29 aa, Artificial Sequence
  source: /note=“Description of Artificial Sequence Synthetic peptide”
GSSGRIGFLRTAGSLGGSGRSANAILEGS

SEQ ID NO: 126 - protein, 29 aa, Artificial Sequence
  source: /note=“Description of Artificial Sequence Synthetic peptide”
GSLGGSGRSANAILEGSSGRIGFLRTAGS

SEQ ID NO: 127 - protein, 38 aa, Artificial Sequence
  source: /note=“Description of Artificial Sequence Synthetic polypeptide”
GGGGSSGRIGFLRTAGGGGSLGGSGRSANAILEGGGGS

SEQ ID NO: 128 - protein, 38 aa, Artificial Sequence
  source: /note=“Description of Artificial Sequence Synthetic polypeptide”
GGGGSLGGSGRSANAILEGGGGSSGRIGFLRTAGGGGS

SEQ ID NO: 129 - protein, 242 aa, Artificial Sequence
  source: /note=“Description of Artificial Sequence Synthetic polypeptide”
QVQLVQSGAEVKKPGASVKMSCKASGYTFTFYTMHWLKQAPGQCLEWIGYINPSSGYTNYNQKFTDRATLTADKSTSTAYMELSSLRSEDTAVYYCARSDGSSSKWYFDVWGQGTTVTVSSGGGGSGGGGSGGGGSEIVLTQSPDFQSATPKEKVTITCRASSSVSYIHWYQQKPGSSPKAWISATSNLASGVPSRFSGSGSGTSYTLTINRVEAEDAATYYCQQWSSNPFTFGCGTKLEIK

SEQ ID NO: 130 - protein, 242 aa, Artificial Sequence
  source: /note=“Description of Artificial Sequence Synthetic polypeptide”
QVQLVQSGAEVKKPGASVKMSCKASGYTFTFYTMHWLKQAPGQGLEWIGYINPSSGYTNYNQKFTDRATLTADKSTSTAYMELSSLRSEDTAVYYCARSDGSSSKWYFDVWGQGTTVTVSSGGGGSGGGGSGGGGSEIVLTQSPDFQSATPKEKVTITCRASSSVSYIHWYQQKPGSSPKAWISATSNLASGVPSRFSGSGSGTSYTLTINRVEAEDAATYYCQQWSSNPFTFGQGTKLEIK

SEQ ID NO: 131 - protein, 240 aa, Artificial Sequence
  source: /note=“Description of Artificial Sequence Synthetic polypeptide”
EVQLVQSGAEVKKPGATVKISCKASGFNIKDYYIHWVNQAPGKCLEWIGRIDPEDGDIAYAPKFQDRVTLTVDTSTDTAYLELSSLRSEDTAVYYCTTGNYYAMDFWGQGTTVTVSSGGGGSGGGGSGGGGSQIVLTQSPATLSASPGERVTLSCTASSSVSSSYLHWYQQKPGSSPKLWIYSTSNLASGVPARFSGSGPGTSYTLTISSMEPEDAATYYCHQYHRSPPTFGCGTKLEIK

SEQ ID NO: 132 - protein, 240 aa, Artificial Sequence
  source: /note=“Description of Artificial Sequence Synthetic polypeptide”
EVQLVQSGAEVKKPGATVKISCKASGFNIKDYYIHWVNQAPGKGLEWIGRIDPEDGDIAYAPKFQDRVTLTVDTSTDTAYLELSSLRSEDTAVYYCTTGNYYAMDFWGQGTTVTVSSGGGGSGGGGSGGGGSQIVLTQSPATLSASPGERVTLSCTASSSVSSSYLHWYQQKPGSSPKLWIYSTSNLASGVPARFSGSGPGTSYTLTISSMEPEDAATYYCHQYHRSPPTFGQGTKLEIK

SEQ ID NO: 133 - protein, 50 aa, Artificial Sequence
  source: /note=“Description of Artificial Sequence Synthetic polypeptide”
  SITE (1)..(50): /note=“This sequence may encompass 1-10′Gly Gly Gly Gly Ser′ repeating units”
  source: /note=“See specification as filed for detailed description of substitutions and preferred embodiments”
GGGGSGGGGSGGGGSGGGGSGGGGSGGGGSGGGGSGGGGSGGGGSGGGGS

(19) United States
(12) Patent Application Publication
(10) Pub. No.: US 2024/0084014 A1
(43) Pub. Date: Mar. 14, 2024

(54) MULTISPECIFIC BINDING COMPOUNDS THAT BIND TO PD-L1

(71) Applicant: QLSF Biotherapeutics, Inc., South San Francisco, CA (US)

(72) Inventors: Shenda Gu, South San Francisco, CA (US); Shihao Chen, San Mateo, CA (US); Lauren Schwimmer, South San Francisco, CA (US)

(21) Appl. No.: 18/249,221

(22) PCT Filed: Oct. 15, 2021
(86) PCT No.: PCT/US2021/055225

§ 371 Date: Apr. 14, 2023

Related U.S. Application Data
(60) Provisional application No. 63/093,109, filed on Oct. 16, 2020.

(51) Int. Cl. C07K 16/28; A61P 35/00; C07K 16/24

(52) U.S. Cl. CPC C07K 16/2827; A61P 35/00; C07K 16/244; C07K 16/2803; C07K 16/2878; C07K 2317/31; C07K 2317/33; C07K 2317/52; C07K 2317/55; C07K 2317/622; C07K 2317/76; C07K 2317/92

(57) ABSTRACT

Multispecific binding compounds that bind to PD-L1 are disclosed, along with methods of making such binding compounds, compositions, including pharmaceutical compositions, comprising such binding compounds, and their use to treat disorders that are characterized by the expression of PD-L1.

### CROSS-REFERENCE TO RELATED APPLICATIONS

This application claims priority benefit of the filing date of U.S. Provisional Patent Application Ser. No. 63/093,109, filed on Oct. 16, 2020, the disclosure of which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Dec. 30, 2021, is named QLS-0002-WO_SL.txt and is 228,930 bytes in size.

### FIELD OF THE INVENTION

The present invention concerns multispecific binding compounds that bind to PD-L1. The invention further concerns methods of making such binding compounds, compositions, including pharmaceutical compositions, comprising such binding compounds, and their use to treat disorders that are characterized by the expression of PD-L1.

### BACKGROUND OF THE INVENTION

Cancer is a leading cause of death worldwide. In advanced metastatic cancer, traditional therapeutic regimens, such as radiation therapy and chemotherapy, are marginally effective in prolonging survival. Targeted therapies, such as small molecule inhibitors and inhibitory monoclonal antibodies, have led to significant improvement in managing disease progression, but are nonetheless limited to subsets of cancer harboring specific mutations or those overexpressing targetable receptors. In addition, resistance to these therapies is common, as tumor cells can further mutate or switch to alternative signaling pathways, bypassing the inhibitory effect of the drugs. Immunotherapy holds new promises in fighting cancer by leveraging the body's own immune system. Checkpoint inhibitors targeting PD-1 and CTLA-4, for example, have led the way in advancing research and development in this field. The development of multispecific antibodies has allowed for new therapeutic modalities.

PD-L1 multispecific antibodies aim to target PD-L1 and one or more co-stimulatory receptors, such as 4-1BB. Such antibodies work in several ways. First, they bind to tumor cells expressing PD-L1, which are immunosuppressive. Second, they fulfill the role of a canonical checkpoint inhibitor by blocking the interaction of PD-L1 with its receptor PD-1. Third, they cross-link a co-stimulatory target, such as 4-1BB, on T cells, which in turn stimulates T cell proliferation only in the presence of tumor cells expressing PD-L1. In addition, the constant region of the antibody can contain mutations that eliminate Fc gamma receptor mediated functions, such as antibody dependent cellular cytotoxicity (ADCC) and complement dependent cytotoxicity (CDC). Accordingly, multispecific antibodies against PD-L1 have a strong immune response that is limited to tumor tissues, sparing normal tissues from unwanted toxicity that is often observed with standalone co-stimulatory antibodies.

### SUMMARY OF THE INVENTION

Aspects of the invention include bispecific antibodies that bind to PD-L1 and 4-1BB, comprising: two binding units that bind to PD-L1, each comprising: a heavy chain variable region comprising: a CDR1 sequence comprising SEQ ID NO: 1 or 4; a CDR2 sequence comprising SEQ ID NO: 2 or 5; and a CDR3 sequence comprising SEQ ID NO: 3 or 6; and a light chain variable region comprising: a CDR1 sequence comprising SEQ ID NO: 7 or 10; a CDR2 sequence comprising SEQ ID NO: 8 or 11; and a CDR3 sequence comprising SEQ ID NO: 9 or 12; and two binding units that bind to 4-1BB, each comprising a single chain Fv (scFv) comprising: a heavy chain variable region comprising: a CDR1 sequence comprising SEQ ID NO: 13 or 16; a CDR2 sequence comprising SEQ ID NO: 14 or 17; and a CDR3 sequence comprising SEQ ID NO: 15 or 18; and a light chain variable region comprising: a CDR1 sequence comprising SEQ ID NO: 19 or 22; a CDR2 sequence comprising SEQ ID NO: 20 or 23; and a CDR3 sequence comprising SEQ ID NO: 21 or 24.

In some embodiments, the two binding units that bind to PD-L1 each comprise: a heavy chain variable region comprising: a CDR1 sequence comprising SEQ ID NO: 1; a CDR2 sequence comprising SEQ ID NO: 2; and a CDR3 sequence comprising SEQ ID NO: 3; and a light chain variable region comprising: a CDR1 sequence comprising SEQ ID NO: 7; a CDR2 sequence comprising SEQ ID NO: 8; and a CDR3 sequence comprising SEQ ID NO: 9. In some embodiments, the two binding units that bind to PD-L1 each comprise: a heavy chain variable region comprising: a CDR1 sequence comprising SEQ ID NO: 4; a CDR2 sequence comprising SEQ ID NO: 5; and a CDR3 sequence comprising SEQ ID NO: 6; and a light chain variable region comprising: a CDR1 sequence comprising SEQ ID NO: 10; a CDR2 sequence comprising SEQ ID NO: 11; and a CDR3 sequence comprising SEQ ID NO: 12.

In some embodiments, the two binding units that bind to 4-1BB each comprise: a heavy chain variable region comprising: a CDR1 sequence comprising SEQ ID NO: 13; a CDR2 sequence comprising SEQ ID NO: 14; and a CDR3 sequence comprising SEQ ID NO: 15; and a light chain variable region comprising: a CDR1 sequence comprising SEQ ID NO: 19; a CDR2 sequence comprising SEQ ID NO: 20; and a CDR3 sequence comprising SEQ ID NO: 21.

In some embodiments, the two binding units that bind to 4-1BB each comprise: a heavy chain variable region comprising: a CDR1 sequence comprising SEQ ID NO: 16; a CDR2 sequence comprising SEQ ID NO: 17; and a CDR3 sequence comprising SEQ ID NO: 18; and a light chain variable region comprising: a CDR1 sequence comprising SEQ ID NO: 22; a CDR2 sequence comprising SEQ ID NO: 23; and a CDR3 sequence comprising SEQ ID NO: 24.

In some embodiments, the CDR1, CDR2 and CDR3 sequences in each binding unit are present in a human VH or a human VL framework. In some embodiments, the two binding units that bind to PD-L1 each comprise a heavy chain variable region comprising a sequence having at least 95% sequence identity to SEQ ID NO: 25. In some embodiments, the two binding units that bind to PD-L1 each comprise a heavy chain variable region comprising SEQ ID NO: 25. In some embodiments, the two binding units that bind to PD-L1 each comprise a light chain variable region comprising a sequence having at least 95% sequence identity to SEQ ID NO: 27. In some embodiments, the two binding units that bind to PD-L1 each comprise a light chain variable region comprising SEQ ID NO: 27. In some embodiments, the two binding units that bind to PD-L1 each comprise a heavy chain variable region comprising a sequence having at least 95% sequence identity to SEQ ID NO: 26. In some embodiments, the two binding units that bind to PD-L1 each comprise a heavy chain variable region comprising SEQ ID NO: 26. In some embodiments, the two binding units that bind to PD-L1 each comprise a light chain variable region comprising a sequence having at least 95% sequence identity to SEQ ID NO: 28. In some embodiments, the two binding units that bind to PD-L1 each comprise a light chain variable region comprising SEQ ID NO: 28.

In some embodiments, the two binding units that bind to 4-1BB each comprise a heavy chain variable region comprising a sequence having at least 95% sequence identity to SEQ ID NO: 29. In some embodiments, the two binding units that bind to 4-1BB each comprise a heavy chain variable region comprising SEQ ID NO: 29. In some embodiments, the two binding units that bind to 4-1BB each comprise a light chain variable region comprising a sequence having at least 95% sequence identity to SEQ ID NO: 31. In some embodiments, the two binding units that bind to 4-1BB each comprise a light chain variable region comprising SEQ ID NO: 31. In some embodiments, the two binding units that bind to 4-1BB each comprise a heavy chain variable region comprising a sequence having at least 95% sequence identity to SEQ ID NO: 30. In some embodiments, the two binding units that bind to 4-1BB each comprise a heavy chain variable region comprising SEQ ID NO: 30. In some embodiments, the two binding units that bind to 4-1BB each comprise a light chain variable region comprising a sequence having at least 95% sequence identity to SEQ ID NO: 32. In some embodiments, the two binding units that bind to 4-1BB each comprise a light chain variable region comprising SEQ ID NO: 32.

In some embodiments, the antibodies further comprise a heavy chain constant region sequence that comprises a CH1 domain, a hinge region sequence, a CH2 domain, and a CH3 domain. In some embodiments, the heavy chain constant region sequence comprises a wild type human IgG1 constant region sequence (SEQ ID NO: 92). In some embodiments, the heavy chain constant region sequence comprises an L234A mutation, an L235A mutation, a G237A mutation, or any combination thereof. In some embodiments, the heavy chain constant region sequence comprises SEQ ID NO: 93.

In some embodiments, the antibodies further comprise a light chain constant region sequence. In some embodiments, the light chain constant region sequence comprises a human kappa light chain constant region sequence (SEQ ID NO: 91). In some embodiments, the light chain constant region sequence comprises a human lambda light chain constant region sequence.

In some embodiments, in each of the binding units that bind to 4-1BB, the heavy chain variable region and the light chain variable region are connected by a linker sequence. In some embodiments, the linker sequence comprises a G₄S linker sequence (SEQ ID NO: 36). In some embodiments, the G₄S linker sequence (SEQ ID NO: 36) comprises SEQ ID NO: 36, SEQ ID NO: 37, or SEQ ID NO: 38.

In some embodiments, each of the second binding units is connected to a C-terminus of the heavy chain constant region sequence by a linker sequence. In some embodiments, the linker sequence comprises a G₄S linker sequence (SEQ ID NO: 36). In some embodiments, the G₄S linker sequence (SEQ ID NO: 36) comprises SEQ ID NO: 36, SEQ ID NO: 37, or SEQ ID NO: 38.

Aspects of the invention include a bispecific antibody that binds to PD-L1 and 4-1BB, comprising: (a) a first light chain polypeptide comprising the sequence of SEQ ID NO: 43; (b) a first heavy chain polypeptide comprising the sequence of SEQ ID NO: 41; (c) a second light chain polypeptide comprising the sequence of SEQ ID NO: 43; and (d) a second heavy chain polypeptide comprising the sequence of SEQ ID NO: 41.

Aspects of the invention include a bispecific antibody that binds to PD-L1 and 4-1BB, comprising: (a) a first light chain polypeptide comprising the sequence of SEQ ID NO: 44; (b) a first heavy chain polypeptide comprising the sequence of SEQ ID NO: 42; (c) a second light chain polypeptide comprising the sequence of SEQ ID NO: 44; and (d) a second heavy chain polypeptide comprising the sequence of SEQ ID NO: 42.

Aspects of the invention include bispecific antibodies that bind to PD-L1 and CD47, comprising: a first binding unit that binds to PD-L1, comprising: a heavy chain variable region comprising: a CDR1 sequence comprising SEQ ID NO: 1 or 4; a CDR2 sequence comprising SEQ ID NO: 2 or 5; and a CDR3 sequence comprising SEQ ID NO: 3 or 6; and a light chain variable region comprising: a CDR1 sequence comprising SEQ ID NO: 7 or 10; a CDR2 sequence comprising SEQ ID NO: 8 or 11; and a CDR3 sequence comprising SEQ ID NO: 9 or 12; and a second binding unit that binds to CD47, comprising a single chain Fv (scFv) comprising: a heavy chain variable region comprising: a CDR1 sequence comprising SEQ ID NO: 50 or 53; a CDR2 sequence comprising SEQ ID NO: 51 or 54; and a CDR3 sequence comprising SEQ ID NO: 52 or 55; and a light chain variable region comprising: a CDR1 sequence comprising SEQ ID NO: 56 or 59; a CDR2 sequence comprising SEQ ID NO: 57 or 60; and a CDR3 sequence comprising SEQ ID NO: 58 or 61.

In some embodiments, the first binding unit that binds to PD-L1 comprises: a heavy chain variable region comprising: a CDR1 sequence comprising SEQ ID NO: 1; a CDR2 sequence comprising SEQ ID NO: 2; and a CDR3 sequence comprising SEQ ID NO: 3; and a light chain variable region comprising: a CDR1 sequence comprising SEQ ID NO: 7; a CDR2 sequence comprising SEQ ID NO: 8; and a CDR3 sequence comprising SEQ ID NO: 9. In some embodiments, the first binding unit that binds to PD-L1 comprises: a heavy chain variable region comprising: a CDR1 sequence comprising SEQ ID NO: 4; a CDR2 sequence comprising SEQ ID NO: 5; and a CDR3 sequence comprising SEQ ID NO: 6; and a light chain variable region comprising: a CDR1 sequence comprising SEQ ID NO: 10; a CDR2 sequence comprising SEQ ID NO: 11; and a CDR3 sequence comprising SEQ ID NO: 12.

In some embodiments, the second binding unit that binds to CD47 comprises: a heavy chain variable region comprising: a CDR1 sequence comprising SEQ ID NO: 50; a CDR2 sequence comprising SEQ ID NO: 51; and a CDR3 sequence comprising SEQ ID NO: 52; and a light chain variable region comprising: a CDR1 sequence comprising SEQ ID NO: 56; a CDR2 sequence comprising SEQ ID NO: 57; and a CDR3 sequence comprising SEQ ID NO: 58. In some embodiments, the second binding unit that binds to CD47 comprises: a heavy chain variable region comprising: a CDR1 sequence comprising SEQ ID NO: 53; a CDR2 sequence comprising SEQ ID NO: 54; and a CDR3 sequence comprising SEQ ID NO: 55; and a light chain variable region comprising: a CDR1 sequence comprising SEQ ID NO: 59; a CDR2 sequence comprising SEQ ID NO: 60; and a CDR3 sequence comprising SEQ ID NO: 61.

In some embodiments, the CDR1, CDR2 and CDR3 sequences in each binding unit are present in a human VH or a human VL framework. In some embodiments, the first binding unit that binds to PD-L1 comprises a heavy chain variable region comprising a sequence having at least 95% sequence identity to SEQ ID NO: 25. In some embodiments, the first binding unit that binds to PD-L1 comprises a heavy chain variable region comprising SEQ ID NO: 25. In some embodiments, the first binding unit that binds to PD-L1 comprises a light chain variable region comprising a sequence having at least 95% sequence identity to SEQ ID NO: 27. In some embodiments, the first binding unit that binds to PD-L1 comprises a light chain variable region comprising SEQ ID NO: 27. In some embodiments, the first binding unit that binds to PD-L1 comprises a heavy chain variable region comprising a sequence having at least 95% sequence identity to SEQ ID NO: 26. In some embodiments, the first binding unit that binds to PD-L1 comprises a heavy chain variable region comprising SEQ ID NO: 26. In some embodiments, the first binding unit thats bind to PD-L1 comprises a light chain variable region comprising a sequence having at least 95% sequence identity to SEQ ID NO: 28. In some embodiments, the first binding unit that binds to PD-L1 comprises a light chain variable region comprising SEQ ID NO: 28.

In some embodiments, the second binding unit that binds to CD47 comprises a heavy chain variable region comprising a sequence having at least 95% sequence identity to SEQ ID NO: 62. In some embodiments, the second binding unit that binds to CD47 comprises a heavy chain variable region comprising SEQ ID NO: 62. In some embodiments, the second binding unit that binds to CD47 comprises a light chain variable region comprising a sequence having at least 95% sequence identity to SEQ ID NO: 64. In some embodiments, the second binding unit that binds to CD47 comprises a light chain variable region comprising SEQ ID NO: 64. In some embodiments, the second binding unit that binds to CD47 comprises a heavy chain variable region comprising a sequence having at least 95% sequence identity to SEQ ID NO: 63. In some embodiments, the second binding unit that binds to CD47 comprises a heavy chain variable region comprising SEQ ID NO: 63. In some embodiments, the second binding unit that binds to CD47 comprises a light chain variable region comprising a sequence having at least 95% sequence identity to SEQ ID NO: 65. In some embodiments, the second binding unit that binds to CD47 comprises a light chain variable region comprising SEQ ID NO: 65.

In some embodiments, the bispecific antibody further comprises a heavy chain constant region sequence that comprises a CH1 domain, a hinge region sequence, a CH2 domain, and a CH3 domain. In some embodiments, the heavy chain constant region sequence comprises a wild type human IgG1 constant region sequence (SEQ ID NO: 92). In some embodiments, the heavy chain constant region sequence comprises an L234A mutation, an L235A mutation, a G237A mutation, or any combination thereof. In some embodiments, the heavy chain constant region sequence comprises SEQ ID NO: 93.

In some embodiments, the bispecific antibody further comprises a light chain constant region sequence. In some embodiments, the light chain constant region sequence comprises a human kappa light chain constant region sequence (SEQ ID NO: 91). In some embodiments, the light chain constant region sequence comprises a human lambda light chain constant region sequence.

In some embodiments, in the second binding unit that binds to CD47, the heavy chain variable region and the light chain variable region are connected by a linker sequence. In some embodiments, the linker sequence comprises a G₄S linker sequence (SEQ ID NO: 36). In some embodiments, the G₄S linker sequence (SEQ ID NO: 36) comprises SEQ ID NO: 36, SEQ ID NO: 37, or SEQ ID NO: 38.

In some embodiments, each of the second binding units is connected to a C-terminus of the heavy chain constant region sequence by a linker sequence. In some embodiments, the linker sequence comprises a G₄S linker sequence (SEQ ID NO: 36). In some embodiments, the G₄S linker sequence (SEQ ID NO: 36) comprises SEQ ID NO: 36, SEQ ID NO: 37, or SEQ ID NO: 38. In some embodiments, the bispecific antibody further comprises a heavy chain constant region comprising one or more knobs-in-holes mutations that facilitate heterodimerization of two different heavy chain polypeptides.

Aspects of the invention include a bispecific antibody that binds to PD-L1 and CD47, comprising: (a) a first light chain polypeptide comprising the sequence of SEQ ID NO: 66; (b) a first heavy chain polypeptide comprising the sequence of SEQ ID NO: 67; and (c) a second heavy chain polypeptide comprising the sequence of SEQ ID NO: 68.

Aspects of the invention include a bispecific antibody that binds to PD-L1 and CD47, comprising: (a) a first light chain polypeptide comprising the sequence of SEQ ID NO: 69; (b) a first heavy chain polypeptide comprising the sequence of SEQ ID NO: 70; and (c) a second heavy chain polypeptide comprising the sequence of SEQ ID NO: 71.

Aspects of the invention include antibodies that bind to PD-L1 and comprises one or more IL15 polypeptides fused to a C-terminus of heavy chain polypeptide subunit of the bispecific antibody, comprising: a first binding unit that binds to PD-L1, comprising: a heavy chain variable region comprising: a CDR1 sequence comprising SEQ ID NO: 1 or 4; a CDR2 sequence comprising SEQ ID NO: 2 or 5; and a CDR3 sequence comprising SEQ ID NO: 3 or 6; and a light chain variable region comprising: a CDR1 sequence comprising SEQ ID NO: 7 or 10; a CDR2 sequence comprising SEQ ID NO: 8 or 11; and a CDR3 sequence comprising SEQ ID NO: 9 or 12; and an IL15 polypeptide comprising a sequence having at least 95% identity to any one of SEQ ID NOs: 86-90.

In some embodiments, the first binding unit that binds to PD-L1 comprises: a heavy chain variable region comprising: a CDR1 sequence comprising SEQ ID NO: 1; a CDR2 sequence comprising SEQ ID NO: 2; and a CDR3 sequence comprising SEQ ID NO: 3; and a light chain variable region comprising: a CDR1 sequence comprising SEQ ID NO: 7; a CDR2 sequence comprising SEQ ID NO: 8; and a CDR3 sequence comprising SEQ ID NO: 9. In some embodiments, the first binding unit that binds to PD-L1 comprises: a heavy chain variable region comprising: a CDR1 sequence comprising SEQ ID NO: 4; a CDR2 sequence comprising SEQ ID NO: 5; and a CDR3 sequence comprising SEQ ID NO: 6; and a light chain variable region comprising: a CDR1 sequence comprising SEQ ID NO: 10; a CDR2 sequence comprising SEQ ID NO: 11; and a CDR3 sequence comprising SEQ ID NO: 12.

In some embodiments, the IL15 polypeptide comprises a sequence of any one of SEQ ID NOs: 86-90. In some embodiments, the IL15 polypeptide is connected to the heavy chain polypeptide subunit of the antibody by a linker sequence. In some embodiments, the linker sequence comprises a sequence of any one of SEQ ID NOs: 36, 37, 38, 49, 120, 121, 122, 123, 124, 125, 126, 127 or 128.

In some embodiments, the CDR1, CDR2 and CDR3 sequences are present in a human VH or a human VL framework. In some embodiments, the first binding unit that binds to PD-L1 comprises a heavy chain variable region comprising a sequence having at least 95% sequence identity to SEQ ID NO: 25. In some embodiments, the first binding unit that binds to PD-L1 comprises a heavy chain variable region comprising SEQ ID NO: 25. In some embodiments, the first binding unit that binds to PD-L1 comprises a light chain variable region comprising a sequence having at least 95% sequence identity to SEQ ID NO: 27. In some embodiments, the first binding unit that binds to PD-L1 comprises a light chain variable region comprising SEQ ID NO: 27. In some embodiments, the first binding unit that binds to PD-L1 comprises a heavy chain variable region comprising a sequence having at least 95% sequence identity to SEQ ID NO: 26. In some embodiments, the first binding unit that binds to PD-L1 comprises a heavy chain variable region comprising SEQ ID NO: 26. In some embodiments, the first binding unit that bind to PD-L1 comprises a light chain variable region comprising a sequence having at least 95% sequence identity to SEQ ID NO: 28. In some embodiments, the first binding unit that binds to PD-L1 comprises a light chain variable region comprising SEQ ID NO: 28.

In some embodiments, the antibody further comprises a heavy chain constant region sequence that comprises a CH1 domain, a hinge region sequence, a CH2 domain, and a CH3 domain. In some embodiments, the heavy chain constant region sequence comprises a wild type human IgG1 constant region sequence (SEQ ID NO: 92). In some embodiments, the heavy chain constant region sequence comprises an L234A mutation, an L235A mutation, a G237A mutation, or any combination thereof. In some embodiments, the heavy chain constant region sequence comprises SEQ ID NO: 93.

In some embodiments, the antibody further comprises a light chain constant region sequence. In some embodiments, the light chain constant region sequence comprises a human kappa light chain constant region sequence (SEQ ID NO: 91). In some embodiments, the light chain constant region sequence comprises a human lambda light chain constant region sequence.

In some embodiments, the antibody further comprises a heavy chain constant region comprising one or more knobs-in-holes mutations that facilitate heterodimerization of two different heavy chain polypeptides.

Aspects of the invention include a bispecific antibody that binds to PD-L1 and comprises an IL15 polypeptide fused to a C-terminus of each heavy chain polypeptide subunit, comprising: (a) a first light chain polypeptide comprising the sequence of SEQ ID NO: 104; (b) a first heavy chain polypeptide comprising the sequence of SEQ ID NO: 105; (c) a second heavy chain polypeptide comprising the sequence of SEQ ID NO: 105; and (d) a second light chain polypeptide comprising the sequence of SEQ ID NO: 104.

Aspects of the invention include a bispecific antibody that binds to PD-L1 and comprises an IL15 polypeptide fused to a C-terminus of each heavy chain polypeptide subunit, comprising: (a) a first light chain polypeptide comprising the sequence of SEQ ID NO: 106; (b) a first heavy chain polypeptide comprising the sequence of SEQ ID NO: 107; (c) a second heavy chain polypeptide comprising the sequence of SEQ ID NO: 107; and (d) a second light chain polypeptide comprising the sequence of SEQ ID NO: 106.

Aspects of the invention include a bispecific antibody that binds to PD-L1 and comprises an IL15 polypeptide fused to a C-terminus of each heavy chain polypeptide subunit, comprising: (a) a first light chain polypeptide comprising the sequence of SEQ ID NO: 108; (b) a first heavy chain polypeptide comprising the sequence of SEQ ID NO: 109; (c) a second heavy chain polypeptide comprising the sequence of SEQ ID NO: 110; and (d) a second light chain polypeptide comprising the sequence of SEQ ID NO: 108.

Aspects of the invention include a bispecific antibody that binds to PD-L1 and comprises an IL15 polypeptide fused to a C-terminus of one heavy chain polypeptide subunit, comprising: (a) a first light chain polypeptide comprising the sequence of SEQ ID NO: 111; (b) a first heavy chain polypeptide comprising the sequence of SEQ ID NO: 112; (c) a second heavy chain polypeptide comprising the sequence of SEQ ID NO: 113; and (d) a second light chain polypeptide comprising the sequence of SEQ ID NO: 111.

Aspects of the invention include a bispecific antibody that binds to PD-L1 and comprises an IL15 polypeptide fused to a C-terminus of one heavy chain polypeptide subunit, comprising: (a) a first light chain polypeptide comprising the sequence of SEQ ID NO: 114; (b) a first heavy chain polypeptide comprising the sequence of SEQ ID NO: 115; (c) a second heavy chain polypeptide comprising the sequence of SEQ ID NO: 116; and (d) a second light chain polypeptide comprising the sequence of SEQ ID NO: 114.

Aspects of the invention include a bispecific antibody that binds to PD-L1 and comprises an IL15 polypeptide fused to a C-terminus of each heavy chain polypeptide subunit, comprising: (a) a first light chain polypeptide comprising the sequence of SEQ ID NO: 117; (b) a first heavy chain polypeptide comprising the sequence of SEQ ID NO: 118; (c) a second heavy chain polypeptide comprising the sequence of SEQ ID NO: 119; and (d) a second light chain polypeptide comprising the sequence of SEQ ID NO: 117.

Aspects of the invention include pharmaceutical compositions comprising an antibody as described herein.

Aspects of the invention include methods for the treatment of a disorder characterized by expression of PD-L1, comprising administering to a subject with said disorder an antibody as described herein, or a pharmaceutical composition as described herein.

Aspects of the invention include use of an antibody as described herein in the preparation of a medicament for the treatment of a disorder characterized by expression of PD-L1.

Aspects of the invention include an antibody as described herein for use in the treatment of a disorder characterized by expression of PD-L1. In some embodiments, the disorder is cancer.

Aspects of the invention include a polynucleotide encoding an antibody as described herein, a vector comprising a polynucleotide as described herein, and a cell comprising a vector of claim as described herein.

Aspects of the invention include a method of producing an antibody as described herein, comprising growing a cell as described herein under conditions permissive for expression of the antibody, and isolating the antibody from the cell.

Aspects of the invention include a method of treatment, comprising administering to an individual in need an effective dose of an antibody as described herein, or a pharmaceutical composition as described herein.

These and further aspects will be further explained in the rest of the disclosure, including the Examples.

### BRIEF DESCRIPTION OF THE DRAWINGS

FIG. 1, panel A, is a graph showing binding of the indicated antibody constructs to HEK293 cells expressing human PD-L1.

FIG. 1, panel B, is a graph showing binding of the indicated antibody constructs to HEK293 cells expressing human 4-1BB.

FIG. 1, panel C, is a table showing EC50 values for human PD-L1 and human 4-1BB for the indicated antibody constructs.

FIG. 2, panels A and B, are graphs showing binding kinetics of antibody constructs to HIS tagged PD-L1 and 4-1BB, respectively.

FIG. 2, panel C, is a table showing KD values for binding to PD-L1 and 4-1BB.

FIG. 3, panel A, is a graph showing binding of the indicated antibody constructs to HEK293 cells expressing cyno PD-L1.

FIG. 3, panel B, is a graph showing binding of the indicated antibody constructs to HEK293 cells expressing cyno 4-1BB.

FIG. 3, panel C, is a table showing EC50 values for cyno PD-L1 and cyno 4-1BB for the indicated antibody constructs.

FIG. 4, panel A, is a graph showing PD-1 blocking activity of the indicated antibody constructs in HEK293 cells expressing PD-L1.

FIG. 4, panel B, is a graph showing 4-1BBL blocking activity of the indicated antibody constructs in HEK293 cells expressing 4-1BB.

FIG. 4, panel C, is a table showing IC50 values for PD-1 and 4-1BBL for the indicated antibody constructs.

FIG. 5, panel A, is a graph showing bifunctional ELISA binding of the indicated antibody constructs as a function of concentration.

FIG. 5, panel B, is a graph showing NF-kB reporter activity for the indicated antibody constructs as function of antibody concentration.

FIG. 6, panels A and B, are graphs showing IL2 release and IFN⋅ release, respectively, from human PBMCs stimulated with anti-CD3 antibody (OKT3) and then co-cultured with PD-L1+ A431 cells, together with the indicated antibody constructs at the indicated concentrations.

FIG. 6, panel C, is a graph showing IL2 release from human PBMCs stimulated with anti-CD3 antibody (OKT3) and then cultured with and without PD-L1+ A431 cells, together with the indicated antibody constructs at the indicated concentrations.

FIG. 7, panel A, is a graph showing IL2 release in an SEB stimulation assay using the indicated antibody constructs at the indicated concentrations.

FIG. 7, panel B, is a graph showing CD8+ T-cell proliferation in the presence of anti-CD3 antibody (OKT3) and PD-L1+ A431 cells with the indicated antibody constructs at the indicated concentrations.

FIG. 8, panel A, is a graph showing tumor volume as a function of days post initial dose for an MC38 mouse tumor model using treatment with the indicated antibody constructs at the indicated doses.

FIG. 8, panel B, is a graph showing tumor infiltrating immune cells (CD8+ T-cells) for each dose group taken at the end of the study.

FIG. 9, panel A, is a graph showing tumor volume as a function of days post initial dose for an A431 human tumor model using treatment with the indicated antibody constructs at the indicated doses.

FIG. 9, panel B, is a graph showing tumor infiltrating immune cells (CD8+ T-cells) for each dose group taken at the end of the study.

FIG. 10 is a table showing calculated percentages of monomer, aggregate and fragment in an HPLC-SEC profile of QL301 taken from an accelerated temperature stress test.

FIG. 11, panel A, is a graph showing bifunctional ELISA binding as a function of antibody concentration for QL301 after incubation in human serum for 7 days, and as a stock control.

FIG. 11, panel B, is a graph showing IL2 release from PBMCs in an SEB stimulation assay as a function of antibody concentration using the human serum-incubated and stock control QL301 antibody at the indicated concentrations.

FIG. 12, panel A, is a schematic illustration of a PD-L1-CD47 bispecific antibody.

FIG. 12, panel B, is a graph showing ELISA binding to PD-L1 and CD47 as a function of antibody concentration.

FIG. 13, panels A-E, are a series of graphs showing binding of the indicated antibody constructs to the indicated cells as a function of antibody concentration.

FIG. 14 is a graph showing PD-1 blocking activity on stimulated A431 cells by the indicated CD47-PD-L1 bispecific antibody constructs, as a function of antibody concentration.

FIG. 15 is a graph showing PD-1 blocking activity on huPD-L1+ HEK293 cells by the indicated CD47-PD-L1 bispecific antibody constructs, as a function of antibody concentration.

FIG. 16, panel A, is a graph showing SIRP⋅ blocking on A431 cells by the indicated CD47-PD-L1 bispecific antibody constructs, as a function of antibody concentration.

FIG. 16, panel B, is a graph showing SIRP⋅ blocking on huCD47+ CHO cells by the indicated CD47-PD-L1 bispecific antibody constructs, as a function of antibody concentration.

FIG. 17, panel A, is a graph showing antibody-mediated phagocytosis of Raji cells using the indicated CD47-PD-L1 antibody constructs, at the indicated concentrations.

FIG. 17, panel B, is a graph showing antibody-mediated phagocytosis of MM.1S cells using the indicated CD47-PD-L1 antibody constructs, at the indicated concentrations.

FIG. 18, panels A and B, are graphs showing binding of the indicated CD47-PD-L1 bispecific antibodies to red blood cells, at the indicated antibody concentrations, for two different RBC donors.

FIG. 19 is an image showing hemagglutination of red blood cells induced by the indicated antibody constructs at the indicated concentrations.

FIG. 20, panels A-F, are a series of graphs showing tumor volume as a function of days in an A431, hPBMC co-graft tumor model in ICR-SCID mice. Dosing groups G1-G5 represent different antibody constructs, or a control (PBS).

FIG. 21, panels A-F, are graphs showing efficacy endpoints from the tumor model described in FIG. 20.

FIG. 22, panels A-F, are a series of graphs showing tumor volume as a function of days in an A431, hPBMC co-graft tumor model in NOD-SCID mice. Dosing groups G1-G5 represent different antibody constructs, or a control (PBS).

FIG. 23, panels A-F, are schematic illustrations of various bispecific antibody constructs comprising IL15 fusions at their C-termini.

FIG. 24, panels A-C, are a series of graphs showing binding of the indicated PD-L1-IL15 bispecific antibody constructs to the indicated cells, at the indicated antibody concentrations.

FIG. 25, panels A-C, are a series of graphs showing representative examples of proliferation of NK92 or M07e cells in response to the indicated PD-L1-IL15 bispecific antibody constructs, at the indicated concentrations.

FIG. 26, panels A and B, are graphs showing induction of pSTAT5 on MO7e cells using the indicated PD-L1-IL15 bispecific antibodies, or a monoclonal anti-PD-L1 or isotype control IL15 antibody, at the indicated concentrations.

FIG. 27, panels A-C, are a series of graphs showing proliferation of the indicated cell type in response to PD-L1-IL15 bispecific antibody exposure, at the indicated concentrations.

FIG. 28, panels A-D, are a series of graphs showing proliferation of the indicated cell type in response to PD-L1-IL15 bispecific antibody exposure, at the indicated concentrations.

FIG. 29, panels A-D, are a series of graphs showing proliferation of the indicated cell type in response to PD-L1-IL15 bispecific antibody exposure, at the indicated concentrations.

FIG. 29, panel E, is a table showing the antibodies used for staining (BioLegend catalog number listed).

FIG. 30, panels A-E, are a series of graphs showing cell count as a function of time for the indicated cell types, and the indicated PD-L1-IL15 bispecific antibody construct and dosing schedule.

FIG. 31, panels A-F, are a series of graphs showing cell count as a function of time for the indicated cell types, and the indicated PD-L1-IL15 bispecific antibody construct and dosing schedule.

FIG. 32, panels A-D, are a series of graphs showing the pharmacokinetic properties of the of the indicated PD-L1-IL15 bispecific antibody constructs in C57BL/6 and NSG mice.

FIG. 33, panels A-F, are a series of graphs showing tumor growth inhibition of MC38 murine colon cancer cells expressing PD-L1 with the indicated PD-L1-IL15 antibody construct, at the indicated doses.

FIG. 33, panel G, is a graph showing tumor volume as a function of days following tumor rechallenge.

FIG. 34, panels A-G, are a series of graphs showing tumor growth inhibition of an A431 xenograft co-grafted with human PBMC tumor model for the indicated PD-L1-IL15 bispecific antibody constructs, at the indicated doses.

FIG. 35, panels A-G, are a series of graphs showing phenotype analysis of cells taken from tumors isolated from an A431 xenograft co-grafted with human PBMC tumor model treated with the indicated PD-L1-IL15 bispecific antibody constructs, at the doses indicated in FIG. 34.

FIG. 36, panels A-G, are a series of graphs showing phenotype analysis of cells taken from tumors isolated from an A431 xenograft co-grafted with human PBMCs tumor model treated with the indicated PD-L1-IL15 bispecific antibody, at the doses indicated in FIG. 34.

FIG. 37, panels A-E, are a series of graphs showing tumor growth inhibition of MC38 murine colon cancer cells expressing PD-L1 with the indicated PD-L1-IL15 antibody construct, at the indicated doses, in C57BL/6 mice.

FIG. 38, panels A-F, are a series of graphs showing phenotype analysis of cells taken from tumors isolated from an MC38 murine colon cancer tumor model, treated with the indicated PD-L1-IL15 bispecific antibody constructs, at the doses indicated in FIG. 37.

FIG. 39, panels A-G, are a series of graphs showing tumor growth inhibition of NCI-H1650 cells co-grafted with human PBMCs in CD17-SCID mice for the indicated PD-L1-IL15 bispecific antibodies at the indicated doses.

FIG. 40, panel A, is a model of QL301, a bispecific PD-L1×4-1BB antibody with two identical binding regions that bind to PD-L1, and two identical scFvs that bind to 4-1BB.

FIG. 40, panel B, is an illustration of a tumor cell and a T-cell that have been cross-linked by a QL301 bispecific antibody.

FIG. 41, panels A and B, are graphs showing % phagocytosis of RBCs mediated by bispecific PD-L1×CD47 antibodies for two different donors.

FIG. 42 is a graph showing binding activity of the indicated PD-L1-IL15 antibody constructs.

FIG. 43 is a graph showing proliferation of NK92 cells in response to the indicated PD-L1-IL15 antibody constructs.

FIG. 44, panels A and B, are graphs showing AST and ALT levels observed in a repeated dose toxicology study in rhesus monkeys, using a PD-L1-4-1BB bispecific antibody construct in accordance with some embodiments of the invention.

FIG. 45 is a graph showing A375 tumor growth inhibition in a tumor model in NOG mice, using a PD-L1-CD47 bispecific antibody construct in accordance with some embodiments of the invention.

FIG. 46 is a graph showing Raji tumor growth inhibition in a tumor model in NOG mice, using a PD-L1-CD47 bispecific antibody construct in accordance with embodiments of the invention.

FIG. 47 is a graph showing red blood cell count observed in a repeated dose toxicology study in cynomolgus monkeys, using a PD-L1-CD47 bispecific antibody construct in accordance with embodiments of the invention.

FIG. 48 is a graph showing stimulation of cDC1 observed in an MC38 tumor model conducted in C57BL/6 mice, using a mouse cross-reactive surrogate of a PD-L1-IL15-T2A construct in accordance with embodiments of the invention.

### DETAILED DESCRIPTION OF THE PREFERRED EMBODIMENTS

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless indicated otherwise, antibody residues herein are numbered according to the Kabat numbering system (e.g., Kabat et al., Sequences of Immunological Interest. 5th Ed. Public Health Service, National Institutes of Health, Bethesda, Md. (1991)).

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

All references cited throughout the disclosure, including patent applications and publications, are incorporated by reference herein in their entirety.

### I. Definitions

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies mean residue numbering by the EU numbering system.

Antibodies, also referred to as immunoglobulins, conventionally comprise at least one heavy chain and one light chain, where the amino terminal domain of the heavy and light chains is variable in sequence, hence is commonly referred to as a variable region domain, or a variable heavy (VH) or variable light (VH) domain. The two domains conventionally associate to form a specific binding region, although as will be discussed here, specific binding can also be obtained with heavy chain-only variable sequences, and a variety of non-natural configurations of antibodies are known and used in the art.

A “functional” or “biologically active” antibody or binding compound is one capable of exerting one or more activities in structural, regulatory, biochemical or biophysical events. For example, a functional antibody or other binding compound may have the ability to specifically bind an antigen and the binding may in turn elicit or alter a cellular or molecular event such as signal transduction or enzymatic activity. A functional antibody or other binding compound may also block ligand activation of a receptor or act as an agonist or antagonist. The capability of an antibody or other binding compound to exert one or more activities depends on several factors, including proper folding and assembly of the polypeptide chains.

The term “antibody” herein is used in the broadest sense and specifically covers monoclonal antibodies, polyclonal antibodies, monomers, dimers, multimers, multispecific antibodies (e.g., bispecific antibodies), three chain antibodies, single chain Fv (scFv), nanobodies, etc., and also includes antibody fragments, so long as they exhibit the desired biological activity (Miller et al (2003) Jour. of Immunology 170:4854-4861). Antibodies may be murine, human, humanized, chimeric, or derived from other species.

The term antibody may reference a full-length heavy chain, a full length light chain, an intact immunoglobulin molecule; or an immunologically active portion of any of these polypeptides, i.e., a polypeptide that comprises an antigen binding site that immunospecifically binds an antigen of a target of interest or part thereof, such targets including but not limited to, a cancer cell, or cells that produce autoimmune antibodies associated with an autoimmune disease. The immunoglobulins disclosed herein can be of any type (e.g., IgG, IgE, IgM, IgD, and IgA), class (e.g., IgG1, IgG2, IgG3, IgG4, IgA1 and IgA2) or subclass of immunoglobulin molecule, including engineered subclasses with altered Fc portions that provide for reduced or enhanced effector cell activity. The immunoglobulins can be derived from any species.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. Monoclonal antibodies in accordance with the present invention can be made by the hybridoma method first described by Kohler et al. (1975) Nature 256:495, and can also be made via recombinant protein production methods (see, e.g., U.S. Pat. No. 4,816,567), for example.

The term “variable”, as used in connection with antibodies, refers to the fact that certain portions of the antibody variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a ⋅-sheet configuration, connected by three hypervariable regions, which form loops connecting, and in some cases forming part of, the ⋅-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, MD. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” residues 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

Exemplary CDR designations are shown herein, however one of skill in the art will understand that a number of definitions of the CDRs are commonly in use, including the Kabat definition (see “Zhao et al. A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlan et al. “Identification of specificity-determining residues in antibodies.” Faseb J. 1995; 9:133-139., each of which is herein specifically incorporated by reference.

The term “multispecific binding compound” as used herein means a binding compound that comprises two or more antigen binding sites. Multispecific binding compounds in accordance with embodiments of the invention can be antibody-like molecules comprising, consisting essentially of, or consisting of two, three, or four polypeptide subunits, any of which may comprise one or more variable region domains having binding affinity for a target antigen (e.g., PD-L1). In some embodiments, a multispecific binding compound comprises pair of variable region domains (e.g., a heavy chain variable region domain and a light chain variable region domain) that together form a binding unit. In some embodiments, a multispecific binding compound comprises a pair of variable region domains in a single chain Fv (scFv) format, wherein a first variable region domain and a second variable region domain are connected by a linker, and together form a binding unit. The subject multispecific binding compounds can have any suitable combination or configuration of binding units, including but not limited to the specific configurations described herein.

Multispecific binding compounds as described herein may belong to any immunoglobulin subclass, including IgG, IgM, IgA, IgD and IgE subclasses. In a particular embodiment, the multispecific binding compound is of the IgG1, IgG2, IgG3, or IgG4 subtype, in particular the IgG1 subtype. Modifications of CH domains that alter effector function are further described herein.

An “intact antibody chain” as used herein is one comprising a full length variable region and a full length constant region (Fc). An intact “conventional” antibody comprises an intact light chain and an intact heavy chain, as well as a light chain constant domain (CL) and heavy chain constant domains, CH1, hinge, CH2 and CH3 for secreted IgG. Other isotypes, such as IgM or IgA may have different CH domains. The constant domains may be native sequence constant domains (e.g., human native sequence constant domains) or amino acid sequence variants thereof. The intact antibody may have one or more “effector functions” which refer to those biological activities attributable to the Fc constant region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors. Constant region variants include those that alter the effector profile, binding to Fc receptors, and the like.

Depending on the amino acid sequence of the Fc (constant domain) of their heavy chains, antibodies and various antigen-binding proteins can be provided as different classes. There are five major classes of heavy chain Fc regions: IgA, IgD, IgE, IgG, and IgM, and several of these may be further divided into “subclasses” (isotypes), e.g., IgG1, IgG2, IgG3, IgG4, IgA, and IgA2. The Fc constant domains that correspond to the different classes of antibodies may be referenced as ⋅, ⋅, ⋅, ⋅, and ⋅, respectively. The subunit structures and three-dimensional configurations of different classes of immunoglobulins are well known. Ig forms include hinge-modifications or hingeless forms (Roux et al (1998) J. Immunol. 161:4083-4090; Lund et al (2000) Eur. J. Biochem. 267:7246-7256; US 2005/0048572; US 2004/0229310). The light chains of antibodies from any vertebrate species can be assigned to one of two types, called ⋅ and ⋅, based on the amino acid sequences of their constant domains.

A “functional Fc region” possesses an “effector function” of a native-sequence Fc region. Non-limiting examples of effector functions include C1q binding; CDC; Fc-receptor binding; ADCC; ADCP; down-regulation of cell-surface receptors (e.g., B-cell receptor), etc. Such effector functions generally require the Fc region to interact with a receptor, e.g., the FcγRI; FcγRIIA; Fc⋅RIIB1; FcγRIIB2; FcγRIIIA; FcγRIIIB receptors, and the low affinity FcRn receptor; and can be assessed using various assays known in the art. A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, preferably one or more amino acid substitution(s). Preferably, the variant Fc region has at least one amino acid substitution compared to a native-sequence Fc region or to the Fc region of a parent polypeptide, e.g., from about one to about ten amino acid substitutions, and preferably from about one to about five amino acid substitutions in a native-sequence Fc region or in the Fc region of the parent polypeptide. The variant Fc region herein will preferably possess at least about 80% homology with a native-sequence Fc region and/or with an Fc region of a parent polypeptide, and most preferably at least about 90% homology therewith, more preferably at least about 95% homology therewith.

The human IgG1 amino acid sequence is provided by UniProtKB No. P01857, which is incorporated by reference herein in its entirety. The human IgG2 amino acid sequence is provided by UniProtKB No. P01859, which is incorporated by reference herein in its entirety. The human IgG3 amino acid sequence is provided by UniProtKB No. P01860, which is incorporated by reference herein in its entirety. The human IgG4 amino acid sequence is provided by UniProtKB No. P01861, which is incorporated by reference herein in its entirety.

Variant Fc sequences may include three amino acid substitutions in the CH2 region to reduce FcγRI binding at EU index positions 234, 235, and 237 (see Duncan et al., (1988) Nature 332:563; Hezareh et al., (2001) J. Virology 75:12161; U.S. Pat. No. 5,624,821, the disclosures of which are incorporated herein by reference in their entireties). In some embodiments, a variant Fc sequence can include the following amino acid substitutions: L234A; L235A; and G237A. When these three amino acid substitutions are present in an IgG1 Fc sequence, they can be referred to as G1AAA.

Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 or IgG2 residues at positions 233-236 and IgG4 residues at positions 327, 330 and 331 greatly reduces ADCC and CDC (see, for example, Armour K L. et al., 1999 Eur J Immunol. 29(8):2613-24; and Shields R L. et al., 2001. J Biol Chem. 276(9):6591-604).

Other Fc variants are possible, including, without limitation, one in which a region capable of forming a disulfide bond is deleted, or in which certain amino acid residues are eliminated at the N-terminal end of a native Fc, or a methionine residue is added thereto. Thus, in some embodiments, one or more Fc portions of a binding compound can comprise one or more mutations in the hinge region to eliminate disulfide bonding. In yet another embodiment, the hinge region of an Fc can be removed entirely. In still another embodiment, a binding compound can comprise an Fc variant.

Further, an Fc variant can be constructed to remove or substantially reduce effector functions by substituting (mutating), deleting or adding amino acid residues to effect complement binding or Fc receptor binding. For example, and not limitation, a deletion may occur in a complement-binding site, such as a C1q-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

The term “Fc-region-comprising antibody” refers to an antibody that comprises an Fc region. The C-terminal lysine (residue 447 according to the EU numbering system) of the Fc region may be removed, for example, during purification of the antibody or by recombinant engineering of the nucleic acid encoding the antibody. Accordingly, an antibody having an Fc region according to this invention can comprise an antibody with or without K447.

Aspects of the invention include binding compounds having multi-specific configurations, which include, without limitation, bispecific, trispecific, etc. A large variety of methods and protein configurations are known and used in bispecific monoclonal antibodies (BsMAB), tri-specific antibodies, etc.

Various methods for the production of multivalent artificial antibodies have been developed by recombinantly fusing variable domains of two or more antibodies. In some embodiments, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to about 10, such as 2, 3, 4, 5, 6, 7, 8, or 9 (SEQ ID NO: 133). Non-limiting examples of such linkers include GGGGS (SEQ ID NO: 36) (n=1) and GGGGSGGGGS (SEQ ID NO: 37) (n=2). Other suitable linkers can also be used, and are described, for example, in Chen et al., Adv Drug Deliv Rev. 2013 Oct. 15; 65(10): 1357-69, the disclosure of which is incorporated herein by reference in its entirety. Additional linker sequences are described elsewhere herein, and can be incorporated into the subject antibodies in any suitable configuration.

Antibodies and multispecific binding compounds as described herein can be in the form of a dimer, in which two heavy chains are disulfide bonded or otherwise covalently or non-covalently attached to each other, and can optionally include an asymmetric interface between two or more of the CH domains to facilitate proper pairing between polypeptide chains (commonly referred to as a “knobs-into-holes” interface). Knobs into holes antibody engineering techniques for heavy chain heterodimerization are discussed, for example, in Ridgway et al., Protein Eng. 1996 July; 9(7):17-21, and U.S. Pat. No. 8,216,805, the disclosures of which are incorporated by reference herein in their entireties. An Fc region comprising an asymmetric interface can be referred to herein with the abbreviation “KiH”, meaning knobs-into-holes. For example, aspects of the invention include a variant Fc region sequence, such as a G1AAA sequence, that contains an asymmetric interface, and which is referred to herein as “G1AAA KiH”.

The terms “PD-L1” and “Programmed death ligand 1” refer to a PD-L1 protein of any human and non-human animal species, and specifically includes human PD-L1 as well as PD-L1 of non-human mammals.

The term “human PD-L1” as used herein includes any variants, isoforms and species homologs of human PD-L1 (UniProt Q9NZQ7), regardless of its source or mode of preparation. Thus, “human PD-L1” includes human PD-L1 naturally expressed by cells and PD-L1 expressed on cells transfected with the human PD-L1 gene.

The terms “anti-PD-L1 antibody,” “PD-L1 antibody,” “anti-PD-L1 binding compound” and “PD-L1 binding compound” are used herein interchangeably to refer to an antibody or binding compound as herein defined, immunospecifically binding to PD-L1, including human PD-L1, as herein defined.

The term “4-1BB” refers to a 4-1BB protein of any human and non-human animal species, and specifically includes human 4-1BB as well as 4-1BB of non-human mammals.

The term “human 4-1BB” as used herein includes any variants, isoforms and species homologs of human 4-1BB (UniProt Q07011), regardless of its source or mode of preparation. Thus, “human 4-1BB” includes human 4-1BB naturally expressed by cells and 4-1BB expressed on cells transfected with the human 4-1BB gene.

The terms “anti-4-1BB antibody,” “4-1BB antibody,” “anti-4-1BB binding compound” and “4-1BB binding compound” are used herein interchangeably to refer to an antibody or binding compound as herein defined, immunospecifically binding to 4-1BB, including human 4-1BB, as herein defined.

The terms “CD47” and “leukocyte surface antigen CD47” refer to a CD47 protein of any human and non-human animal species, and specifically includes human CD47 as well as CD47 of non-human mammals.

The term “human CD47” as used herein includes any variants, isoforms and species homologs of human CD47 (UniProt Q08722), regardless of its source or mode of preparation. Thus, “human CD47” includes human CD47 naturally expressed by cells and CD47 expressed on cells transfected with the human CD47 gene.

The terms “anti-CD47 antibody,” “CD47 antibody,” “anti-CD47 binding compound” and “CD47 binding compound” are used herein interchangeably to refer to an antibody or binding compound as herein defined, immunospecifically binding to CD47, including human CD47, as herein defined.

The terms “IL15” and “interleukin-15” refer to an IL15 protein of any human and non-human animal species, and specifically includes human IL15 as well as IL15 of non-human mammals.

The term “human IL15” as used herein includes any variants, isoforms and species homologs of human IL15 (UniProt P40933), regardless of its source or mode of preparation. Thus, “human IL15” includes human IL15 naturally expressed by cells and IL15 expressed on cells transfected with the human IL15 gene.

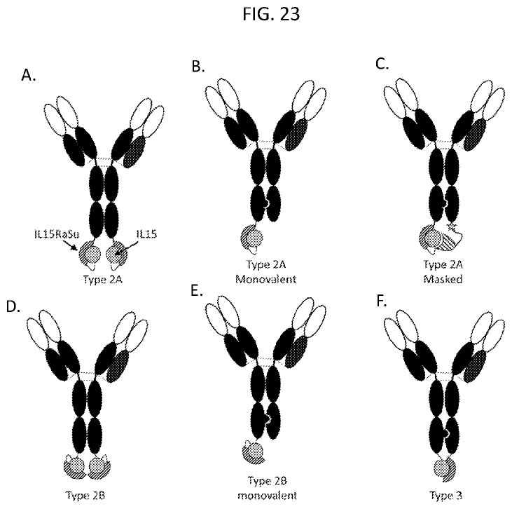

As used herein to describe a multispecific antibody or multispecific binding compound, the term “IL15” refers to an antibody or binding compound comprising a polypeptide subunit (e.g., an antibody heavy chain or an antibody light chain) to which an IL15 protein sequence has been fused, thereby facilitating interaction between the fused IL15 protein and an IL15 receptor, as shown schematically in FIG. 23, panels A-F.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

An “isolated” antibody or binding compound is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

Binding compounds of the invention include multi-specific binding compounds. Multi-specific binding compounds have more than one binding specificity. The term “multi-specific” specifically includes “bispecific” and “trispecific,” as well as higher-order independent specific binding affinities, such as higher-order polyepitopic specificity, as well as tetravalent antibodies and antibody fragments. The terms “multi-specific antibody” and “multi-specific binding compound” are used herein in the broadest sense and cover all antibodies and antibody-like molecules with more than one binding specificity. The multi-specific anti-PD-L1 binding compounds of the present invention specifically include binding compounds immunospecifically binding to an epitope on a PD-L1 protein, such as a human PD-L1 protein, and to an epitope on a different protein, such as, for example, a 4-1BB protein or a CD47 protein.

An “epitope” is the site on the surface of an antigen molecule to which a single antibody molecule binds. Generally, an antigen has several or many different epitopes and reacts with many different antibodies. The term specifically includes linear epitopes and conformational epitopes.

Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure.

The term “valent” as used herein refers to a specified number of binding sites in an antibody molecule or binding compound.

A “monovalent” binding compound has one binding site. Thus, a monovalent binding compound is also monospecific.

A “multi-valent” binding compound has two or more binding sites. Thus, the terms “bivalent”, “trivalent”, and “tetravalent” refer to the presence of two binding sites, three binding sites, and four binding sites, respectively. Thus, a bispecific binding compound according to the invention is at least bivalent and may be trivalent, tetravalent, or otherwise multi-valent. A bivalent binding compound in accordance with embodiments of the invention may have two binding sites to the same epitope (i.e., bivalent, monoparatopic), or to two different epitopes (i.e., bivalent, biparatopic).

A large variety of methods and protein configurations are known and used for the preparation of bispecific monoclonal antibodies (BsMAB) and binding compounds, tri-specific antibodies and binding compounds, and the like.

The term “human antibody” is used herein to include antibodies having variable and constant regions derived from human germline immunoglobulin sequences. The human antibodies herein may include amino acid residues not encoded by human germline immunoglobulin sequences, e.g., mutations introduced by random or site-specific mutagenesis in vitro or by somatic mutation in vivo. The term “human antibody” specifically includes antibodies and binding compounds having human heavy chain variable region sequences.

The term “chimeric” antibody as used herein refers to an antibody having variable sequences derived from a non-human immunoglobulin, such as a rat or a mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816,397, which are incorporated herein by reference in their entireties. The term “chimeric antibody” specifically includes antibodies and binding compounds having variable region sequences derived from a non-human immunoglobulin, and human immunoglobulin constant region sequences.

The term “humanized antibody” as used herein refers to an antibody or binding compound that contains minimal sequences derived from a non-human immunoglobulin. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin sequence. A humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332, all of which are incorporated herein by reference in their entireties.

As used herein, the term “effector cell” refers to an immune cell which is involved in the effector phase of an immune response, as opposed to the cognitive and activation phases of an immune response. Some effector cells express specific Fc receptors and carry out specific immune functions. In some embodiments, an effector cell such as a natural killer cell is capable of inducing antibody-dependent cellular cytotoxicity (ADCC). For example, monocytes and macrophages, which express FcR, are involved in specific killing of target cells and presenting antigens to other components of the immune system, or binding to cells that present antigens. In some embodiments, an effector cell may phagocytose a target antigen or target cell.

“Human effector cells” are leukocytes which express receptors such as T cell receptors or FcRs and perform effector functions. Preferably, the cells express at least Fc⋅RIII and perform ADCC effector function. Examples of human leukocytes which mediate ADCC include natural killer (NK) cells, monocytes, cytotoxic T cells and neutrophils; with NK cells being preferred. The effector cells may be isolated from a native source thereof, e.g., from blood or PBMCs as described herein.

The term “immune cell” is used herein in the broadest sense, including, without limitation, cells of myeloid or lymphoid origin, for instance lymphocytes (such as B cells and T cells including cytolytic T cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express Fc⋅RIII only, whereas monocytes express Fc⋅RI, Fc⋅RII and Fc⋅RIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g., as described in Gazzano-Santoro et al., J. Immunol. Methods 202:163 (1996), may be performed.

“Directed T-cell mediated cytotoxicity” and “re-directed T-cell mediated cytotoxicity”, as used interchangeably herein, refer to a cell-mediated reaction in which a cross-linking molecule (e.g., a bispecific antibody) crosslinks a surface antigen on a T-cell (e.g., CD3) and an antigen on a target cell (e.g., a surface antigen on a cancer cell). Crosslinking of the T-cell and the target cell facilitates killing of the target cell by the T-cell via cytotoxic activity of the T-cell. Re-directed T-cell mediated cytotoxicity is described, for example, in Velasquez et al., Blood 2018 131: 30-38.

“Binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the equilibrium dissociation constant (KD). Affinity can be measured by common methods known in the art. Low-affinity antibodies generally bind antigen slowly and tend to dissociate readily, whereas high-affinity antibodies generally bind antigen faster and tend to remain bound.

As used herein, the “KD” or “KD value” refers to a dissociation constant determined by BioLayer Interferometry, using an Octet Red96 instrument (Fortebio Inc., Menlo Park, CA) in kinetics mode. For example, anti-mouse Fc sensors are loaded with mouse-Fc fused antigen and then dipped into antibody-containing wells to measure concentration dependent association rates (kon). Antibody dissociation rates (koff) are measured in the final step, where the sensors are dipped into wells containing buffer only. The KD is the ratio of koff/kon. (For further details see, Concepcion, J, et al., Comb Chem High Throughput Screen, 12(8), 791-800, 2009).

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), skin cancer, melanoma, lung cancer, including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer (e.g., pancreatic ductal adenocarcinoma), glioblastoma, cervical cancer, ovarian cancer (e.g., high grade serous ovarian carcinoma), liver cancer (e.g., hepatocellular carcinoma (HCC)), bladder cancer (e.g., urothelial bladder cancer), testicular (germ cell tumor) cancer, hepatoma, breast cancer, brain cancer (e.g., astrocytoma), colon cancer, rectal cancer, colorectal cancer, endometrial or uterine carcinoma, salivary gland carcinoma, kidney or renal cancer (e.g., renal cell carcinoma, nephroblastoma or Wilms' tumour), prostate cancer, vulval cancer, thyroid cancer, hepatic carcinoma, anal carcinoma, penile carcinoma, as well as head and neck cancer. Additional examples of cancer include, without limitation, retinoblastoma, thecomas, arrhenoblastomas, hepatoma, hematologic malignancies including non-Hodgkin's lymphoma (NHL), multiple myeloma and acute hematologic malignancies, endometrial or uterine carcinoma, endometriosis, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, melanoma, skin carcinomas, Schwannoma, oligodendroglioma, neuroblastomas, rhabdomyosarcoma, osteogenic sarcoma, leiomyosarcomas, and urinary tract carcinomas.

The term “metastatic cancer” means the state of cancer where the cancer cells of a tissue of origin are transmitted from the original site to one or more sites elsewhere in the body, by the blood vessels or lymphatics, to form one or more secondary tumors in one or more organs besides the tissue of origin. A prominent example is metastatic breast cancer.

The term “characterized by expression of PD-L1” broadly refers to any disease or disorder in which PD-L1 expression is associated with or involved with one or more pathological processes that are characteristic of the disease or disorder. Specifically, and without limitation, a disease or disorder that is characterized by expression of PD-L1 includes, e.g., a cancer in which tumor cells express PD-L1, and/or tumor-associated stroma exhibits expression of PD-L1, and/or PD-L1 is expressed on immune cells. Such disorders include, but are not limited to: invasive breast carcinoma, colon adenocarcinoma, lymphomas, lymphoid neoplasm diffuse large B-cell lymphoma, esophageal carcinoma, head and neck squamous cell carcinoma, lung adenocarcinoma, lung squamous cell carcinoma, ovarian serous cystadenocarcinoma, pancreatic adenocarcinoma, rectum adenocarcinoma, bladder urothelial carcinoma, cervical squamous cell carcinoma and endocervical adenocarcinoma, cholangio carcinoma, glioblastoma multiforme, hepatocellular carcinoma, mesothelioma, merkel cell carcinoma, renal cell carcinoma, sarcoma (e.g., undifferentiated sarcoma), skin cutaneous melanoma, stomach adenocarcinoma, testicular germ cell tumors, uterine carcinosarcoma, osteosarcoma, glioblastoma, melanoma, ovarian, gastric, and colorectal cancers.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues.

The terms “treat”, “treatment” or “treating” as used herein refer to both therapeutic treatment and prophylactic of preventative measures, wherein the object is to prevent or slow down (lessen) a targeted pathological condition or disorder. A subject in need of treatment includes a subject already having a particular condition or disorder, as well as a subject prone to having the disorder or a subject in whom the disorder is to be prevented.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer, individuals with autoimmune diseases, with pathogen infections, and the like. Subjects may be human, but also include other mammals, particularly those mammals useful as laboratory models for human disease, e.g., mouse, rat, etc.

The term “pharmaceutical formulation” refers to a preparation which is in such form as to permit the biological activity of the active ingredient to be effective, and which contains no additional components which are unacceptably toxic to a subject to which the formulation would be administered. Such formulations are sterile. “Pharmaceutically acceptable” excipients (vehicles, additives) are those which can reasonably be administered to a subject mammal to provide an effective dose of the active ingredient employed.

A “sterile” formulation is aseptic or free or essentially free from all living microorganisms and their spores. A “frozen” formulation is one at a temperature below 0° C.

A “stable” formulation is one in which the protein therein essentially retains its physical stability and/or chemical stability and/or biological activity upon storage. Preferably, the formulation essentially retains its physical and chemical stability, as well as its biological activity upon storage. The storage period is generally selected based on the intended shelf-life of the formulation. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29-90) (1993), for example. Stability can be measured at a selected temperature for a selected time period. Stability can be evaluated qualitatively and/or quantitatively in a variety of different ways, including evaluation of aggregate formation (for example using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g., Asn deamidation), oxidation (e.g., Met oxidation), isomerization (e.g., Asp isomerisation), clipping/hydrolysis/fragmentation (e.g., hinge region fragmentation), succinimide formation, unpaired cysteine(s), N-terminal extension, C-terminal processing, glycosylation differences, etc.

### II. Detailed Description

### PD-L1×4-1BB Bispecific Antibodies

Aspects of the invention include multispecific binding compounds, e.g., bispecific antibodies, that bind to PD-L1 and 4-1BB. The multispecific binding compounds can comprise various configurations, and each binding unit can comprise a set of CDR sequences. PD-L1 heavy chain CDR sequences include SEQ ID NOs: 1-6. PD-L1 light chain CDR sequences include SEQ ID NOs: 7-12. Anti-4-1BB heavy chain CDR sequences include SEQ ID NOs: 13-18, and anti-4-1BB light chain CDR sequences include SEQ ID NOs: 19-24. In some embodiments, a multispecific binding compound comprises a CDR sequence with two or fewer amino acid substitutions in any one SEQ ID NOs: 1-24.

Multispecific binding compounds in accordance with embodiments of the invention can comprise any suitable combination of heavy chain and light chain variable region sequences, as provided herein. Anti-PD-L1 heavy chain variable region sequences include SEQ ID NOs: 25 and 26. Anti-PD-L1 light chain variable region sequences include SEQ ID NOs: 27-28. Anti-4-1BB heavy chain variable region sequences include SEQ ID NOs: 29, 30, 45 and 46. Anti-4-1BB light chain variable region sequences include SEQ ID NOs: 31, 32, 47 and 48. In some embodiments, a multispecific binding compound comprises a variable region sequence having at least about 80% identity, such as about 85%, about 90%, about 95%, about 99%, or about 99.9% identity to a variable region sequence of any one of SEQ ID NOs: 25-32 and 45-48.

Multispecific binding compounds in accordance with embodiments of the invention can comprise one or more anti-4-1BB scFv sequences, as listed herein. Anti-4-1BB scFv sequences include SEQ ID NOs: 129-132. In some embodiments, a multispecific binding compound comprises an scFv sequence having at least about 80% identity, such as about 85%, about 90%, about 95%, about 99%, or about 99.9% identity to an scFv sequence of any one of SEQ ID NOs: 129-132. In some embodiments, an scFv sequence is linked to a polypeptide subunit (e.g., a heavy chain or a light chain polypeptide subunit) by a linker sequence. Non-limiting examples of linker sequences include SEQ ID NOs: 36, 37, 38, 49 and 120-128.

The multispecific binding compounds described herein provide a number of benefits that contribute to utility as clinically therapeutic agent(s). The multispecific binding compounds include members with a variety of binding unit configurations, allowing the selection of a specific molecule that shows therapeutic benefits.

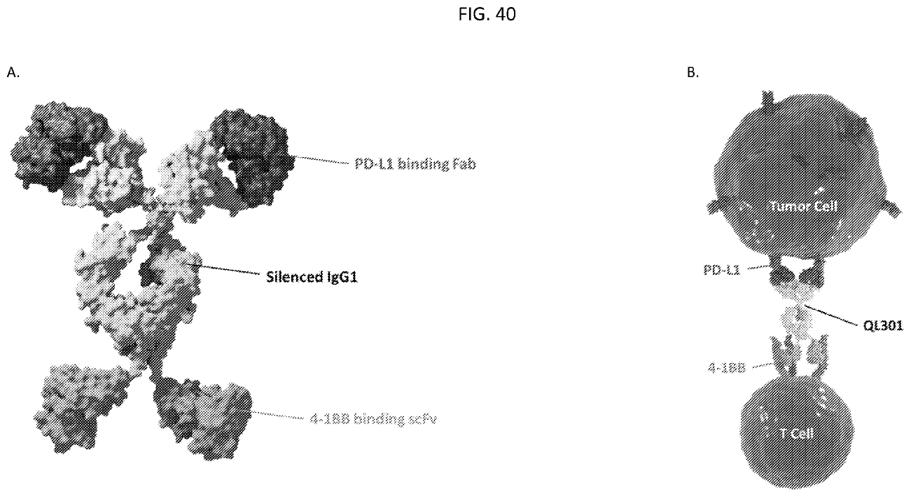

A suitable binding compound may be selected from those provided herein for development and therapeutic or other use, including, without limitation, use as a bispecific binding compound, e.g., as shown in FIG. 40, panel A.

In one preferred embodiment, a bispecific binding compound binds to PD-L1 and 4-1BB, and comprises a first light chain polypeptide comprising the sequence of SEQ ID NO: 43, a first heavy chain polypeptide comprising the sequence of SEQ ID NO: 41, a second heavy chain polypeptide comprising the sequence of SEQ ID NO: 41, and a second light chain polypeptide comprising the sequence of SEQ ID NO: 43. This bispecific antibody is referred to as QL301 (with signal sequence).

In one preferred embodiment, a bispecific binding compound binds to PD-L1 and 4-1BB, and comprises a first light chain polypeptide comprising the sequence of SEQ ID NO: 44, a first heavy chain polypeptide comprising the sequence of SEQ ID NO: 42, a second heavy chain polypeptide comprising the sequence of SEQ ID NO: 42, and a second light chain polypeptide comprising the sequence of SEQ ID NO: 44. This bispecific antibody is referred to as QL301 (without signal sequence).

Determination of affinity for a candidate protein can be performed using methods known in the art, such as Biacore measurements. Multispecific binding compounds as described herein may have an affinity for PD-L1 or 4-1BB with a Kd of from about 10⁻⁶ to around about 10⁻¹¹, including without limitation: from about 10⁻⁶ to around about 10⁻¹⁰; from about 10⁻⁶ to around about 10⁻⁹; from about 10⁻⁶ to around about 10⁻⁸; from about 10⁻⁸ to around about 10⁻¹¹; from about 10⁻⁸ to around about 10⁻¹⁰; from about 108 to around about 10⁻⁹; from about 10⁻⁹ to around about 10⁻¹¹; from about 10⁻⁹ to around about 10⁻¹⁰; or any value within these ranges. The affinity selection may be confirmed with a biological assessment for modulating a PD-L1 or 4-1BB biological activity, including in vitro assays, pre-clinical models, and clinical trials, as well as assessment of potential toxicity.

Various formats of multispecific binding compounds are within the ambit of the invention, including, without limitation, four chain polypeptides, as described herein. The multispecific binding compounds herein specifically include bispecific binding compounds having binding affinity to PD-L1 and 4-1BB (e.g., anti-PD-L1×anti-4-1BB binding compounds). Such bispecific binding compounds induce potent T-cell mediated killing of tumor cells, as depicted in FIG. 40, panel B.

### PD-L1×CD47 Bispecific Antibodies

Aspects of the invention include multispecific binding compounds, e.g., bispecific antibodies, that bind to PD-L1 and CD47. The multispecific binding compounds can comprise various configurations, and each binding unit can comprise a set of CDR sequences. PD-L1 heavy chain CDR sequences include SEQ ID NOs: 1-6. PD-L1 light chain CDR sequences include SEQ ID NOs: 7-12. Anti-CD47 heavy chain CDR sequences include SEQ ID NOs: 50-55, and anti-CD47 light chain CDR sequences include SEQ ID NOs: 56-61. In some embodiments, a multispecific binding compound comprises a CDR sequence with two or fewer amino acid substitutions in any one SEQ ID NOs: 1-12 or 50-61.

Multispecific binding compounds in accordance with embodiments of the invention can comprise any suitable combination of heavy chain and light chain variable region sequences, as provided herein. Anti-PD-L1 heavy chain variable region sequences include SEQ ID NOs: 25 and 26. Anti-PD-L1 light chain variable region sequences include SEQ ID NOs: 27-28. Anti-CD47 heavy chain variable region sequences include SEQ ID NOs: 62-63. Anti-CD47 light chain variable region sequences include SEQ ID NOs: 64-65. In some embodiments, a multispecific binding compound comprises a variable region sequence having at least about 80% identity, such as about 85%, about 90%, about 95%, about 99%, or about 99.9% identity to a variable region sequence of any one of SEQ ID NOs: 25-28 and 62-65.

Multispecific binding compounds in accordance with embodiments of the invention can comprise one or more anti-CD47 scFv sequences, as listed herein. Anti-CD47 scFv sequences include SEQ ID NOs: 72-75. In some embodiments, a multispecific binding compound comprises an scFv sequence having at least about 80% identity, such as about 85%, about 90%, about 95%, about 99%, or about 99.9% identity to an scFv sequence of any one of SEQ ID NOs: 72-75. In some embodiments, an scFv sequence is linked to a polypeptide subunit (e.g., a heavy chain or a light chain polypeptide subunit) by a linker sequence. Non-limiting examples of linker sequences include SEQ ID NOs: 36, 37, 38, 49 and 120-128.

The multispecific binding compounds described herein provide a number of benefits that contribute to utility as clinically therapeutic agent(s). The multispecific binding compounds include members with a variety of binding unit configurations, allowing the selection of a specific molecule that shows therapeutic benefits.

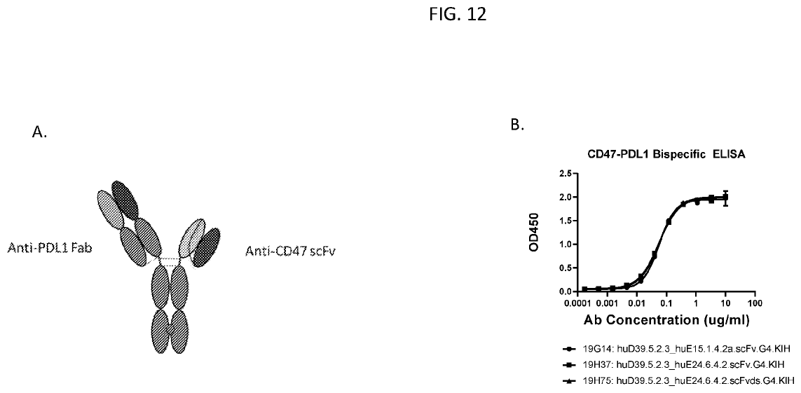

A suitable binding compound may be selected from those provided herein for development and therapeutic or other use, including, without limitation, use as a bispecific binding compound, e.g., as shown in FIG. 12, panel A.

Aspects of the invention include multispecific binding compounds that comprise a knobs-into-holes (KiH) interface between their heavy chain subunits to facilitate heterodimerization of the desired components of the multispecific compound, e.g., a first heavy chain polypeptide subunit comprising an anti-PD-L1 binding domain, and a second heavy chain polypeptide subunit comprising an anti-CD47 binding domain (e.g., an anti-CD47 scFv).

In one preferred embodiment, a bispecific binding compound binds to PD-L1 and CD47, and comprises a first light chain polypeptide comprising the sequence of SEQ ID NO: 66, a first heavy chain polypeptide comprising the sequence of SEQ ID NO: 67, a second heavy chain polypeptide comprising the sequence of SEQ ID NO: 68. This molecule is referred to as huD39.5.2.3-huG4a_hole_RF_huE15.1_scFvds-huG4a_hingeFc_knob_KiHss.

In one preferred embodiment, a bispecific binding compound binds to PD-L1 and CD47, and comprises a first light chain polypeptide comprising the sequence of SEQ ID NO: 69, a first heavy chain polypeptide comprising the sequence of SEQ ID NO: 70, a second heavy chain polypeptide comprising the sequence of SEQ ID NO: 71. This molecule is referred to as huD39.5.2.3-huG4a_hole_RF_huE24.6_scFvds-huG4a_hingeFc_knob_KiHss.

Determination of affinity for a candidate protein can be performed using methods known in the art, such as Biacore measurements. Multispecific binding compounds as described herein may have an affinity for PD-L1 or CD47 with a Kd of from about 10⁻⁶ to around about 10⁻¹¹, including without limitation: from about 10⁻⁶ to around about 10⁻¹⁰; from about 10⁻⁶ to around about 10⁻⁹; from about 10⁻⁶ to around about 10⁻⁸; from about 10⁻⁸ to around about 10⁻¹¹; from about 10⁻⁸ to around about 10⁻¹⁰; from about 10⁻⁸ to around about 10⁻⁹; from about 10⁻⁹ to around about 10⁻¹¹; from about 10⁻⁹ to around about 10⁻¹⁰; or any value within these ranges. The affinity selection may be confirmed with a biological assessment for modulating a PD-L1 or CD47 biological activity, including in vitro assays, pre-clinical models, and clinical trials, as well as assessment of potential toxicity.

Various formats of multispecific binding compounds are within the ambit of the invention, including, without limitation, three chain or four chain polypeptides, as described herein. The multispecific binding compounds herein specifically include bispecific binding compounds having binding affinity to PD-L1 and CD47 (e.g., anti-PD-L1×anti-CD47 binding compounds). Such bispecific binding compounds induce potent T-cell mediated killing of tumor cells.

### PD-L1×IL15 Binding Compounds

Aspects of the invention include multispecific binding compounds, e.g., bispecific antibodies, that bind to PD-L1 and comprise an IL15 region that facilitates interaction with an IL15 receptor. The multispecific binding compounds can comprise various configurations, and each PD-L1 binding unit can comprise a set of CDR sequences. PD-L1 heavy chain CDR sequences include SEQ ID NOs: 1-6. PD-L1 light chain CDR sequences include SEQ ID NOs: 7-12. In some embodiments, a multispecific binding compound comprises a CDR sequence with two or fewer amino acid substitutions in any one SEQ ID NOs: 1-12.

Multispecific binding compounds in accordance with embodiments of the invention can comprise any suitable combination of heavy chain and light chain variable region sequences, as provided herein. Anti-PD-L1 heavy chain variable region sequences include SEQ ID NOs: 25 and 26. Anti-PD-L1 light chain variable region sequences include SEQ ID NOs: 27-28. In some embodiments, a multispecific binding compound comprises a variable region sequence having at least about 80% identity, such as about 85%, about 90%, about 95%, about 99%, or about 99.9% identity to a variable region sequence of any one of SEQ ID NOs: 25-28.

Multispecific binding compounds in accordance with embodiments of the invention can comprise one or more IL15 sequences, as listed herein. IL15 sequences include SEQ ID NOs: 86-90. In some embodiments, a multispecific binding compound comprises an IL15 sequence having at least about 80% identity, such as about 85%, about 90%, about 95%, about 99%, or about 99.9% identity to an IL15 sequence of any one of SEQ ID NOs: 86-90. In some embodiments, an IL15 sequence is linked to a polypeptide subunit (e.g., a heavy chain or a light chain polypeptide subunit) by a linker sequence. Non-limiting examples of linker sequences include SEQ ID NOs: 36, 37, 38, 49 and 120-128.

The multispecific binding compounds described herein provide a number of benefits that contribute to utility as clinically therapeutic agent(s). The multispecific binding compounds include members with a variety of binding unit configurations, allowing the selection of a specific molecule that shows therapeutic benefits.

A suitable binding compound may be selected from those provided herein for development and therapeutic or other use, including, without limitation, use as a bispecific binding compound, e.g., as shown in FIG. 23, panels A-F.

Aspects of the invention include multispecific binding compounds that comprise a knobs-into-holes (KiH) interface between their heavy chain subunits to facilitate heterodimerization of the desired components of the multispecific compound, e.g., a first heavy chain polypeptide subunit comprising an anti-PD-L1 binding domain and first IL15 protein, and a second heavy chain polypeptide subunit comprising an anti-PD-L1 binding domain and a second IL15 protein.

In one preferred embodiment, a bispecific binding compound binds to PD-L1 and comprises two IL15 proteins, one on each heavy chain polypeptide subunit, and comprises a first light chain polypeptide comprising the sequence of SEQ ID NO: 104, a first heavy chain polypeptide comprising the sequence of SEQ ID NO: 105, a second heavy chain polypeptide comprising the sequence of SEQ ID NO: 105, and a second light chain polypeptide comprising the sequence of SEQ ID NO: 104. This molecule is referred to as D39.5.2.3-G1AAA-IL15RaSu-IL15-T2A, and is depicted schematically in FIG. 23, panel A.

In one preferred embodiment, a bispecific binding compound binds to PD-L1 and comprises two IL15 proteins, one on each heavy chain polypeptide subunit, and comprises a first light chain polypeptide comprising the sequence of SEQ ID NO: 106, a first heavy chain polypeptide comprising the sequence of SEQ ID NO: 107, a second heavy chain polypeptide comprising the sequence of SEQ ID NO: 107, and a second light chain polypeptide comprising the sequence of SEQ ID NO: 106. This molecule is referred to as D39.5.2.3-G1AAA-IL15-IL15 RaSu-T2B, and is depicted schematically in FIG. 23, panel D.

In one preferred embodiment, a bispecific binding compound binds to PD-L1 and comprises two IL15 proteins, one on each heavy chain polypeptide subunit, and comprises a first and a second light chain polypeptide comprising the sequence of SEQ ID NO: 108, a first heavy chain polypeptide comprising the sequence of SEQ ID NO: 109, and a second heavy chain polypeptide comprising the sequence of SEQ ID NO: 110. This molecule is a four chain molecule, and comprises a KiH interface between the heavy chain polypeptides to facilitate heterodimerization. This molecule is referred to as D39.5.2.3-G1AAA.KiH-IL15+IL15RaSu-T3, and is depicted schematically in FIG. 23, panel F.

In one preferred embodiment, a bispecific binding compound binds to PD-L1 and comprises one IL15 protein, on one heavy chain polypeptide subunit, and comprises a first and a second light chain polypeptide comprising the sequence of SEQ ID NO: 111, a first heavy chain polypeptide comprising the sequence of SEQ ID NO: 112, and a second heavy chain polypeptide comprising the sequence of SEQ ID NO: 113. This molecule is a four chain molecule, and comprises a KiH interface between the heavy chain polypeptides to facilitate heterodimerization. This molecule is referred to as D39.5.2.3-G1AAA-IL15RaSu-IL15-T2A-mono, and is depicted schematically in FIG. 23, panel B.

In one preferred embodiment, a bispecific binding compound binds to PD-L1 and comprises one IL15 protein, on one heavy chain polypeptide subunit, and comprises a first and a second light chain polypeptide comprising the sequence of SEQ ID NO: 114, a first heavy chain polypeptide comprising the sequence of SEQ ID NO: 115, and a second heavy chain polypeptide comprising the sequence of SEQ ID NO: 116. This molecule is a four chain molecule, and comprises a KiH interface between the heavy chain polypeptides to facilitate heterodimerization. This molecule is referred to as D39.5.2.3-G1AAA-IL15-IL15RaSu-T2B-mono, and is depicted schematically in FIG. 23, panel E.

In one preferred embodiment, a bispecific binding compound binds to PD-L1 and comprises two IL15 proteins, one on each heavy chain polypeptide subunit, and comprises a first light chain polypeptide comprising the sequence of SEQ ID NO: 117, a first heavy chain polypeptide comprising the sequence of SEQ ID NO: 118, and a second heavy chain polypeptide comprising the sequence of SEQ ID NO: 119, and a second light chain polypeptide comprising the sequence of SEQ ID NO: 117. This molecule is a four chain molecule, and comprises a KiH interface between the heavy chain polypeptides to facilitate heterodimerization. This molecule is referred to as D39.5.2.3-G1AAA-IL15RaSu-IL15-T2A-masked, and is depicted schematically in FIG. 23, panel C.

Determination of affinity for a candidate protein can be performed using methods known in the art, such as Biacore measurements. Multispecific binding compounds as described herein may have an affinity for PD-L1 with a Kd of from about 10⁻⁶ to around about 10⁻¹¹, including without limitation: from about 10⁻⁶ to around 10⁻¹⁰; from about 10⁻⁶ around about 10⁻⁹; from about 10⁻⁶ around about 10⁻⁸; from about 10⁻⁸ to around about 10⁻¹¹; from about 10⁻⁸ to around about 10⁻¹⁰; from about 10⁻⁸ to around about 10⁻⁹; from about 10⁻⁹ to around about 10⁻¹¹; from about 10⁻⁹ to around about 10⁻¹⁰; or any value within these ranges. The affinity selection may be confirmed with a biological assessment for modulating a PD-L1 or IL15 biological activity, including in vitro assays, pre-clinical models, and clinical trials, as well as assessment of potential toxicity.

Various formats of multispecific binding compounds are within the ambit of the invention, including, without limitation, three chain or four chain polypeptides, as described herein. The multispecific binding compounds herein specifically include bispecific binding compounds having binding affinity to PD-L1 and comprising one or more IL15 proteins that facilitate interaction with an IL15 receptor (e.g., anti-PD-L1×IL15 binding compounds). Such bispecific binding compounds induce potent T-cell mediated killing of tumor cells.

The tables below provide various sequences that are used in assembling the binding compounds described herein.

### PD-L1 Heavy Chain CDR Sequences:

| Clone ID: | HCDR1 | HCDR2 | HCDR3 |
|---|---|---|---|
| D39.5 | TFWMH (SEQ ID | NIYPGSGTINYDEKFRS | GWDGEH (SEQ ID |
| | NO: 1) | (SEQ ID NO: 2) | NO: 3) |
| C44.1 | DYGMH (SEQ ID | YIGTTSSIIYYADTVKG | RDYGNYYWYLDV |
| | NO: 4) | (SEQ ID NO: 5) | (SEQ ID NO: 6) |

### PD-L1 Light Chain CDR Sequences:

| Clone ID: | LCDR1 | LCDR2 | LCDR3 |
|---|---|---|---|
| D39.5 | RASENIHSNLA (SEQ | GATNLAD (SEQ ID | QHFWGTPPYA (SEQ |
| | ID NO: 7) | NO: 8) | ID NO: 9) |
| C44.1 | SASSSVEDMY (SEQ | RTSNLAS (SEQ ID | QQYQSFPLT (SEQ ID |
| | ID NO: 10) | NO: 11) | NO: 12) |

### 4-1BB Heavy Chain CDR Sequences:

| Clone ID: | HCDR1 | HCDR2 | HCDR3 |
|---|---|---|---|
| F1.1 | FYTMH (SEQ ID | YINPSSGYTNYNQKFTD | SDGSSSKWYFDV |
| | NO: 13) | (SEQ ID NO: 14) | (SEQ ID NO: 15) |
| G28.21 | DYYIH (SEQ ID NO: | RIDPEDGDIAYAPKFQD | GNYYAMDF (SEQ |
| | 16) | (SEQ ID NO: 17) | ID NO: 18) |

### 4-1BB Light Chain CDR Sequences:

| Clone ID: | LCDR1 | LCDR2 | LCDR3 |
|---|---|---|---|
| F1.1 | RASSSVSYIH (SEQ | ATSNLAS (SEQ ID | QQWSSNPFT (SEQ |
| | ID NO: 19) | NO: 20) | ID NO: 21) |
| G28.21 | TASSSVSSSYLH | STSNLAS (SEQ ID | HQYHRSPPT (SEQ ID |
| | (SEQ ID NO: 22) | NO: 23) | NO: 24) |

### CD47 Heavy Chain CDR Sequences:

| Clone ID: | HCDR1 | HCDR2 | HCDR3 |
|---|---|---|---|
| E15.1 | GYYMN (SEQ ID | DLNPDNGDTNYNQKFVG | GGKGGFDY (SEQ ID |
| | NO: 50) | (SEQ ID NO: 51) | NO: 52) |
| E24.6 | DYYIN (SEQ ID | WIYPGSGNTKYNEKFKD | KGIIYNYGSSDVLAY |
| | NO: 53) | (SEQ ID NO: 54) | (SEQ ID NO: 55) |

### CD47 Light Chain CDR Sequences:

| Clone ID: | LCDR1 | LCDR2 | LCDR3 |
|---|---|---|---|
| E15.1 | RSSQSLEKSNGNTYLN | RVSRRYS (SEQ ID | LQVTHVPYT (SEQ |
| | (SEQ ID NO: 56) | NO: 57) | ID NO: 58) |
| E24.6 | KSSQSLLYSSNQKNYLA | WASTRES (SEQ ID | HQYYSYPLT (SEQ |
| | (SEQ ID NO: 59) | NO: 60) | ID NO: 61) |

### PD-L1 Heavy Chain Variable Region Sequences:

| Clone ID: | Heavy chain variable region sequence: |
|---|---|
| D39.5 | QVQLVQSGAEVVKPGASVKLSCKASGYTFTTFWMHWVRQAPGQGLEWIGNIY |
| | PGSGTINYDEKFRSRATLTVDTSISTAYMEVSRLRSEDTAVYYCTTGWDGEHW |
| | GQGTTLTVSS (SEQ ID NO: 25) |
| C44.1 | EVQLVESGGGLVKPGGSLRLSCAASGFTFSDYGMHWIRQAPGKGLEWIAYIGT |
| | TSSIIYYADTVKGRFTISRDNAKNSLYLQMNSLRAEDTAVYYCARRDYGNYYW |
| | YLDVWGTGTMVTVSS (SEQ ID NO: 26) |

### PD-L1 Light Chain Variable Region Sequences:

| Clone ID: | Light chain variable region sequence: |
|---|---|
| D39.5 | DIQMTQSPSSLSVSVGDRVTITCRASENIHSNLAWYQQKPGKAPQLLVYGATNL |
| | ADGVPSRFSGSGSGAQYTLTISSLQPEDFATYYCQHFWGTPPYAFGGGTKLEIK |
| | (SEQ ID NO: 27) |
| C44.1 | EIVLTQSPATLSLSPGERVTLSCSASSSVFDMYWYQQKPGSSPRPWIYRTSNLAS |
| | GVPARFSGSGSGTDFFLTISSLEPEDAAVYYCQQYQSFPLTFGQGTKLELK (SEQ |
| | ID NO: 28) |

### 4-1BB Heavy Chain Variable Region Sequences:

| Clone ID: | Heavy chain variable region sequence: |
|---|---|
| F1.1 | QVQLVQSGAEVKKPGASVKMSCKASGYTFTFYTMHWLKQAPGQGLEWIGYIN |
| (without | PSSGYTNYNQKFTDRATLTADKSTSTAYMELSSLRSEDTAVYYCARSDGSSSK |
| stabilizing | WYFDVWGQGTTVTVSS (SEQ ID NO: 29) |
| disulfides) | |
| F1.1 (with | QVQLVQSGAEVKKPGASVKMSCKASGYTFTFYTMHWLKQAPGQCLEWIGYIN |
| stabilizing | PSSGYTNYNQKFTDRATLTADKSTSTAYMELSSLRSEDTAVYYCARSDGSSSK |
| disulfides | WYFDVWGQGTTVTVSS (SEQ ID NO: 45) |
| G44C, Q100C) | |
| G28.21 | EVQLVQSGAEVKKPGATVKISCKASGFNIKDYYIHWVNQAPGKGLEWIGRIDP |
| (without | EDGDIAYAPKFQDRVTLTVDTSTDTAYLELSSLRSEDTAVYYCTTGNYYAMDF |
| stabilizing | WGQGTTVTVSS (SEQ ID NO: 30) |
| disulfides) | |
| G28.21 | EVQLVQSGAEVKKPGATVKISCKASGFNIKDYYIHWVNQAPGKCLEWIGRIDPE |
| (with | DGDIAYAPKFQDRVTLTVDTSTDTAYLELSSLRSEDTAVYYCTTGNYYAMDFW |
| stabilizing | GQGTTVTVSS (SEQ ID NO: 46) |
| disulfides | |
| G44C, Q100C) | |

### 4-1BB Light Chain Variable Region Sequences:

| Clone ID: | Light chain variable region sequence: |
|---|---|
| F1.1 | EIVLTQSPDFQSATPKEKVTITCRASSSVSYIHWYQQKPGSSPKAWISATSNLAS |
| (without | GVPSRFSGSGSGTSYTLTINRVEAEDAATYYCQQWSSNPFTFGQGTKLEIK (SEQ |
| stabilizing | ID NO: 31) |
| disulfides) | |
| F1.1 (with | EIVLTQSPDFQSATPKEKVTITCRASSSVSYIHWYQQKPGSSPKAWISATSNLAS |
| stabilizing | GVPSRFSGSGSGTSYTLTINRVEAEDAATYYCQQWSSNPFTFGCGTKLEIK (SEQ |
| disulfides | ID NO: 47) |
| G44C, Q100C) | |
| G28.21 | QIVLTQSPATLSASPGERVTLSCTASSSVSSSYLHWYQQKPGSSPKLWIYSTSNL |
| (without | ASGVPARFSGSGPGTSYTLTISSMEPEDAATYYCHQYHRSPPTFGQGTKLEIK |
| stabilizing | (SEQ ID NO: 32) |
| disulfides) | |
| G28.21 | QIVLTQSPATLSASPGERVTLSCTASSSVSSSYLHWYQQKPGSSPKLWIYSTSNL |
| (with | ASGVPARFSGSGPGTSYTLTISSMEPEDAATYYCHQYHRSPPTFGCGTKLEIK |
| stabilizing | (SEQ ID NO: 48) |
| disulfides | |
| G44C, Q100C) | |

4-1BB scFv Sequences:

| Clone ID: | Light chain variable region sequence: |
|---|---|
| F1.1 scFv with | QVQLVQSGAEVKKPGASVKMSCKASGYTFTFYTMHWLKQAPGQC |
| stabilizing | LEWIGYINPSSGYTNYNQKFTDRATLTADKSTSTAYMELSSLRSEDT |
| disulfide | AVYYCARSDGSSSKWYFDVWGQGTTVTVSSGGGGSGGGGSGGGG |
| bonds, | SEIVLTQSPDFQSATPKEKVTITCRASSSVSYIHWYQQKPGSSPKAWI |
| G44C, Q100C | SATSNLASGVPSRFSGSGSGTSYTLTINRVEAEDAATYYCQQWSSNP |
| | FTFGCGTKLEIK (SEQ ID NO: 129) |
| F1.1 scFv without | QVQLVQSGAEVKKPGASVKMSCKASGYTFTFYTMHWLKQAPGQG |
| stabilizing | LEWIGYINPSSGYTNYNQKFTDRATLTADKSTSTAYMELSSLRSEDT |
| disulfide | AVYYCARSDGSSSKWYFDVWGQGTTVTVSSGGGGSGGGGSGGGG |
| bonds | SEIVLTQSPDFQSATPKEKVTITCRASSSVSYIHWYQQKPGSSPKAWI |
| | SATSNLASGVPSRFSGSGSGTSYTLTINRVEAEDAATYYCQQWSSNP |
| | FTFGQGTKLEIK (SEQ ID NO: 130) |
| G28.21 scFv with | EVQLVQSGAEVKKPGATVKISCKASGFNIKDYYIHWVNQAPGKCL |
| stabilizing | EWIGRIDPEDGDIAYAPKFQDRVTLTVDTSTDTAYLELSSLRSEDTA |
| disulfide | VYYCTTGNYYAMDFWGQGTTVTVSSGGGGSGGGGSGGGGSQIVL |
| bonds, | TQSPATLSASPGERVTLSCTASSSVSSSYLHWYQQKPGSSPKLWIYS |
| G44C, Q100C | TSNLASGVPARFSGSGPGTSYTLTISSMEPEDAATYYCHQYHRSPPT |
| | FGCGTKLEIK (SEQ ID NO: 131) |
| G28.21 scFv without | EVQLVQSGAEVKKPGATVKISCKASGFNIKDYYIHWVNQAPGKGL |
| stabilizing | EWIGRIDPEDGDIAYAPKFQDRVTLTVDTSTDTAYLELSSLRSEDTA |
| disulfide | VYYCTTGNYYAMDFWGQGTTVTVSSGGGGSGGGGSGGGGSQIVL |
| bonds | TQSPATLSASPGERVTLSCTASSSVSSSYLHWYQQKPGSSPKLWIYS |
| | TSNLASGVPARFSGSGPGTSYTLTISSMEPEDAATYYCHQYHRSPPT |
| | FGQGTKLEIK (SEQ ID NO: 132) |

### CD47 Heavy Chain Variable Region Sequences:

| Clone ID: | Heavy chain variable region sequence: |
|---|---|
| E15.1 | EVQLQQSGAEVKKPGASVKISCKASGYTFTGYYMNWMKQSHGKSLEWIGDLN |
| | PDNGDTNYNQKFVGRATLTVDKSISTAYMELSRLRSEDTAVYYCARGGKGGF |
| | DYWGQGTTLTVSS (SEQ ID NO: 62) |
| E24.6 | QIQLQQSGAEVKKPGASVKISCKASGYTFIDYYINWVKQRPGQGLEWIGWIYPG |
| | SGNTKYNEKFKDRGTLTVDTSSSTAYMELSSLRSEDTAVYFCVRKGIIYNYGSS |
| | DVLAYWGQGTLVTVSS (SEQ ID NO: 63) |

CD47 Light chain Variable Region sequence:

| Clone ID: | Light chain variable region sequence: |
|---|---|
| E15.1 | DAVMTQSPLSLPVTLGQPASISCRSSQSLEKSNGNTYLNWYLQRPGQSPQLLIY |
| | RVSRRYSGVPDRFSGSGSGTDFTLKISRVEAEDVGVYYCLQVTHVPYTFGQGTR |
| | LEIK (SEQ ID NO: 64) |
| E24.6 | DIVMTQSPDSLAVSLGERLTINCKSSQSLLYSSNQKNYLAWYQQKPGQSPKLLI |
| | YWASTRESGVPDRFSGSGSGTDFTLTISSVQAEDVAVYYCHQYYSYPLTFGQGT |
| | KLELK (SEQ ID NO: 65) |

CD47 scFv Sequences:

| Name: | Sequence: |
|---|---|
| huE15.1_scFvds | EVQLQQSGAEVKKPGASVKISCKASGYTFTGYYMNWMKQSHGKCLE |
| | WIGDLNPDNGDTNYNQKFVGRATLTVDKSISTAYMELSRLRSEDTAVY |
| | YCARGGKGGFDYWGQGTTLTVSSGGGGSGGGGSGGGGSADVVMTQS |
| | PLSLPVTLGQPASISCRSSQSLEKSNGNTYLNWYLQRPGQSPQLLIYRVS |
| | RRYSGVPDRFSGSGSGTDFTLKISRVEAEDVGVYYCLQVTHVPYTFGCG |
| | TRLEIK (SEQ ID NO: 72) |
| huE15.1_scFv | EVQLQQSGAEVKKPGASVKISCKASGYTFTGYYMNWMKQSHGKSLE |
| | WIGDLNPDNGDTNYNQKFVGRATLTVDKSISTAYMELSRLRSEDTAVY |
| | YCARGGKGGFDYWGQGTTLTVSSGGGGSGGGGSGGGGSADVVMTQS |
| | PLSLPVTLGQPASISCRSSQSLEKSNGNTYLNWYLQRPGQSPQLLIYRVS |
| | RRYSGVPDRFSGSGSGTDFTLKISRVEAEDVGVYYCLQVTHVPYTFGQ |
| | GTRLEIK (SEQ ID NO: 73) |
| huE24.6_scFvds | QIQLQQSGAEVKKPGASVKISCKASGYTFIDYYINWVKQRPGQCLEWIG |
| | WIYPGSGNTKYNEKFKDRGTLTVDTSSSTAYMELSSLRSEDTAVYFCV |
| | RKGIIYNYGSSDVLAYWGQGTLVTVSSGGGGSGGGGSGGGGSADIVMT |
| | QSPDSLAVSLGERLTINCKSSQSLLYSSNQKNYLAWYQQKPGQSPKLLI |
| | YWASTRESGVPDRFSGSGSGTDFTLTISSVQAEDVAVYYCHQYYSYPLT |
| | FGCGTKLELK (SEQ ID NO: 74) |
| huE24.6_scFv | QIQLQQSGAEVKKPGASVKISCKASGYTFIDYYINWVKQRPGQGLEWIG |
| | WIYPGSGNTKYNEKFKDRGTLTVDTSSSTAYMELSSLRSEDTAVYFCV |
| | RKGIIYNYGSSDVLAYWGQGTLVTVSSGGGGSGGGGSGGGGSADIVMT |
| | QSPDSLAVSLGERLTINCKSSQSLLYSSNQKNYLAWYQQKPGQSPKLLI |
| | YWASTRESGVPDRFSGSGSGTDFTLTISSVQAEDVAVYYCHQYYSYPLT |
| | FGQGTKLELK (SEQ ID NO: 75) |

Misc. Additional Sequences:

| Name: | Sequence: |
|---|---|
| Human | RTVAAPSVFIFPPSDEQLKSGTASVVCLLNNFYPREAKVQWKVDNAL |
| kappa light | QSGNSQESVTEQDSKDSTYSLSSTLTLSKADYEKHKVYACEVTHQGL |
| chain | SSPVTKSFNRGEC (SEQ ID NO: 33) |
| constant | |
| region | |
| sequence | |
| Human | ASTKGPSVFPLAPSSKSTSGGTAALGCLVKDYFPEPVTVSWNSGALT |
| IgG1 | SGVHTFPAVLQSSGLYSLSSVVTVPSSSLGTQTYICNVNHKPSNTKV |
| constant | DKKVEPKSCDKTHTCPPCPAPELLGGPSVFLFPPKPKDTLMISRTPEV |
| region | TCVVVDVSHEDPEVKFNWYVDGVEVHNAKTKPREEQYNSTYRVVS |
| sequence | VLTVLHQDWLNGKEYKCKVSNKALPAPIEKTISKAKGQPREPQVYT |
| (wild type, | LPPSRDELTKNQVSLTCLVKGFYPSDIAVEWESNGQPENNYKTTPPVL |
| UniProt No. | DSDGSFFLYSKLTVDKSRWQQGNVFSCSVMHEALHNHYTQKSLSL |
| P01857) | SPGK (SEQ ID NO: 34) |
| Human | ASTKGPSVFPLAPSSKSTSGGTAALGCLVKDYFPEPVTVSWNSGALT |
| IgG1 | SGVHTFPAVLQSSGLYSLSSVVTVPSSSLGTQTYICNVNHKPSNTKVD |
| constant | KKVEPKSCDKTHTCPPCPAPEAAGAPSVFLFPPKPKDTLMISRTPEVTC |
| region | VVVDVSHEDPEVKFNWYVDGVEVHNAKTKPREEQYNSTYRVVSVLT |
| sequence | VLHQDWLNGKEYKCKVSNKALPAPIEKTISKAKGQPREPQVYTLPPSR |
| (terminal | DELTKNQVSLTCLVKGFYPSDIAVEWESNGQPENNYKTTPPVLDSDGS |
| lysine | FFLYSKLTVDKSRWQQGNVFSCSVMHEALHNHYTQKSLSLSPG (SEQ ID NO: |
| removed | 35) |
| and Fc | |
| silencing | |
| mutations | |
| L234A, L235A, | |
| G237A) | |
| Linker 1 | GGGGS (SEQ ID NO: 36) |
| Linker 2 | GGGGSGGGGS (SEQ ID NO: 37) |
| Linker 3 | GGGGSGGGGSGGGGS (SEQ ID NO: 38) |
| Linker 4 | GGGGSGGGGSGGGGSA (SEQ ID NO: 49) |
| Heavy chain | MDMRVPAQLLGLLLLWLRGARC (SEQ ID NO: 39) |
| signal | |
| peptide | |
| Light chain | MRVPAQLLGLLLLWFPGARC (SEQ ID NO: 40) |
| signal | |
| peptide | |

### QL301 Full Length Polypeptide Sequences:

| Name: | Sequence: |
|---|---|
| QL301 full | MDMRVPAQLLGLLLLWLRGARCQVQLVQSGAEVVKPGASVKLSCKA |
| length | SGYTFTTFWMHWVRQAPGQGLEWIGNIYPGSGTINYDEKFRSRATLTV |
| heavy chain | DTSISTAYMEVSRLRSEDTAVYYCTTGWDGEHWGQGTTLTVSSASTKG |
| with signal | PSVFPLAPSSKSTSGGTAALGCLVKDYFPEPVTVSWNSGALTSGVHTFP |
| sequence | AVLQSSGLYSLSSVVTVPSSSLGTQTYICNVNHKPSNTKVDKKVEPKSC |
| | DKTHTCPPCPAPEAAGAPSVFLFPPKPKDTLMISRTPEVTCVVVDVSHE |
| | DPEVKFNWYVDGVEVHNAKTKPREEQYNSTYRVVSVLTVLHQDWLN |
| | GKEYKCKVSNKALPAPIEKTISKAKGQPREPQVYTLPPSRDELTKNQVS |
| | LTCLVKGFYPSDIAVEWESNGQPENNYKTTPPVLDSDGSFFLYSKLTVD |
| | KSRWQQGNVFSCSVMHEALHNHYTQKSLSLSPGGGGGSQVQLVQSGA |
| | EVKKPGASVKMSCKASGYTFTFYTMHWLKQAPGQCLEWIGYINPSSGY |
| | TNYNQKFTDRATLTADKSTSTAYMELSSLRSEDTAVYYCARSDGSSSK |
| | WYFDVWGQGTTVTVSSGGGGSGGGGSGGGGSEIVLTQSPDFQSATPK |
| | EKVTITCRASSSVSYIHWYQQKPGSSPKAWISATSNLASGVPSRFSGSG |
| | SGTSYTLTINRVEAEDAATYYCQQWSSNPFTFGCGTKLEIK (SEQ ID NO: 41) |
| QL301 full | QVQLVQSGAEVVKPGASVKLSCKASGYTFTTFWMHWVRQAPGQG |
| length | LEWIGNIYPGSGTINYDEKFRSRATLTVDTSISTAYMEVSRLRSEDTA |
| heavy chain | VYYCTTGWDGEHWGQGTTLTVSSASTKGPSVFPLAPSSKSTSGGTA |
| without | ALGCLVKDYFPEPVTVSWNSGALTSGVHTFPAVLQSSGLYSLSSVV |
| signal | TVPSSSLGTQTYICNVNHKPSNTKVDKKVEPKSCDKTHTCPPCPAPEA |
| sequence | AGAPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPS |
| | DIAVEWESNGQPENNYKTTPPVLDSDGSFFLYSKLTVDKSRWQQGNV |
| | FSCSVMHEALHNHYTQKSLSLSPGGGGGSQVQLVQSGAEVKKPGASV |
| | KMSCKASGYTFTFYTMHWLKQAPGQCLEWIGYINPSSGYTNYNQKFT |
| | DRATLTADKSTSTAYMELSSLRSEDTAVYYCARSDGSSSKWYFDVW |
| | GQGTTVTVSSGGGGSGGGGSGGGGSEIVLTQSPDFQSATPKEKVTITC |
| | RASSSVSYIHWYQQKPGSSPKAWISATSNLASGVPSRFSGSGSGTSYT |
| | LTINRVEAEDAATYYCQQWSSNPFTFGCGTKLEIK (SEQ ID NO: 42) |
| QL301 full | MRVPAQLLGLLLLWFPGARCDIQMTQSPSSLSVSVGDRVTITCRA |
| length light | SENIHSNLAWYQQKPGKAPQLLVYGATNLADGVPSRFSGSGSGA |
| chain with | QYTLTISSLQPEDFATYYCQHFWGTPPYAFGGGTKLEIKRTVAAP |
| signal | SVFIFPPSDEQLKSGTASVVCLLNNFYPREAKVQWKVDNALQSG |
| sequence | NSQESVTEQDSKDSTYSLSSTLTLSKADYEKHKVYACEVTHQGL |
| | SSPVTKSFNRGEC (SEQ ID NO: 43) |
| QL301 full | DIQMTQSPSSLSVSVGDRVTITCRASENIHSNLAWYQQKPGKAPQLL |
| length light | VYGATNLADGVPSRFSGSGSGAQYTLTISSLQPEDFATYYCQHFWG |
| chain | TPPYAFGGGTKLEIKRTVAAPSVFIFPPSDEQLKSGTASVVCLLNNF |
| without | YPREAKVQWKVDNALQSGNSQESVTEQDSKDSTYSLSSTLTLSKA |
| signal | DYEKHKVYACEVTHQGLSSPVTKSFNRGEC (SEQ ID NO: 44) |
| sequence | |

### CD47 Embodiment Sequences:

| Name: | Sequence: |
|---|---|
| huD39.5.2.3- | DIQMTQSPSSLSVSVGDRVTITCRASENIHSNLA |
| huG4a_hole_RF_ | WYQQKPGKAPQLLVYGATNLADGVPSRFSGSG |
| huE15.1_scFvds- | SGAQYTLTISSLQPEDFATYYCQHFWGTPPYAFG |
| huG4a_hingeFc_knob_ | GGTKLEIKRTVAAPSVFIFPPSDEQLKSGTASVVC |
| KiHss | LLNNFYPREAKVQWKVDNALQSGNSQESVTEQD |
| Light Chain | SKDSTYSLSSTLTLSKADYEKHKVYACEVTHQGL |
| | SSPVTKSFNRGEC (SEQ ID NO: 66) |
| huD39.5.2.3- | QVQLVQSGAEVVKPGASVKLSCKASGYTFTTFWM |
| huG4a_hole_RF_ | HWVRQAPGQGLEWIGNIYPGSGTINYDEKFRSRAT |
| huE15.1_scFvds- | LTVDTSISTAYMEVSRLRSEDTAVYYCTTGWDGEH |
| huG4a_hingeFc_knob_ | WGQGTTLTVSSASTKGPSVFPLAPCSRSTSESTAAL |
| KiHss | GCLVKDYFPEPVTVSWNSGALTSGVHTFPAVLQSS |
| Heavy Chain 1 | GLYSLSSVVTVPSSSLGTKTYTCNVDHKPSNTKVDK |
| | RVESKYGPPCPPCPAPEFLGGPSVFLFPPKPKDTLMIS |
| | RTPEVTCVVVDVSQEDPEVQFNWYVDGVEVHNAKT |
| | KPREEQFNSTYRVVSVLTVLHQDWLNGKEYKCKVSN |
| | KGLPSSIEKTISKAKGQPREPQVYTLPPCQEEMTKNQV |
| | SLSCAVKGFYPSDIAVEWESNGQPENNYKTTPPVLDSD |
| | GSFFLVSRLTVDKSRWQEGNVFSCSVMHEALHNRFTQ |
| | KSLSLSLG (SEQ ID NO: 67) |
| huD39.5.2.3- | EVQLQQSGAEVKKPGASVKISCKASGYTFTGYYMNW |
| huG4a_hole_RF_ | MKQSHGKCLEWIGDLNPDNGDTNYNQKFVGRATLTV |
| huE15.1_scFvds- | DKSISTAYMELSRLRSEDTAVYYCARGGKGGFDYWGQ |
| huG4a_hingeFc_knob_ | GTTLTVSSGGGGSGGGGSGGGGSADVVMTQSPLSLPVT |
| KiHss | LGQPASISCRSSQSLEKSNGNTYLNWYLQRPGQSPQLLIY |
| Heavy Chain 2 | RVSRRYSGVPDRFSGSGSGTDFTLKISRVEAEDVGVYYCL |
| | QVTHVPYTFGCGTRLEIKESKYGPPCPPCPAPEFLGGPSVF |
| | LFPPKPKDTLMISRTPEVTCVVVDVSQEDPEVQFNWYVD |
| | GVEVHNAKTKPREEQFNSTYRVVSVLTVLHQDWLNGKE |
| | YKCKVSNKGLPSSIEKTISKAKGQPREPQVCTLPPSQEEM |
| | TKNQVSLWCLVKGFYPSDIAVEWESNGQPENNYKTTPPV |
| | LDSDGSFFLYSRLTVDKSRWQEGNVFSCSVMHEALHNHYTQK |
| | SLSLSLG (SEQ ID NO: 68) |

### CD47 Embodiment Sequences:

| Name: | Sequence: |
|---|---|
| huD39.5.2.3- | DIQMTQSPSSLSVSVGDRVTITCRASENIHS |
| huG4a_hole_RF_huE24.6_ | NLAWYQQKPGKAPQLLVYGATNLADGVP |
| scFvds-huG4a_hingeFc_knob_ | SRFSGSGSGAQYTLTISSLQPEDFATYYCQH |
| KiHss | FWGTPPYAFGGGTKLEIKRTVAAPSVFIFPPS |
| Light Chain | DEQLKSGTASVVCLLNNFYPREAKVQWKVD |
| | NALQSGNSQESVTEQDSKDSTYSLSSTLTLSK |
| | ADYEKHKVYACEVTHQGLSSPVTKSFNRGEC |
| | (SEQ ID NO: 69) |
| huD39.5.2.3- | QVQLVQSGAEVVKPGASVKLSCKASGYTFTT |
| huG4a_hole_RF_huE24.6_ | FWMHWVRQAPGQGLEWIGNIYPGSGTINYD |
| scFvds-huG4a_hingeFc_knob_ | EKFRSRATLTVDTSISTAYMEVSRLRSEDTAV |
| KiHss | YYCTTGWDGEHWGQGTTLTVSSASTKGPSVF |
| Heavy Chain 1 | PLAPCSRSTSESTAALGCLVKDYFPEPVTVSW |
| | NSGALTSGVHTFPAVLQSSGLYSLSSVVTVPSSS |
| | LGTKTYTCNVDHKPSNTKVDKRVESKYGPPCPP |
| | CPAPEFLGGPSVFLFPPKPKDTLMISRTPEVTCVV |
| | VDVSQEDPEVQFNWYVDGVEVHNAKTKPREEQ |
| | FNSTYRVVSVLTVLHQDWLNGKEYKCKVSNKGL |
| | PSSIEKTISKAKGQPREPQVYTLPPCQEEMTKNQV |
| | SLSCAVKGFYPSDIAVEWESNGQPENNYKTTPPVL |
| | DSDGSFFLVSRLTVDKSRWQEGNVFSCSVMHEAL |
| | HNRFTQKSLSLSLG (SEQ ID NO: 70) |
| huD39.5.2.3- | QIQLQQSGAEVKKPGASVKISCKASGYTFIDYY |
| huG4a_hole_RF_huE24.6_ | INWVKQRPGQCLEWIGWIYPGSGNTKYNEKFK |
| scFvds-huG4a_hingeFc_knob_ | DRGTLTVDTSSSTAYMELSSLRSEDTAVYFCVR |
| KiHss | KGIIYNYGSSDVLAYWGQGTLVTVSSGGGGSGG |
| Heavy Chain 2 | GGSGGGGSADIVMTQSPDSLAVSLGERLTINCKSS |
| | QSLLYSSNQKNYLAWYQQKPGQSPKLLIYWASTR |
| | ESGVPDRFSGSGSGTDFTLTISSVQAEDVAVYYCHQ |
| | YYSYPLTFGCGTKLELKESKYGPPCPPCPAPEFLGGP |
| | SVFLFPPKPKDTLMISRTPEVTCVVVDVSQEDPEVQF |
| | NWYVDGVEVHNAKTKPREEQFNSTYRVVSVLTVLH |
| | QDWLNGKEYKCKVSNKGLPSSIEKTISKAKGQPREPQ |
| | VCTLPPSQEEMTKNQVSLWCLVKGFYPSDIAVEWES |
| | NGQPENNYKTTPPVLDSDGSFFLYSRLTVDKSRWQE |
| | GNVFSCSVMHEALHNHYTQKSLSLSLG |
| | (SEQ ID NO: 71) |

huIgG4 Sequences:

| Name: | Sequence: |
|---|---|
| huG4a_hole | ASTKGPSVFPLAPCSRSTSESTAALGCLVKDYFPEPVTVSWNS |
| | GALTSGVHTFPAVLQSSGLYSLSSVVTVPSSSLGTKTYTCNVD |
| | HKPSNTKVDKRVESKYGPPCPPCPAPEFLGGPSVFLFPPKPKD |
| | TLMISRTPEVTCVVVDVSQEDPEVQFNWYVDGVEVHNAKTK |
| | PREEQFNSTYRVVSVLTVLHQDWLNGKEYKCKVSNKGLPSSI |
| | EKTISKAKGQPREPQVYTLPPSQEEMTKNQVSLSCAVKGFYPS |
| | DIAVEWESNGQPENNYKTTPPVLDSDGSFFLVSRLTVDKSRW |
| | QEGNVFSCSVMHEALHNHYTQKSLSLSLG (SEQ ID NO: 76) |
| huG4a_hole_SC_HR_YF | ASTKGPSVFPLAPCSRSTSESTAALGCLVKDYFPEPVTVSWNS |
| | GALTSGVHTFPAVLQSSGLYSLSSVVTVPSSSLGTKTYTCNVD |
| | HKPSNTKVDKRVESKYGPPCPPCPAPEFLGGPSVFLFPPKPKD |
| | TLMISRTPEVTCVVVDVSQEDPEVQFNWYVDGVEVHNAKTK |
| | PREEQFNSTYRVVSVLTVLHQDWLNGKEYKCKVSNKGLPSSI |
| | EKTISKAKGQPREPQVYTLPPCQEEMTKNQVSLSCAVKGFYPS |
| | DIAVEWESNGQPENNYKTTPPVLDSDGSFFLVSRLTVDKSRW |
| | QEGNVFSCSVMHEALHNRFTQKSLSLSLG (SEQ ID NO: 77) |
| huG4a_hole_YC_HR_YF | ASTKGPSVFPLAPCSRSTSESTAALGCLVKDYFPEPVTVSWNS |
| | GALTSGVHTFPAVLQSSGLYSLSSVVTVPSSSLGTKTYTCNVD |
| | HKPSNTKVDKRVESKYGPPCPPCPAPEFLGGPSVFLFPPKPKD |
| | TLMISRTPEVTCVVVDVSQEDPEVQFNWYVDGVEVHNAKTK |
| | PREEQFNSTYRVVSVLTVLHQDWLNGKEYKCKVSNKGLPSSI |
| | EKTISKAKGQPREPQVCTLPPSQEEMTKNQVSLSCAVKGFYPS |
| | DIAVEWESNGQPENNYKTTPPVLDSDGSFFLVSRLTVDKSRW |
| | QEGNVFSCSVMHEALHNRFTQKSLSLSLG (SEQ ID NO: 78) |
| huG4a_hole_SC | ASTKGPSVFPLAPCSRSTSESTAALGCLVKDYFPEPVTVSWNS |
| | GALTSGVHTFPAVLQSSGLYSLSSVVTVPSSSLGTKTYTCNVD |
| | HKPSNTKVDKRVESKYGPPCPPCPAPEFLGGPSVFLFPPKPKD |
| | TLMISRTPEVTCVVVDVSQEDPEVQFNWYVDGVEVHNAKTK |
| | PREEQFNSTYRVVSVLTVLHQDWLNGKEYKCKVSNKGLPSSI |
| | EKTISKAKGQPREPQVYTLPPCQEEMTKNQVSLSCAVKGFYPS |
| | DIAVEWESNGQPENNYKTTPPVLDSDGSFFLVSRLTVDKSRW |
| | QEGNVFSCSVMHEALHNHYTQKSLSLSLG (SEQ ID NO: 79) |
| huG4a_hole_YC | ASTKGPSVFPLAPCSRSTSESTAALGCLVKDYFPEPVTVSWNS |
| | GALTSGVHTFPAVLQSSGLYSLSSVVTVPSSSLGTKTYTCNVD |
| | HKPSNTKVDKRVESKYGPPCPPCPAPEFLGGPSVFLFPPKPKD |
| | TLMISRTPEVTCVVVDVSQEDPEVQFNWYVDGVEVHNAKTK |
| | PREEQFNSTYRVVSVLTVLHQDWLNGKEYKCKVSNKGLPSSI |
| | EKTISKAKGQPREPQVCTLPPSQEEMTKNQVSLSCAVKGFYPS |
| | DIAVEWESNGQPENNYKTTPPVLDSDGSFFLVSRLTVDKSRW |
| | QEGNVFSCSVMHEALHNHYTQKSLSLSLG (SEQ ID NO: 80) |
| huG4a_hingeFc_knob | ESKYGPPCPPCPAPEFLGGPSVFLFPPKPKDTLMISRTPEVTCV |
| | VVDVSQEDPEVQFNWYVDGVEVHNAKTKPREEQFNSTYRVV |
| | SVLTVLHQDWLNGKEYKCKVSNKGLPSSIEKTISKAKGQPRE |
| | PQVYTLPPSQEEMTKNQVSLWCLVKGFYPSDIAVEWESNGQP |
| | ENNYKTTPPVLDSDGSFFLYSRLTVDKSRWQEGNVFSCSVMH |
| | EALHNHYTQKSLSLSLG (SEQ ID NO: 81) |
| huG4a_hingeFc_knob_YC | ESKYGPPCPPCPAPEFLGGPSVFLFPPKPKDTLMISRTPEVTCV |
| | VVDVSQEDPEVQFNWYVDGVEVHNAKTKPREEQFNSTYRVV |
| | SVLTVLHQDWLNGKEYKCKVSNKGLPSSIEKTISKAKGQPRE |
| | PQVCTLPPSQEEMTKNQVSLWCLVKGFYPSDIAVEWESNGQP |
| | ENNYKTTPPVLDSDGSFFLYSRLTVDKSRWQEGNVFSCSVMH |
| | EALHNHYTQKSLSLSLG (SEQ ID NO: 82) |
| huG4a_hingeFc_knob_SC | ESKYGPPCPPCPAPEFLGGPSVFLFPPKPKDTLMISRTPEVTCV |
| | VVDVSQEDPEVQFNWYVDGVEVHNAKTKPREEQFNSTYRVV |
| | SVLTVLHQDWLNGKEYKCKVSNKGLPSSIEKTISKAKGQPRE |
| | PQVYTLPPCQEEMTKNQVSLWCLVKGFYPSDIAVEWESNGQP |
| | ENNYKTTPPVLDSDGSFFLYSRLTVDKSRWQEGNVFSCSVMH |
| | EALHNHYTQKSLSLSLG (SEQ ID NO: 83) |
| huG4a_hingeFc_knob_ | ESKYGPPCPPCPAPEFLGGPSVFLFPPKPKDTLMISRTPEVTCV |
| YC_HR_YF | VVDVSQEDPEVQFNWYVDGVEVHNAKTKPREEQFNSTYRVV |
| | SVLTVLHQDWLNGKEYKCKVSNKGLPSSIEKTISKAKGQPRE |
| | PQVCTLPPSQEEMTKNQVSLWCLVKGFYPSDIAVEWESNGQP |
| | ENNYKTTPPVLDSDGSFFLYSRLTVDKSRWQEGNVFSCSVMH |
| | EALHNRFTQKSLSLSLG (SEQ ID NO: 84) |
| huG4a_hingeFc_knob_ | ESKYGPPCPPCPAPEFLGGPSVFLFPPKPKDTLMISRTPEVTCV |
| SC_HR_YF | VVDVSQEDPEVQFNWYVDGVEVHNAKTKPREEQFNSTYRVV |
| | SVLTVLHQDWLNGKEYKCKVSNKGLPSSIEKTISKAKGQPRE |
| | PQVYTLPPCQEEMTKNQVSLWCLVKGFYPSDIAVEWESNGQP |
| | ENNYKTTPPVLDSDGSFFLYSRLTVDKSRWQEGNVFSCSVMH |
| | EALHNRFTQKSLSLSLG (SEQ ID NO: 85) |

### Additional Linkers:

| Linker Name: | Sequence: |
|---|---|
| 5 AA | GGGGS (SEQ ID NO: 36) |
| 10 AA | GGGGSGGGGS (SEQ ID NO: 37) |
| 12 AA | GGSGGSGGSGGS (SEQ ID NO: 120) |
| 20 AA | GGGGSGGGGSGGGGSGGGGS (SEQ ID NO: 121) |
| 25 AA | GGGGSGGGGSGGGGSGGGGSGGGGS (SEQ ID NO: 122) |
| 30 AA | GGGGSGGGGSGGGGSGGGGSGGGGSGGGGS |
| | (SEQ ID NO: 123) |
| 35 AA | GGGGSGGGGSGGGGSGGGGSGGGGSGGGGSGGGGS |
| | (SEQ ID NO: 124) |
| MMP14_uPA | GSSGRIGFLRTAGSLGGSGRSANAILEGS (SEQ ID NO: 125) |
| uPA_MMP14 | GSLGGSGRSANAILEGSSGRIGFLRTAGS (SEQ ID NO: 126) |
| MMP14_uPA_long | GGGGSSGRIGFLRTAGGGGSLGGSGRSANAILEGGGGS |
| | (SEQ ID NO: 127) |
| uPA_MMP14_long | GGGGSLGGSGRSANAILEGGGGSSGRIGFLRTAGGGGS |
| | (SEQ ID NO: 128) |

### IL-15 Sequences:

| Name: | Sequence: |
|---|---|
| IL15 | NWVNVISDLKKIEDLIQSMHIDATLYTESDVHPSCKVTAMKCFLLELQVISLESG |
| | DASIHDTVENLIILANNSLSSNGNVTESGCKECEELEEKNIKEFLQSFVHIVQMFIN |
| | TS (SEQ ID NO: 86) |
| IL15Ra | ITCPPPMSVEHADIWVKSYSLYSRERYICNSGFKRKAGTSSLTECVLNKATNVAH |
| | WTTPSLKCIRDPALVHQRPAPPSTVTTAGVTPQPESLSPSGKEPAASSPSSNNTAA |
| | TTAAIVPGSQLMPSKSPSTGTTEISSHESSHGTPSQTTAKNWELTASASHQPPGVY |
| | PQGHSDTT (SEQ ID NO: 87) |
| IL15RaSu | ITCPPPMSVEHADIWVKSYSLYSRERYICNSGFKRKAGTSSLTECVLNKATNVAH |
| | WTTPSLKCIR (SEQ ID NO: 88) |
| IL15Rb- | AVNGTSQFTCFYNSRANISCVWSQDGALQDTSCQVHAWPDRRRWNQTCELLPV |
| ECD | SQASWACNLILGAPDSQKLTTVDIVTLRVLCREGVRWRVMAIQDFKPFENLRLM |
| | APISLQVVHVETHRCNISWEISQASHYFERHLEFEARTLSPGHTWEEAPLLTLKQ |
| | KQEWICLETLTPDTQYEFQVRVKPLQGEFTTWSPWSQPLAFRTKPAALGKDT |
| | (SEQ ID NO: 89) |
| IL15Rb-D1 | AVNGTSQFTCFYNSRANISCVWSQDGALQDTSCQVHAWPDRRRWNQTCELLPV |
| | SQASWACNLILGAPDSQKLTTVDIVTLRVLCREGVRWRVMAIQDFKPFEN (SEQ |
| | ID NO: 90) |

### Constant Region Sequences:

| Name: | Sequence: |
|---|---|
| CK (light chain | RTVAAPSVFIFPPSDEQLKSGTASVVCLLNNFYPREAKVQWKVDNALQ |
| kappa constant | SGNSQESVTEQDSKDSTYSLSSTLTLSKADYEKHKVYACEVTHQGLSS |
| region) | PVTKSFNRGEC (SEQ ID NO: 91) |
| IgG1 | ASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDK |
| | KVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSF |
| | FLYSKLTVDKSRWQQGNVFSCSVMHEALHNHYTQKSLSLSPG (SEQ |
| | ID NO: 92) |
| IgG1AAA | ASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSRD |
| | ELTKNQVSLTCLVKGFYPSDIAVEWESNGQPENNYKTTPPVLDSDGSF |
| | FLYSKLTVDKSRWQQGNVFSCSVMHEALHNHYTQKSLSLSPG (SEQ |
| | ID NO: 93) |
| IgG1AAA_knob | ASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSRD |
| | ELTKNQVSLWCLVKGFYPSDIAVEWESNGQPENNYKTTPPVLDSDGSF |
| | FLYSKLTVDKSRWQQGNVFSCSVMHEALHNHYTQKSLSLSPG (SEQ |
| | ID NO: 94) |
| IgG1AAA_hole | ASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPSRD |
| | ELTKNQVSLSCLAKGFYPSDIAVEWESNGQPENNYKTTPPVLDSDGSF |
| | FLVSKLTVDKSRWQQGNVFSCSVMHEALHNHYTQKSLSLSPG (SEQ |
| | ID NO: 95) |
| IgG1AAA_knob_YC | ASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVCTLPPSRD |
| | ELTKNQVSLWCLVKGFYPSDIAVEWESNGQPENNYKTTPPVLDSDGSF |
| | FLYSKLTVDKSRWQQGNVFSCSVMHEALHNHYTQKSLSLSPG (SEQ |
| | ID NO: 96) |
| IgG1AAA_hole_SC | ASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPCRD |
| | ELTKNQVSLSCLAKGFYPSDIAVEWESNGQPENNYKTTPPVLDSDGSF |
| | FLVSKLTVDKSRWQQGNVFSCSVMHEALHNHYTQKSLSLSPG (SEQ |
| | ID NO: 97) |
| IgG1AAA_knob_SC | ASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPCRD |
| | ELTKNQVSLWCLVKGFYPSDIAVEWESNGQPENNYKTTPPVLDSDGSF |
| | FLYSKLTVDKSRWQQGNVFSCSVMHEALHNHYTQKSLSLSPG (SEQ |
| | ID NO: 98) |
| IgG1AAA_hole_YC | ASTKGPSVFPLAPSSKSTSGGTAALGCLVKDYFPEPVTVSWNSGALTS |
| | GVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVCTLPPSRD |
| | ELTKNQVSLSCLAKGFYPSDIAVEWESNGQPENNYKTTPPVLDSDGSF |
| | FLVSKLTVDKSRWQQGNVFSCSVMHEALHNHYTQKSLSLSPG (SEQ |
| | ID NO: 99) |
| IgGIAAA_knob_YC_ | ASTKGPSVFPLAPSSKSTSGGTAALGCLVKDYFPEPVTVSWNSGALTS |
| RF | GVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVCTLPPSRD |
| | ELTKNQVSLWCLVKGFYPSDIAVEWESNGQPENNYKTTPPVLDSDGSF |
| | FLYSKLTVDKSRWQQGNVFSCSVMHEALHNRFTQKSLSLSPG (SEQ ID |
| | NO: 100) |
| IgG1AAA_hole_SC_ | ASTKGPSVFPLAPSSKSTSGGTAALGCLVKDYFPEPVTVSWNSGALTS |
| RF | GVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPCRD |
| | ELTKNQVSLSCLAKGFYPSDIAVEWESNGQPENNYKTTPPVLDSDGSF |
| | FLVSKLTVDKSRWQQGNVFSCSVMHEALHNRFTQKSLSLSPG (SEQ ID |
| | NO: 101) |
| IgG1AAA_knob_SC_ | ASTKGPSVFPLAPSSKSTSGGTAALGCLVKDYFPEPVTVSWNSGALTS |
| RF | GVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVYTLPPCRD |
| | ELTKNQVSLWCLVKGFYPSDIAVEWESNGQPENNYKTTPPVLDSDGSF |
| | FLYSKLTVDKSRWQQGNVFSCSVMHEALHNRFTQKSLSLSPG (SEQ ID |
| | NO: 102) |
| IgG1AAA_hole_YC_ | ASTKGPSVFPLAPSSKSTSGGTAALGCLVKDYFPEPVTVSWNSGALTS |
| RF | GVHTFPAVLQSSGLYSLSSVVTVPSSSLGTQTYICNVNHKPSNTKVDK |
| | KVEPKSCDKTHTCPPCPAPEAAGAPSVFLFPPKPKDTLMISRTPEVTCV |
| | VVDVSHEDPEVKFNWYVDGVEVHNAKTKPREEQYNSTYRVVSVLTV |
| | LHQDWLNGKEYKCKVSNKALPAPIEKTISKAKGQPREPQVCTLPPSRD |
| | ELTKNQVSLSCLAKGFYPSDIAVEWESNGQPENNYKTTPPVLDSDGSF |
| | FLVSKLTVDKSRWQQGNVFSCSVMHEALHNRFTQKSLSLSPG (SEQ ID |
| | NO: 103) |

### IL-15 Full Length Sequences:

| Name: | Sequence: |
|---|---|
| D39.5.2.3-G1AAA- | DIQMTQSPSSLSVSVGDRVTITCRASENIHSNL |
| IL15RaSu-IL15-T2A | AWYQQKPGKAPQLLVYGATNLADGVPSRFS |
| Light Chain | GSGSGAQYTLTISSLQPEDFATYYCQHFWGT |
| | PPYAFGGGTKLEIKRTVAAPSVFIFPPSDEQLK |
| | SGTASVVCLLNNFYPREAKVQWKVDNALQS |
| | GNSQESVTEQDSKDSTYSLSSTLTLSKADYEK |
| | HKVYACEVTHQGLSSPVTKSFNRGEC |
| | (SEQ ID NO: 104) |
| D39.5.2.3-G1AAA- | QVQLVQSGAEVVKPGASVKLSCKASGYTFTTF |
| IL15RaSu-IL15-T2A | WMHWVRQAPGQGLEWIGNIYPGSGTINYDEK |
| Heavy Chain | FRSRATLTVDTSISTAYMEVSRLRSEDTAVYYC |
| | TTGWDGEHWGQGTTLTVSSASTKGPSVFPLAP |
| | SSKSTSGGTAALGCLVKDYFPEPVTVSWNSG |
| | ALTSGVHTFPAVLQSSGLYSLSSVVTVPSSSLGT |
| | QTYICNVNHKPSNTKVDKKVEPKSCDKTHTCPP |
| | CPAPEAAGAPSVFLFPPKPKDTLMISRTPEVTCV |
| | VVDVSHEDPEVKFNWYVDGVEVHNAKTKPREE |
| | QYNSTYRVVSVLTVLHQDWLNGKEYKCKVSNK |
| | ALPAPIEKTISKAKGQPREPQVYTLPPSRDELTKN |
| | QVSLTCLVKGFYPSDIAVEWESNGQPENNYKTTP |
| | PVLDSDGSFFLYSKLTVDKSRWQQGNVFSCSVM |
| | HEALHNHYTQKSLSLSPGGGGGSGGGGSITCPPP |
| | MSVEHADIWVKSYSLYSRERYICNSGFKRKAGTS |
| | SLTECVLNKATNVAHWTTPSLKCIRGGGGSGGGGS |
| | GGGGSGGGGSGGGGSNWVNVISDLKKIEDLIQSMHI |
| | DATLYTESDVHPSCKVTAMKCFLLELQVISLESGDA |
| | SIHDTVENLIILANNSLSSNGNVTESGCKECEELEEK |
| | NIKEFLQSFVHIVQMFINTS (SEQ ID NO: 105) |
| D39.5.2.3-G1AAA-IL15- | DIQMTQSPSSLSVSVGDRVTITCRASENIHSNLAWY |
| IL15RaSu-T2B | QQKPGKAPQLLVYGATNLADGVPSRFSGSGSGAQ |
| Light Chain | YTLTISSLQPEDFATYYCQHFWGTPPYAFGGGTKL |
| | EIKRTVAAPSVFIFPPSDEQLKSGTASVVCLLNNFY |
| | PREAKVQWKVDNALQSGNSQESVTEQDSKDSTYS |
| | LSSTLTLSKADYEKHKVYACEVTHQGLSSPVTKSF |
| | NRGEC (SEQ ID NO: 106) |
| D39.5.2.3-G1AAA-IL15- | QVQLVQSGAEVVKPGASVKLSCKASGYTFTTF |
| IL15RaSu-T2B | WMHWVRQAPGQGLEWIGNIYPGSGTINYDEKF |
| Heavy Chain | RSRATLTVDTSISTAYMEVSRLRSEDTAVYYCTT |
| | GWDGEHWGQGTTLTVSSASTKGPSVFPLAPSSK |
| | STSGGTAALGCLVKDYFPEPVTVSWNSGALTSG |
| | VHTFPAVLQSSGLYSLSSVVTVPSSSLGTQTYICN |
| | VNHKPSNTKVDKKVEPKSCDKTHTCPPCPAPEAA |
| | GAPSVFLFPPKPKDTLMISRTPEVTCVVVDVSHED |
| | PEVKFNWYVDGVEVHNAKTKPREEQYNSTYRVV |
| | SVLTVLHQDWLNGKEYKCKVSNKALPAPIEKTISK |
| | AKGQPREPQVYTLPPSRDELTKNQVSLTCLVKGFY |
| | PSDIAVEWESNGQPENNYKTTPPVLDSDGSFFLYSK |
| | LTVDKSRWQQGNVFSCSVMHEALHNHYTQKSLSLS |
| | PGGGGGSGGGGSNWVNVISDLKKIEDLIQSMHIDAT |
| | LYTESDVHPSCKVTAMKCFLLELQVISLESGDASIHD |
| | TVENLIILANNSLSSNGNVTESGCKECEELEEKNIKEF |
| | LQSFVHIVQMFINTSGGGGSGGGGSGGGGSGGGGS |
| | GGGGSITCPPPMSVEHADIWVKSYSLYSRERYICNSG |
| | FKRKAGTSSLTECVLNKATNVAHWTTPSLKCIR |
| | (SEQ ID NO: 107) |
| D39.5.2.3-G1AAA.KiH- | DIQMTQSPSSLSVSVGDRVTITCRASENIHSNLAW |
| IL15 + IL15RaSu-T3 | YQQKPGKAPQLLVYGATNLADGVPSRFSGSGSGA |
| Light Chain | QYTLTISSLQPEDFATYYCQHFWGTPPYAFGGGTK |
| | LEIKRTVAAPSVFIFPPSDEQLKSGTASVVCLLNNFY |
| | PREAKVQWKVDNALQSGNSQESVTEQDSKDSTYSL |
| | SSTLTLSKADYEKHKVYACEVTHQGLSSPVTKSFN |
| | RGEC (SEQ ID NO: 108) |
| D39.5.2.3-G1AAA.KiH- | QVQLVQSGAEVVKPGASVKLSCKASGY |
| IL15 + IL15RaSu-T3 | TFTTFWMHWVRQAPGQGLEWIGNIYPG |
| Heavy Chain 1 | SGTINYDEKFRSRATLTVDTSISTAYMEV |
| | SRLRSEDTAVYYCTTGWDGEHWGQGTT |
| | LTVSSASTKGPSVFPLAPSSKSTSGGTAAL |
| | GCLVKDYFPEPVTVSWNSGALTSGVHTFP |
| | AVLQSSGLYSLSSVVTVPSSSLGTQTYICN |
| | VNHKPSNTKVDKKVEPKSCDKTHTCPPCP |
| | APEAAGAPSVFLFPPKPKDTLMISRTPEVTC |
| | VVVDVSHEDPEVKFNWYVDGVEVHNAKT |
| | KPREEQYNSTYRVVSVLTVLHQDWLNGKE |
| | YKCKVSNKALPAPIEKTISKAKGQPREPQVY |
| | TLPPSRDELTKNQVSLSCLAKGFYPSDIAVE |
| | WESNGQPENNYKTTPPVLDSDGSFFLVSKLT |
| | VDKSRWQQGNVFSCSVMHEALHNHYTQKS |
| | LSLSPGGGGGSGGGGSGGGGSGGGGSGGGG |
| | SNWVNVISDLKKIEDLIQSMHIDATLYTESDV |
| | HPSCKVTAMKCFLLELQVISLESGDASIHDTV |
| | ENLIILANNSLSSNGNVTESGCKECEELEEKNI |
| | KEFLQSFVHIVQMFINTS (SEQ ID NO: 109) |
| D39.5.2.3-G1AAA.KiH- | QVQLVQSGAEVVKPGASVKLSCKASGYTFTT |
| IL15 + IL15RaSu-T3 | FWMHWVRQAPGQGLEWIGNIYPGSGTINYDE |
| Heavy Chain 2 | KFRSRATLTVDTSISTAYMEVSRLRSEDTAVY |
| | YCTTGWDGEHWGQGTTLTVSSASTKGPSVFPL |
| | APSSKSTSGGTAALGCLVKDYFPEPVTVSWNSG |
| | ALTSGVHTFPAVLQSSGLYSLSSVVTVPSSSLGT |
| | QTYICNVNHKPSNTKVDKKVEPKSCDKTHTCPP |
| | CPAPEAAGAPSVFLFPPKPKDTLMISRTPEVTCV |
| | VVDVSHEDPEVKFNWYVDGVEVHNAKTKPREE |
| | QYNSTYRVVSVLTVLHQDWLNGKEYKCKVSNK |
| | ALPAPIEKTISKAKGQPREPQVYTLPPSRDELTKN |
| | QVSLWCLVKGFYPSDIAVEWESNGQPENNYKTT |
| | PPVLDSDGSFFLYSKLTVDKSRWQQGNVFSCSVM |
| | HEALHNHYTQKSLSLSPGGGGGSGGGGSGGGGS |
| | GGGGSGGGGSITCPPPMSVEHADIWVKSYSLYSR |
| | ERYICNSGFKRKAGTSSLTECVLNKATNVAHWTT |
| | PSLKCIR (SEQ ID NO: 110) |
| D39.5.2.3-G1AAA- | DIQMTQSPSSLSVSVGDRVTITCRASENIHSNLAW |
| IL15RaSu-IL15-T2A-mono | YQQKPGKAPQLLVYGATNLADGVPSRFSGSGSG |
| Light Chain | AQYTLTISSLQPEDFATYYCQHFWGTPPYAFGGG |
| | TKLEIKRTVAAPSVFIFPPSDEQLKSGTASVVCLLN |
| | NFYPREAKVQWKVDNALQSGNSQESVTEQDSKD |
| | STYSLSSTLTLSKADYEKHKVYACEVTHQGLSSP |
| | VTKSFNRGEC (SEQ ID NO: 111) |
| D39.5.2.3-G1AAA- | QVQLVQSGAEVVKPGASVKLSCKASGYT |
| IL15RaSu-IL15-T2A-mono | FTTFWMHWVRQAPGQGLEWIGNIYPGSG |
| Heavy Chain 1 | TINYDEKFRSRATLTVDTSISTAYMEVSRL |
| | RSEDTAVYYCTTGWDGEHWGQGTTLTVS |
| | SASTKGPSVFPLAPSSKSTSGGTAALGCLV |
| | KDYFPEPVTVSWNSGALTSGVHTFPAVLQ |
| | SSGLYSLSSVVTVPSSSLGTQTYICNVNHKP |
| | SNTKVDKKVEPKSCDKTHTCPPCPAPEAAG |
| | APSVFLFPPKPKDTLMISRTPEVTCVVVDVS |
| | HEDPEVKFNWYVDGVEVHNAKTKPREEQY |
| | NSTYRVVSVLTVLHQDWLNGKEYKCKVSN |
| | KALPAPIEKTISKAKGQPREPQVYTLPPSRDE |
| | LTKNQVSLWCLVKGFYPSDIAVEWESNGQP |
| | ENNYKTTPPVLDSDGSFFLYSKLTVDKSRWQ |
| | QGNVFSCSVMHEALHNHYTQKSLSLSPGGGG |
| | GSGGGGSITCPPPMSVEHADIWVKSYSLYSRE |
| | RYICNSGFKRKAGTSSLTECVLNKATNVAHW |
| | TTPSLKCIRGGGGSGGGGSGGGGSGGGGSGG |
| | GGSNWVNVISDLKKIEDLIQSMHIDATLYTESD |
| | VHPSCKVTAMKCFLLELQVISLESGDASIHDTV |
| | ENLIILANNSLSSNGNVTESGCKECEELEEKNIK |
| | EFLQSFVHIVQMFINTS (SEQ ID NO: 112) |
| D39.5.2.3-G1AAA- | QVQLVQSGAEVVKPGASVKLSCKASGYTFTTF |
| IL15RaSu-IL15-T2A-mono | WMHWVRQAPGQGLEWIGNIYPGSGTINYDEKF |
| Heavy Chain 2 | RSRATLTVDTSISTAYMEVSRLRSEDTAVYYCT |
| | TGWDGEHWGQGTTLTVSSASTKGPSVFPLAPSS |
| | KSTSGGTAALGCLVKDYFPEPVTVSWNSGALTS |
| | GVHTFPAVLQSSGLYSLSSVVTVPSSSLGTQTYIC |
| | NVNHKPSNTKVDKKVEPKSCDKTHTCPPCPAPEA |
| | AGAPSVFLFPPKPKDTLMISRTPEVTCVVVDVSHE |
| | DPEVKFNWYVDGVEVHNAKTKPREEQYNSTYRV |
| | VSVLTVLHQDWLNGKEYKCKVSNKALPAPIEKTIS |
| | KAKGQPREPQVYTLPPSRDELTKNQVSLSCLAKGF |
| | YPSDIAVEWESNGQPENNYKTTPPVLDSDGSFFLV |
| | SKLTVDKSRWQQGNVFSCSVMHEALHNHYTQKSL |
| | SLSPG (SEQ ID NO: 113) |
| D39.5.2.3-G1AAA-IL15- | DIQMTQSPSSLSVSVGDRVTITCRASENIHS |
| IL15RaSu-T2B-mono | NLAWYQQKPGKAPQLLVYGATNLADGVP |
| Light Chain | SRFSGSGSGAQYTLTISSLQPEDFATYYCQH |
| | FWGTPPYAFGGGTKLEIKRTVAAPSVFIFPP |
| | SDEQLKSGTASVVCLLNNFYPREAKVQWK |
| | VDNALQSGNSQESVTEQDSKDSTYSLSSTLT |
| | LSKADYEKHKVYACEVTHQGLSSPVTKSFN |
| | RGEC (SEQ ID NO: 114) |
| D39.5.2.3-G1AAA-IL15- | QVQLVQSGAEVVKPGASVKLSCKASGYTF |
| IL15RaSu-T2B-mono | TTFWMHWVRQAPGQGLEWIGNIYPGSGTI |
| Heavy Chain 1 | NYDEKFRSRATLTVDTSISTAYMEVSRLRS |
| | EDTAVYYCTTGWDGEHWGQGTTLTVSSA |
| | STKGPSVFPLAPSSKSTSGGTAALGCLVKD |
| | YFPEPVTVSWNSGALTSGVHTFPAVLQSSG |
| | LYSLSSVVTVPSSSLGTQTYICNVNHKPSNT |
| | KVDKKVEPKSCDKTHTCPPCPAPEAAGAPS |
| | VFLFPPKPKDTLMISRTPEVTCVVVDVSHED |
| | PEVKFNWYVDGVEVHNAKTKPREEQYNST |
| | YRVVSVLTVLHQDWLNGKEYKCKVSNKAL |
| | PAPIEKTISKAKGQPREPQVYTLPPSRDELTK |
| | NQVSLWCLVKGFYPSDIAVEWESNGQPENN |
| | YKTTPPVLDSDGSFFLYSKLTVDKSRWQQGN |
| | VFSCSVMHEALHNHYTQKSLSLSPGGGGGSG |
| | GGGSNWVNVISDLKKIEDLIQSMHIDATLYTE |
| | SDVHPSCKVTAMKCFLLELQVISLESGDASIHD |
| | TVENLIILANNSLSSNGNVTESGCKECEELEEK |
| | NIKEFLQSFVHIVQMFINTSGGGGSGGGGSGGG |
| | GSGGGGSGGGGSGGGGSGGGGSITCPPPMSVEH |
| | ADIWVKSYSLYSRERYICNSGFKRKAGTSSLTEC |
| | VLNKATNVAHWTTPSLKCIR (SEQ ID NO: 115) |
| D39.5.2.3-G1AAA-IL15- | QVQLVQSGAEVVKPGASVKLSCKASGYTF |
| IL15RaSu-T2B-mono | TTFWMHWVRQAPGQGLEWIGNIYPGSGTI |
| Heavy Chain 2 | NYDEKFRSRATLTVDTSISTAYMEVSRLRS |
| | EDTAVYYCTTGWDGEHWGQGTTLTVSSAS |
| | TKGPSVFPLAPSSKSTSGGTAALGCLVKDYF |
| | PEPVTVSWNSGALTSGVHTFPAVLQSSGLYS |
| | LSSVVTVPSSSLGTQTYICNVNHKPSNTKVD |
| | KKVEPKSCDKTHTCPPCPAPEAAGAPSVFLFP |
| | PKPKDTLMISRTPEVTCVVVDVSHEDPEVKEN |
| | WYVDGVEVHNAKTKPREEQYNSTYRVVSVL |
| | TVLHQDWLNGKEYKCKVSNKALPAPIEKTISK |
| | AKGQPREPQVYTLPPSRDELTKNQVSLSCLAK |
| | GFYPSDIAVEWESNGQPENNYKTTPPVLDSDG |
| | SFFLVSKLTVDKSRWQQGNVFSCSVMHEALH |
| | NHYTQKSLSLSPG (SEQ ID NO: 116) |
| D39.5.2.3-G1AAA- | DIQMTQSPSSLSVSVGDRVTITCRASENIHS |
| IL15RaSu-IL15-T2A-masked | NLAWYQQKPGKAPQLLVYGATNLADGVP |
| Light Chain | SRFSGSGSGAQYTLTISSLQPEDFATYYCQH |
| | FWGTPPYAFGGGTKLEIKRTVAAPSVFIFPP |
| | SDEQLKSGTASVVCLLNNFYPREAKVQWK |
| | VDNALQSGNSQESVTEQDSKDSTYSLSSTL |
| | TLSKADYEKHKVYACEVTHQGLSSPVTKSF |
| | NRGEC (SEQ ID NO: 117) |
| D39.5.2.3-G1AAA- | QVQLVQSGAEVVKPGASVKLSCKAS |
| IL15RaSu-IL15-T2A-masked | GYTFTTFWMHWVRQAPGQGLEWIGN |
| Heavy Chain 1 | IYPGSGTINYDEKFRSRATLTVDTSIST |
| | AYMEVSRLRSEDTAVYYCTTGWDGE |
| | HWGQGTTLTVSSASTKGPSVFPLAPSS |
| | KSTSGGTAALGCLVKDYFPEPVTVSW |
| | NSGALTSGVHTFPAVLQSSGLYSLSSV |
| | VTVPSSSLGTQTYICNVNHKPSNTKVD |
| | KKVEPKSCDKTHTCPPCPAPEAAGAPS |
| | VFLFPPKPKDTLMISRTPEVTCVVVDVS |
| | HEDPEVKFNWYVDGVEVHNAKTKPRE |
| | EQYNSTYRVVSVLTVLHQDWLNGKEY |
| | KCKVSNKALPAPIEKTISKAKGQPREPQ |
| | VYTLPPSRDELTKNQVSLWCLVKGFYPS |
| | DIAVEWESNGQPENNYKTTPPVLDSDGS |
| | FFLYSKLTVDKSRWQQGNVFSCSVMHE |
| | ALHNHYTQKSLSLSPGGGGGSGGGGSIT |
| | CPPPMSVEHADIWVKSYSLYSRERYICNS |
| | GFKRKAGTSSLTECVLNKATNVAHWTTP |
| | SLKCIRGGGGSGGGGSGGGGSGGGGSGG |
| | GGSNWVNVISDLKKIEDLIQSMHIDATLY |
| | TESDVHPSCKVTAMKCFLLELQVISLESGD |
| | ASIHDTVENLIILANNSLSSNGNVTESGCKE |
| | CEELEEKNIKEFLQSFVHIVQMFINTS |
| | (SEQ ID NO: 118) |
| D39.5.2.3-G1AAA- | QVQLVQSGAEVVKPGASVKLSCKASGYTF |
| IL15RaSu-IL15-T2A-masked | TTFWMHWVRQAPGQGLEWIGNIYPGSGTIN |
| Heavy Chain 2 | YDEKFRSRATLTVDTSISTAYMEVSRLRSED |
| | TAVYYCTTGWDGEHWGQGTTLTVSSASTK |
| | GPSVFPLAPSSKSTSGGTAALGCLVKDYFPEP |
| | VTVSWNSGALTSGVHTFPAVLQSSGLYSLSSV |
| | VTVPSSSLGTQTYICNVNHKPSNTKVDKKVE |
| | PKSCDKTHTCPPCPAPEAAGAPSVFLFPPKPK |
| | DTLMISRTPEVTCVVVDVSHEDPEVKFNWYV |
| | DGVEVHNAKTKPREEQYNSTYRVVSVLTVLH |
| | QDWLNGKEYKCKVSNKALPAPIEKTISKAKG |
| | QPREPQVYTLPPSRDELTKNQVSLSCLAKGFY |
| | PSDIAVEWESNGQPENNYKTTPPVLDSDGSFF |
| | LVSKLTVDKSRWQQGNVFSCSVMHEALHNH |
| | YTQKSLSLSPGGSSGRIGFLRTAGSLGGSGRSA |
| | NAILEGSAVNGTSQFTCFYNSRANISCVWSQD |
| | GALQDTSCQVHAWPDRRRWNQTCELLPVSQA |
| | SWACNLILGAPDSQKLTTVDIVTLRVLCREGVR |
| | WRVMAIQDFKPFENLRLMAPISLQVVHVETHR |
| | CNISWEISQASHYFERHLEFEARTLSPGHTWEEA |
| | PLLTLKQKQEWICLETLTPDTQYEFQVRVKPLQG |
| | EFTTWSPWSQPLAFRTKPAALGKDT |
| | (SEQ ID NO: 119) |

### Preparation of Binding Compounds

The multispecific binding compounds of the present invention can be prepared by methods known in the art. For example, binding compounds and antigen-binding fragments thereof can also be produced by recombinant DNA technology, by expression of the encoding nucleic acid in a suitable eukaryotic or prokaryotic host, including, for example, mammalian cells (e.g., CHO cells), E. coli or yeast.

### Pharmaceutical Compositions, Uses and Methods of Treatment

It is another aspect of the present invention to provide pharmaceutical compositions comprising one or more multispecific binding compounds of the present invention in admixture with a suitable pharmaceutically acceptable carrier. Pharmaceutically acceptable carriers as used herein are exemplified, but not limited to, adjuvants, solid carriers, water, buffers, or other carriers used in the art to hold therapeutic components, or combinations thereof.

In one embodiment, a pharmaceutical composition comprises a multispecific binding compound that binds to PD-L1 and 4-1BB. In one embodiment, a pharmaceutical composition comprises a multispecific binding compound that binds to PD-L1 and CD47. In one embodiment, a pharmaceutical composition comprises a multispecific binding compound that binds to PD-L1 and comprises one or more IL15 proteins.

Pharmaceutical compositions of the binding compounds used in accordance with the present invention are prepared for storage by mixing proteins having the desired degree of purity with optional pharmaceutically acceptable carriers, excipients or stabilizers (see, e.g. Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). The formulation depends on the route of administration chosen. The binding compounds herein can be administered by intravenous injection or infusion or subcutaneously. For injection administration, the binding compounds herein can be formulated in aqueous solutions, preferably in physiologically-compatible buffers to reduce discomfort at the site of injection. The solution can contain carriers, excipients, or stabilizers as discussed above. Alternatively, binding compounds can be in lyophilized form for constitution with a suitable vehicle, e.g., sterile pyrogen-free water, before use.

Antibody formulations are disclosed, for example, in U.S. Pat. No. 9,034,324. Similar formulations can be used for the binding compounds of the present invention. Subcutaneous antibody formulations are described, for example, in US20160355591 and US20160166689.

### Methods of Use

The multispecific binding compounds and pharmaceutical compositions described herein can be used for the treatment of diseases and conditions characterized by the expression of PD-L1, including, without limitation, the conditions and diseases described above.

In one aspect, the multispecific binding compounds and pharmaceutical compositions herein can be used to treat cancers that are characterized by expression of PD-L1. As used herein, a cancer that is “characterized by expression of PD-L1” includes, without limitation, a cancer wherein one or more tumor cells express PD-L1, and/or wherein tumor-associated stroma exhibits expression of PD-L1, and/or wherein immune cells exhibit expression of PD-L1. Such disorders include, but are not limited to: invasive breast carcinoma, colon adenocarcinoma, lymphomas, lymphoid neoplasm diffuse large B-cell lymphoma, esophageal carcinoma, head and neck squamous cell carcinoma, lung adenocarcinoma, lung squamous cell carcinoma, ovarian serous cystadenocarcinoma, pancreatic adenocarcinoma, rectum adenocarcinoma, bladder urothelial carcinoma, cervical squamous cell carcinoma and endocervical adenocarcinoma, cholangio carcinoma, glioblastoma multiforme, hepatocellular carcinoma, mesothelioma, merkel cell carcinoma, renal cell carcinoma, sarcoma (e.g., undifferentiated sarcoma), skin cutaneous melanoma, stomach adenocarcinoma, testicular germ cell tumors, uterine carcinosarcoma, osteosarcoma, glioblastoma, melanoma, ovarian, gastric, and colorectal cancers.

Effective doses of the compositions of the present invention for the treatment of disease vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages can be titrated to optimize safety and efficacy.

Dosage levels can be readily determined by the ordinarily skilled clinician, and can be modified as required, e.g., as required to modify a subject's response to therapy. The amount of active ingredient that can be combined with the carrier materials to produce a single dosage form varies depending upon the host treated and the particular mode of administration. Dosage unit forms generally contain between from about 1 mg to about 500 mg of an active ingredient.

In some embodiments, the therapeutic dosage the agent may range from about 0.0001 to 100 mg/kg, and more usually 0.01 to 5 mg/kg, of the host body weight. For example, dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

Typically, compositions are prepared as injectables, either as liquid solutions or suspensions; solid forms suitable for solution in, or suspension in, liquid vehicles prior to injection can also be prepared. The pharmaceutical compositions herein are suitable for intravenous or subcutaneous administration, directly or after reconstitution of solid (e.g., lyophilized) compositions. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the antibodies and antibody structures described herein can be determined by standard pharmaceutical procedures in cell cultures or experimental animals, e.g., by determining the LD50 (the dose lethal to 50% of the population) or the LD100 (the dose lethal to 100% of the population). The dose ratio between toxic and therapeutic effect is the therapeutic index. The data obtained from these cell culture assays and animal studies can be used in formulating a dosage range that is not toxic for use in humans. The dosage of the antibodies described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The compositions for administration will commonly comprise an antibody or other agent (e.g., another ablative agent) dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the active agents and formulations thereof, of the invention and instructions for use. The kit can further contain a least one additional reagent, e.g., a chemotherapeutic drug, etc. Kits typically include a label indicating the intended use of the contents of the kit. The term “label” as used herein includes any writing, or recorded material supplied on or with a kit, or which otherwise accompanies a kit.

The invention now being fully described, it will be apparent to one of ordinary skill in the art that various changes and modifications can be made without departing from the spirit or scope of the invention.

### EXAMPLES

### Example 1: QL301 Binding to HEK293 Cells Expressing PD-L1 or 4-1BB

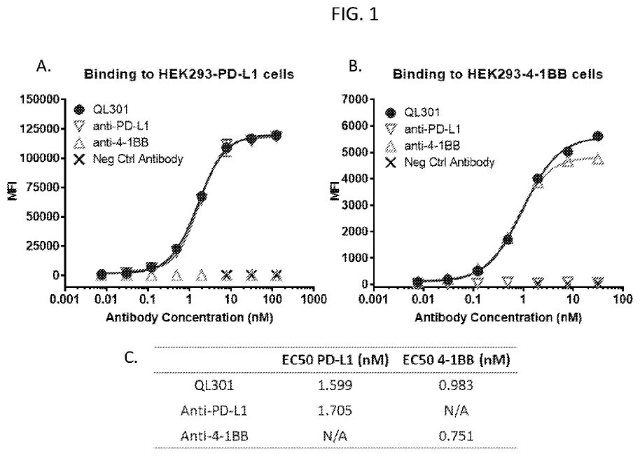

HEK293 cells expressing PD-L1 or 4-1BB were plated in a 96-well V-bottom plate at a density of 1×10⁵. Serially diluted antibodies were added to the cells and incubated for 30 min on ice. Following 2 washes with FACS buffer, AF647 labeled anti-human Fc secondary antibodies were added and incubated for 20 min on ice. Following 2 washes with FACS buffer, cells were resuspended in FACS buffer containing 7AAD viability dye and analyzed on a flow cytometer. The results are shown in FIG. 1, panels A-C.

### Example 2: Binding Kinetics

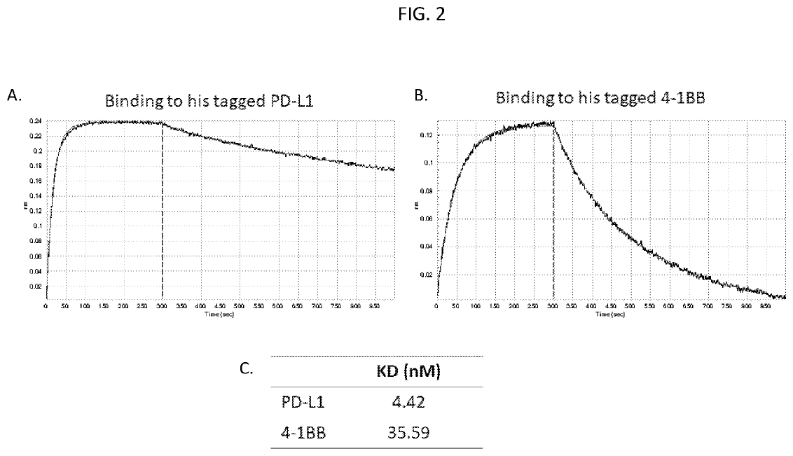

Binding kinetics were measured on the Octet RED96 system. Antibodies were loaded onto anti-human Fc capture (AHC) sensors from ForteBio followed by binding of the either his-tagged recombinant PD-L1 or 4-1BB proteins. The results are shown in FIG. 2, panels A-C.

### Example 3: QL301 Binding to HEK293 Cells Expressing Cyno PD-L1 or Cyno 4-1BB

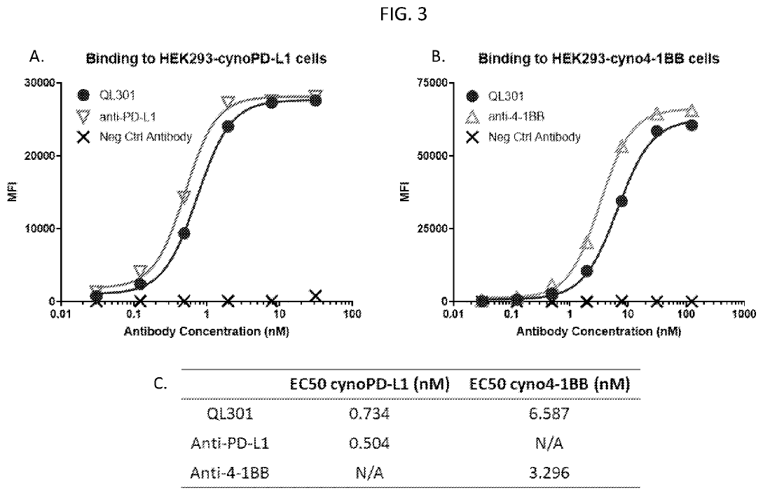

HEK293 cells expressing cynomolgus PD-L1 or 4-1BB were plated in a 96-well V-bottom plate at a density of 1×10⁵. Serially diluted antibodies were added to the cells and incubated for 30 min on ice. Following 2 washes with FACS buffer, AF647 labeled anti-human Fc secondary antibodies were added and incubated for 20 min on ice. Following 2 washes with FACS buffer, cells were resuspended in FACS buffer containing 7AAD viability dye and analyzed on a flow cytometer. The results are shown in FIG. 3, panels A-C.

### Example 4: QL301 Competition Assay

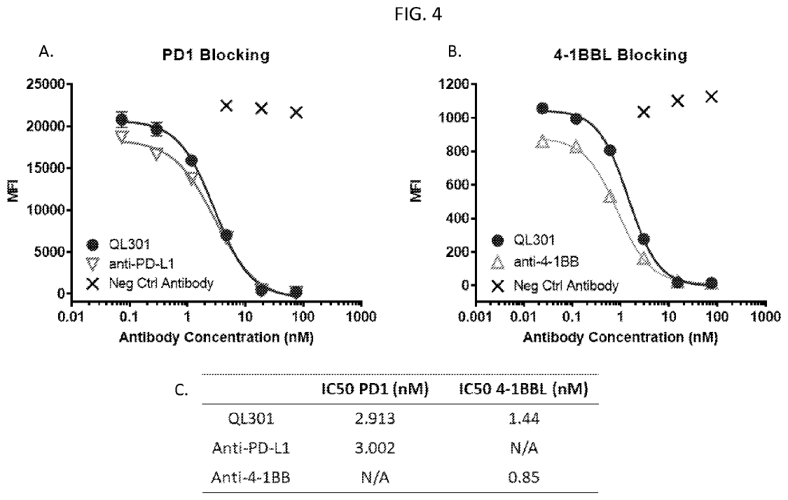

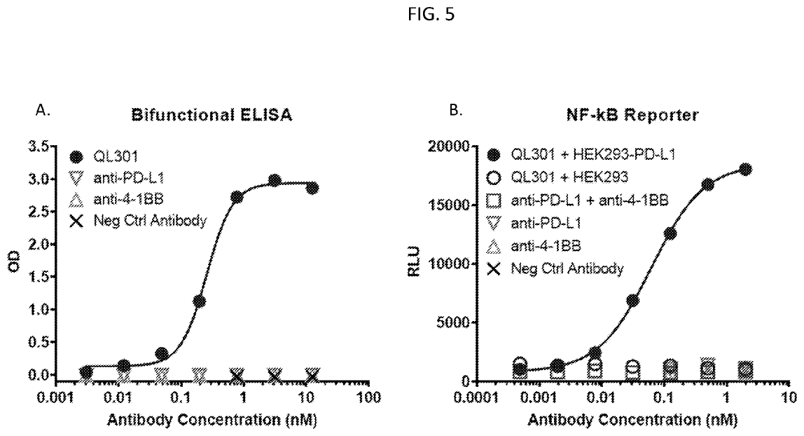

HEK293 cells expressing PD-L1 or 4-1BB were plated in a 96-well V-bottom plate at a density of 1×10⁵. Serially diluted antibodies were added to the cells and incubated for 15 min on ice. His-tagged recombinant PD-L1 or 4-1BB proteins were added to the respective plate and incubated for an additional 15 min on ice. Following 2 washes with FACS buffer, APC labeled anti-His-tag secondary antibodies were added and incubated for 20 min on ice. Following 2 washes with FACS buffer, cells were resuspended in FACS buffer containing 7AAD viability dye and analyzed on a flow cytometer. The results are shown in FIG. 4, panels A-C.

### Example 5: QL301 Bifunctional ELISA and NF-kB Reporter Assay

Recombinant His-tagged 4-1BB protein was coated onto a 96-well plate overnight at room temperature with shaking. After washing the plate with PBS containing 0.05% Tween-20, the plate was blocked with 2% BSA for 60 min and antibodies were added and incubated for 60 min at room temperature with shaking. After washing, biotinylated recombinant PD-L1 protein was added to the plate and incubated for 60 min at room temperature. The plate was washed and HRP (horseradish peroxidase) conjugated streptavidin was added and incubated for 30 min at room temperature. After washing, TMB (3,3′,5,5′-tetramethylbenzidine) substrate was added and incubated for 5 to 10 min to develop color, after which 0.16 M sulfuric acid was added to stop the reaction. Absorbance was read on a plate reader. For the reporter assay, HEK293 cells expressing 4-1BB that also contain a Renilla luciferase reporter element under NF-kB transcriptional control were seeded at 5×10⁴ cells per well in a 96-well plate. Parental HEK293 cells or HEK293 cells expressing PD-L1 were added at the same cell number per well. Serially diluted antibodies were then added and incubated for 24 h at 37° C. with 5% CO₂. Supernatant was then collected, transferred to a white wall 96-well plate, and QuantiLuc reagent (Invivogen) was added. Luminescence was read right away on a plate reader. The results are shown in FIG. 5, panels A-B.

### Example 6: Cytokine Release

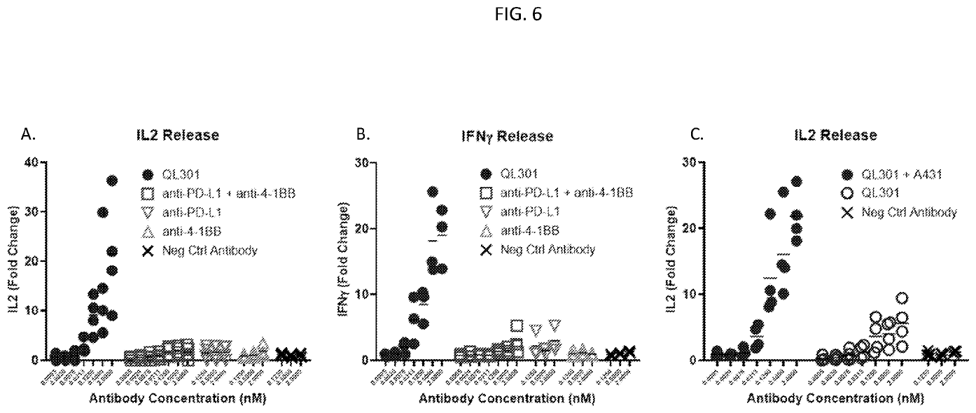

Human PBMCs were stimulated with anti-CD3 (OKT3) and incubated with QL301, PD-L1 or 4-1BB monoclonal, or their combination, along with PD-L1+ A431 cells. QL301 induced IL2 and IFN⋅ release, while anti-PD-L1 or 4-1BB alone, or the combination of the two, did not. In the absence of A431 cells, IL2 induction was significantly less. The results are shown in FIG. 6, panels A-C. Each dot represents an individual donor and values are fold over control antibody.

### Example 7: Cytokine Release in SEB Stimulation Assay

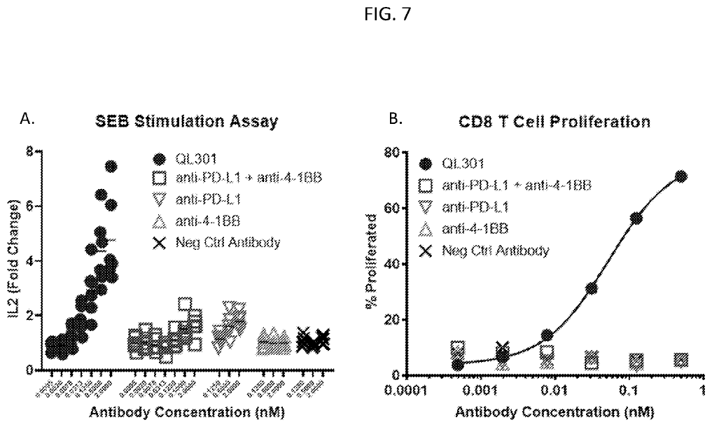

IL2 release was also observed in an SEB stimulation assay in the presence of QL301, but not PD-L1 or 4-1BB monoclonal, or the combination of the two. QL301 induced CD8+ T-cell proliferation in the presence of anti-CD3 (OKT3) and PD-L1+ A431 cells, but this was not observed with PD-L1 or 4-1BB monoclonal, or the combination of the two. The results are shown in FIG. 7, panels A-B.

### Example 8: MC38 Tumor Model

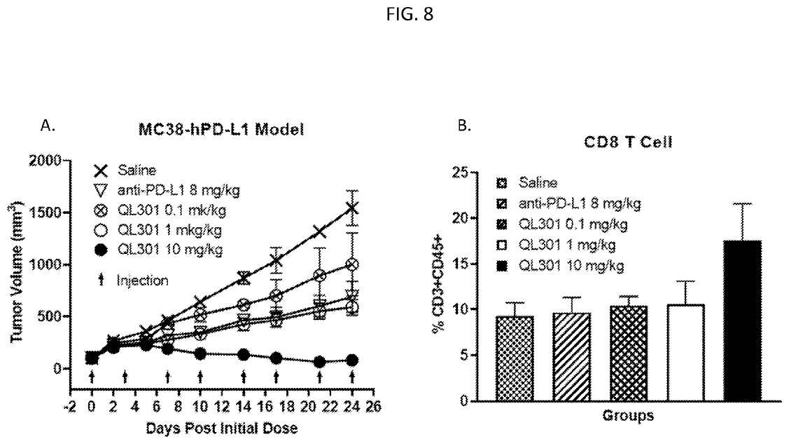

MC38 mouse cancer cells expressing human PD-L1 were implanted in the flanks of human PD-L1 and 4-1BB double knock-in C57BL/6 mice. QL301, PD-L1 monoclonal, or saline were administered i.p. twice weekly after the average tumor volume had reached around 100 mm³. QL301 at 10 mg/kg is significantly more efficacious than PD-L1 monoclonal antibody at the equal molar dose of 8 mg/kg (p<0.0001, n=6). Analysis of tumor infiltrating immune cells at the end of study showed more CD8+ T-cells in the tumors of animals that received QL301 compared to saline or PD-L1 monoclonal (p<0.01). The results are shown in FIG. 8, panels A-B.

### Example 9: A431 Tumor Model

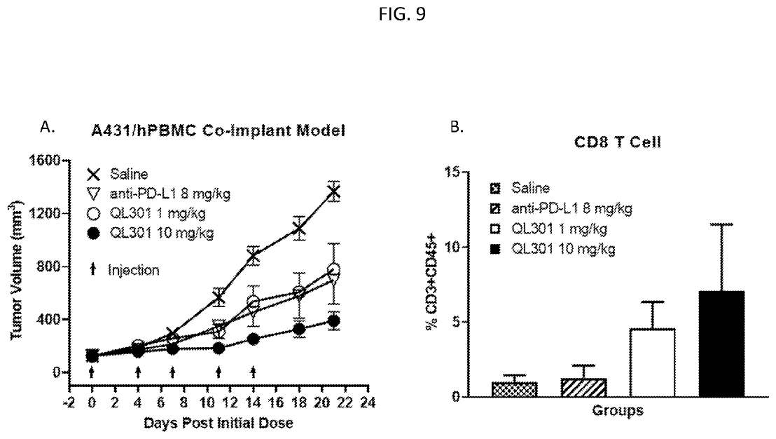

In a second model, A431 human cancer cells were co-implanted with human PBMCs in the flanks of CB17-SCID mice. Consistent with results from the MC38-hPD-L1 model, QL301 had better tumor growth inhibitory effect than the PD-L1 monoclonal antibody with higher percentage of CD8+ T-cells in the tumor (n=8). The results are shown in FIG. 9, panels A-B.

### Example 10: Accelerated Temperature Stress Test

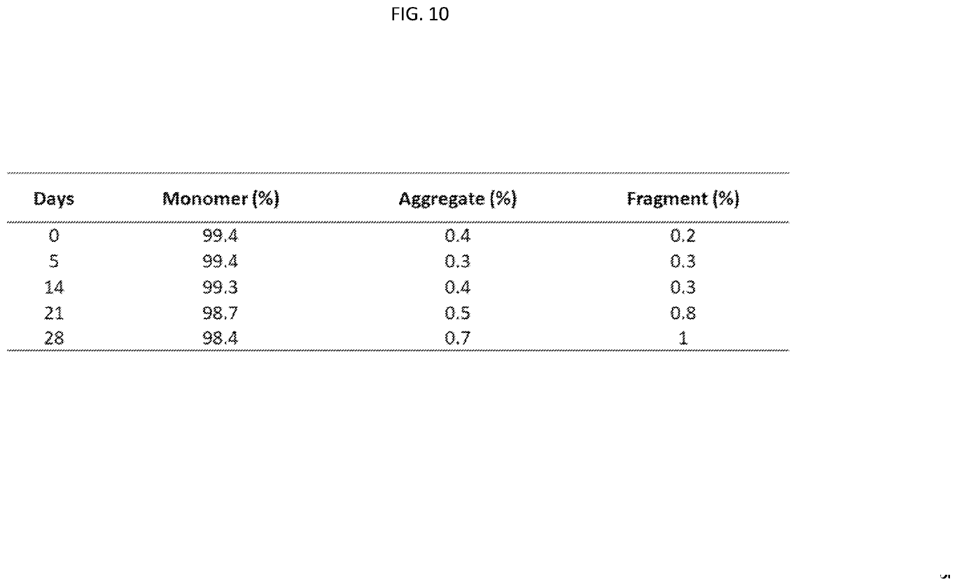

In an accelerated temperature stress test over the course of 28 days at 42° C., there was minimal change in the HPLC-SEC profile of QL301 (overlaid chromatogram of five time points). The calculated percentages of monomer, aggregate and fragment remained within 1% of initial production composition. The results are shown in FIG. 10.

### Example 11: Incubation of QL301 in Human Serum

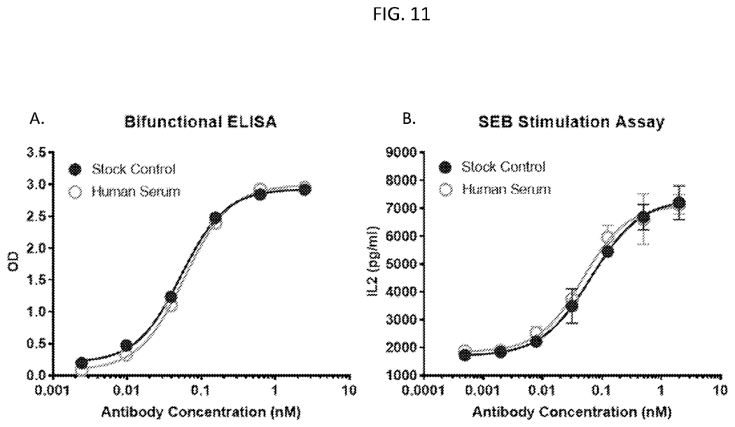

QL301 was incubated in human serum for 7 days and tested for binding and the ability to stimulate IL2 release from PBMCs in an SEB stimulation assay. No significant change was observed between the stock control at 4° C. and the serum-incubated molecule. The results are shown in FIG. 11, panels A-B.

### Example 12: ELISA Binding to PD-L1 and CD47

ELISA binding to PD-L1 and CD47 was evaluated. Immulon HBX plates were coated with 2 μg/mL hPDL1-FC (R&D Systems) overnight at 4° C. Plates were then washed 3 times with PBST and blocked for 1 hour at room temperature with 4% NFDM/PBS. Block was removed and antibody dilutions in 4% NFDM/PBS were added and incubated at room temperature for 1 hr. The plates were then washed 3 times with PBST. 1 μg/mL huCD47-C33S_his was added to each well and incubated for 1 hr. at RT, followed by washing 3 times with PBST. Anti-His-HRP (AbCam, 1:20,000) was added to each well and incubated at RT for 45 minutes. After washing 6 times with PBST, the assay was developed with TBM, followed by 2N sulfuric acid. The results are shown in FIG. 12, panel B. FIG. 12, panel A, is a schematic illustration of a PD-L1×CD47 bispecific antibody.

### Example 13: Binding of PD-L1×CD47 Bispecific Antibodies to HEK293 Cells

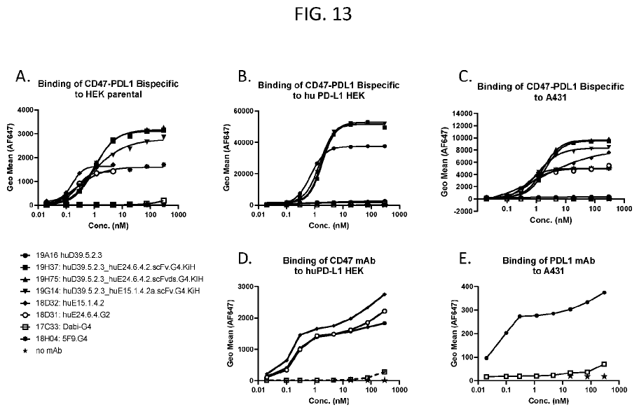

Cells were harvested and washed one time with FACS buffer. 1×10⁵ cells per well were distributed in 96-well v-bottom plates. Serial dilutions of test antibodies were added and incubated on ice for 20 minutes. The cells were washed 2 times with 200 μL FACS buffer. Next, the cells were resuspended in 50 μL of secondary antibody, AF647 F(ab′)² Goat anti-hu IgG, Fc specific at 1:500 dilution (Jackson ImmunoResearch, cat #109-606-098) and incubated on ice for 15 minutes. Finally, the cells were washed 2 times with 200 μL FACS buffer and resuspending in 100 μL of FACS buffer containing 7-AAD. The results are shown in FIG. 13, panels A-E.

### Example 14: PD-1 Fc-Biotin Blocking

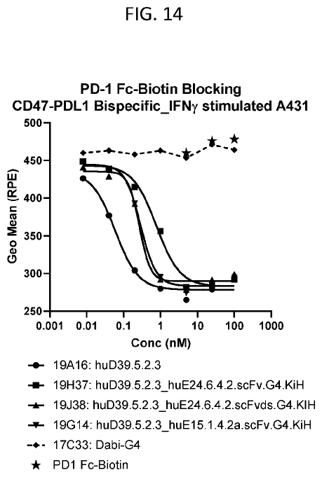

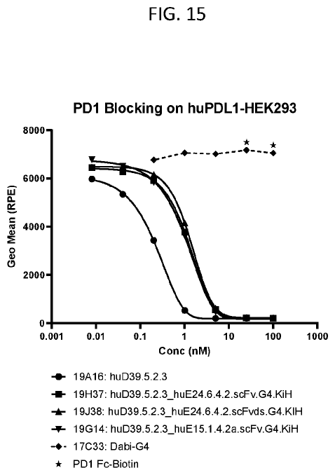

Cells were harvested and washed one time with FACS buffer. 1.2×10⁵ cells per well were distributed in 96-well v-bottom plates. 0.5 μg/mL PD-1-biotin (final concentration) were added to the cells and incubated for 5 minutes on ice. Next, serial dilutions of test antibodies were added and incubated on ice for 20 minutes. The cells were washed 2 times with 200 μL FACS buffer. Next, the cells were resuspended in 50 μL of secondary antibody, Streptavidin-APC (R&D, cat #F0050) at 10 μL/10⁶ cells and incubated on ice for 15 minutes. Finally, the cells were washed 2 times with 200 μL FACS buffer and resuspending in 120 μL of FACS buffer containing 7-AAD. The results are shown in FIG. 14 and FIG. 15.

### Example 15: SIRP⋅ Fc-Biotin Blocking

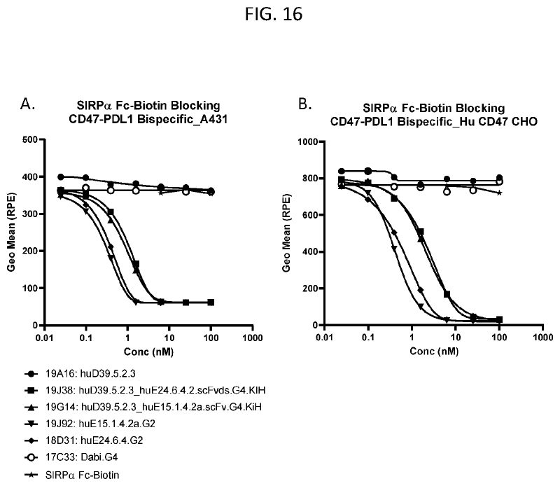

Cells were harvested and washed one time with FACS buffer. 1.2×10⁵ cells per well were distributed in 96-well v-bottom plates. 1.25 μg/mL SIRP⋅-biotin (final concentration) were added to the cells and incubated for 5 minutes on ice. Next, serial dilutions of test antibodies were added and incubated on ice for 20 minutes. The cells were washed 2 times with 200 μL FACS buffer. Next, the cells were resuspended in 50 μL of secondary antibody, Streptavidin-APC (R&D, cat #F0050) at 10 μL/10⁶ cells and incubated on ice for 15 minutes. Finally, the cells were washed 2 times with 200 μL FACS buffer and resuspending in 120 μL of FACS buffer containing 7-AAD. The results are shown in FIG. 16, panels A-B.

### Example 16: PD-L1/CD47 Mediated Phagocytosis of Raji and MM.1S Cells

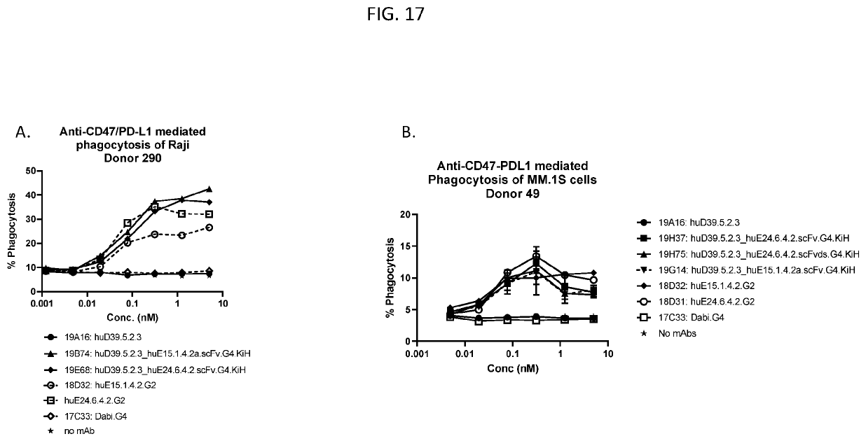

Recombinant human M-CSF (Miltenyi Biotec, cat #130-096-492) and recombinant human IL-10 (Miltenyi Biotec, cat #130-098-448) derived macrophages were generated from freshly isolated human peripheral blood mononuclear cells (PBMCs). Rh M-CSF (20 ng/ml) was added to the adherent cells in tissue culture flask after removing non-adherent cells on day 0, replenished with fresh medium on day 3 and day 7 and Rh IL-10 (10 ng/ml) was added on day 7 and further incubated for another 2 days in RPMI-1640 with 10% heat-inactivated FBS. CFSE labelled target cells (1×10⁵ cells/well) and Effector cells (2.5×10⁴ cells/well) were then incubated for 2 hours in 5% CO₂ incubator at 37° C. with serial dilutions of test antibodies in 96 wells ultra-low attachment u-bottom plates (Costar, cat #7007). Next, cells were transferred to 96-well v-bottom PP plates, spun down to pellet and washed one time with DPBS with 20% heat-inactivated FBS. Cells were then resuspended with DPBS with 20% HI FBS. APC conjugated anti-human CD36 antibody (ThermoFisher Scientific, cat #MA1-10210) was added to wells containing test antibodies and incubated on ice for 30 minutes. Cells were washed 2 times with 200 μL DPBS with 20% HI FBS. Finally, cells were resuspended with buffer containing 7-AAD. Samples were analyzed by flow cytometry using BD LSR Fortessa and further analyzed using FlowJo gating on Live CFSE/APC double positives indicating phagocytosis of target cells with macrophages induced by CD47-PDL1 antibodies. The results are shown in FIG. 17, panels A-B.

### Example 17: PD-L1/CD47 Binding to Red Blood Cells

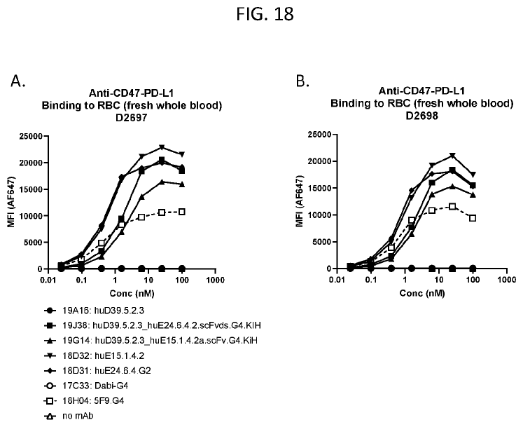

Human red blood cells (RBCs), obtained from buffy coat after isolation of mononuclear cells by density gradient centrifugation, were carefully transferred into 50 mL conical tubes. RBCs were washed 3 times with DPBS, and supernatant carefully removed after centrifugation. Then DPBS was added to make 10% solution of red blood cells. 1×10⁶ cells per well were distributed into 96-well v-bottom plates. Serial dilutions of test antibodies were added and incubated at 4° C. for 30 minutes. RBC was then washed 2 times with 200 μL DPBS with 2% FBS and 0.05% sodium azide (FACS buffer). Next, RBC was resuspended in 100 μL of secondary antibody AF647 F(ab′)2 goat anti-hu IgG, Fc specific at 1:500 dilution (Jackson ImmunoResearch, cat #109-606-098) and incubated at 4° C. for 15 minutes. Finally, RBC was washed 2 times with 200 μL FACS buffer and resuspended with 200 μL FACS buffer for flow cytometric analysis. The results are shown in FIG. 18, panels A-B.

### Example 18: Hemagglutination of Red Blood Cells Induced by CD47 Antibodies

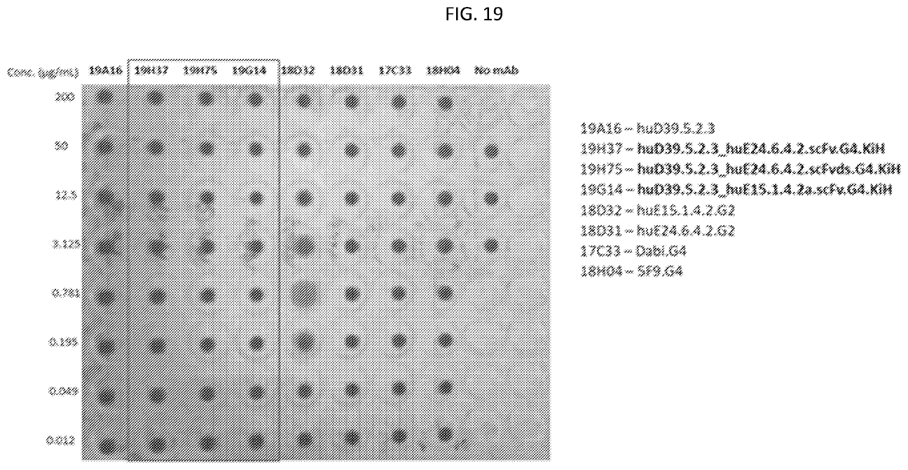

Fresh whole blood obtained from Stanford Blood Center was diluted with DPBS at 1:1 ratio. Then 2 μL of diluted blood was distributed into 96-well u-bottom plates. 50 μL of serially diluted test antibodies were added and incubated at room temperature for 2 hours. Picture files were taken of results. The results are shown in FIG. 19.

### Example 19: A431/hPBMC Co-Graft Tumor Model in ICR-SCID Mice

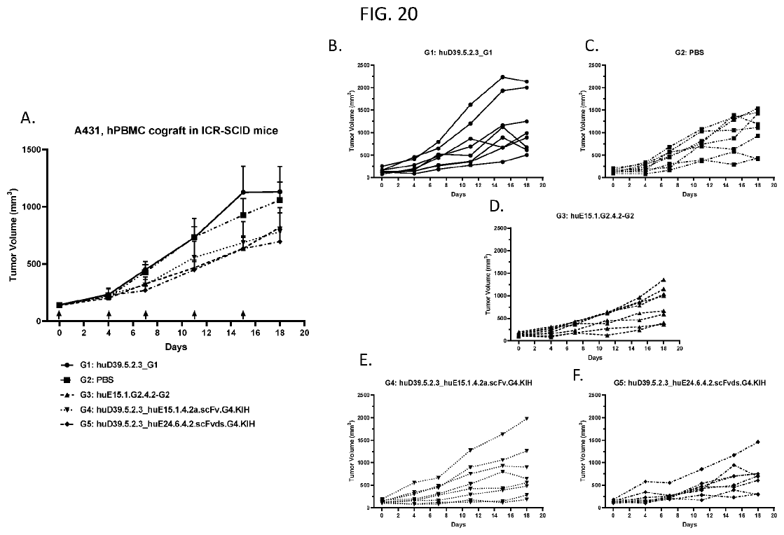

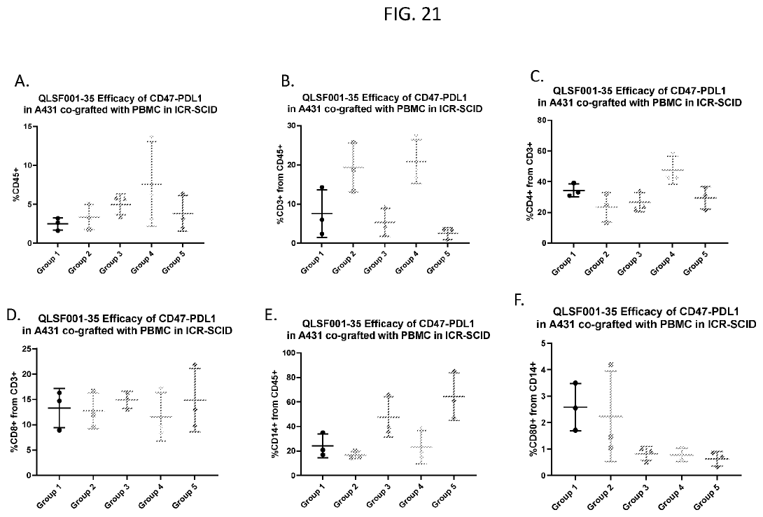

ICR-SCID mice were implanted subcutaneously with a mixture of A431 cells and human PBMC with matrigel so that each mouse received 5×10⁶ A431 cells and 1.5×10⁷ human PBMC. When the tumors reached an average of 140 mm³, the mice were dosed with test antibodies at 10 mg/kg IP or an equal volume of PBS on days 0, 4, 7, 11, and 15 (n=8). Tumor growth and mouse weight was monitored twice weekly. On day 18, tumors were collected and analyzed for lymphocyte and monocyte content. The results are shown in FIG. 20, panels A-F and FIG. 21, panels A-F.

### Example 20: A431 Tumor Model in NOD-SCID Mice

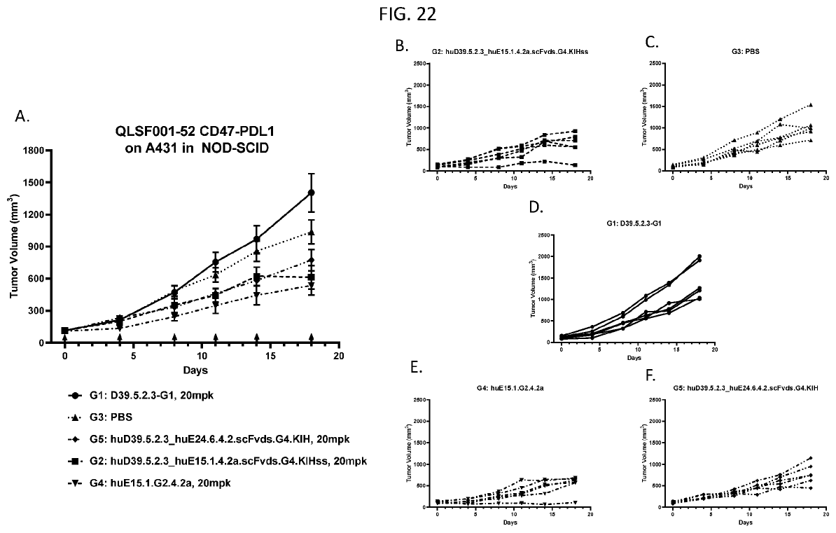

NOD-SCID mice were implanted subcutaneously with 5×10⁶ A431 cells per mouse. When the tumors reached an average of 110 mm³, the mice were dosed with test antibodies at 20 mg/kg IP or an equal volume of PBS on days 0, 4, 8, 11, 14, and 18 (n=6). Tumor growth and mouse weight was monitored twice weekly. The results are shown in FIG. 22, panels A-F.

### Example 21: PDL1-IL15 Antibodies Bind to Cells Expressing Human or Cynomolgus PD-L1 or Human IL2R⋅ and Human IL2R⋅

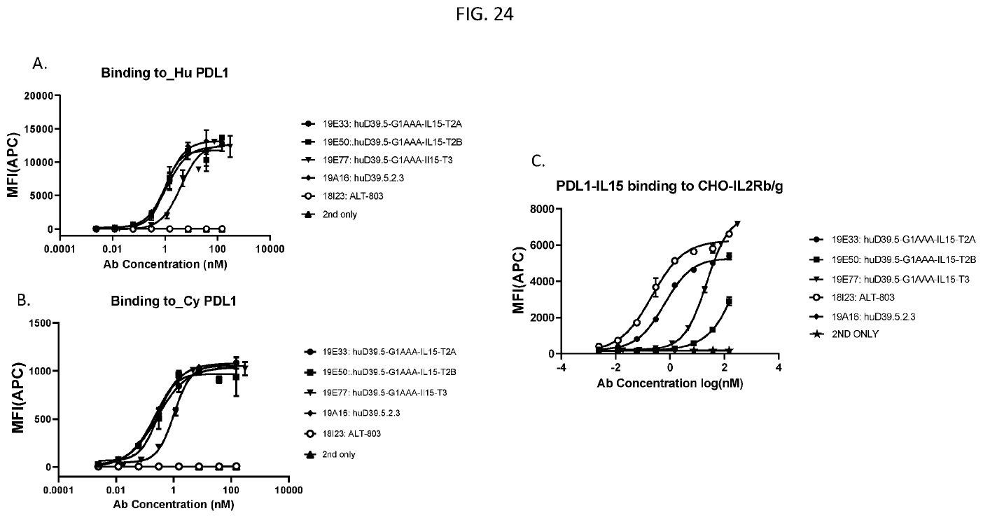

Cells were harvested and washed two times with FACS buffer. 2×10⁵ cells per well were distributed in 96-well v-bottom plates. Serial dilutions of test antibodies were added and incubated on ice for 20 minutes. The cells were washed 2 times with 200 μL FACS buffer. Next, the cells were resuspended in 50 μL of secondary antibody, AF647 F(ab′)2 Goat anti-hu IgG, Fc specific at 1:500 dilution (Jackson ImmunoResearch, cat #109-606-098) and incubated on ice for 25 minutes. Finally, the cells were washed 2 times with 200 μL FACS buffer and resuspending in 100 μL of FACS buffer containing 7-AAD. The results are shown in FIG. 24, panels A-C.

### Example 22: Proliferation of NK92 or M07e Cells in Response to PD-L1-IL15 Antibodies

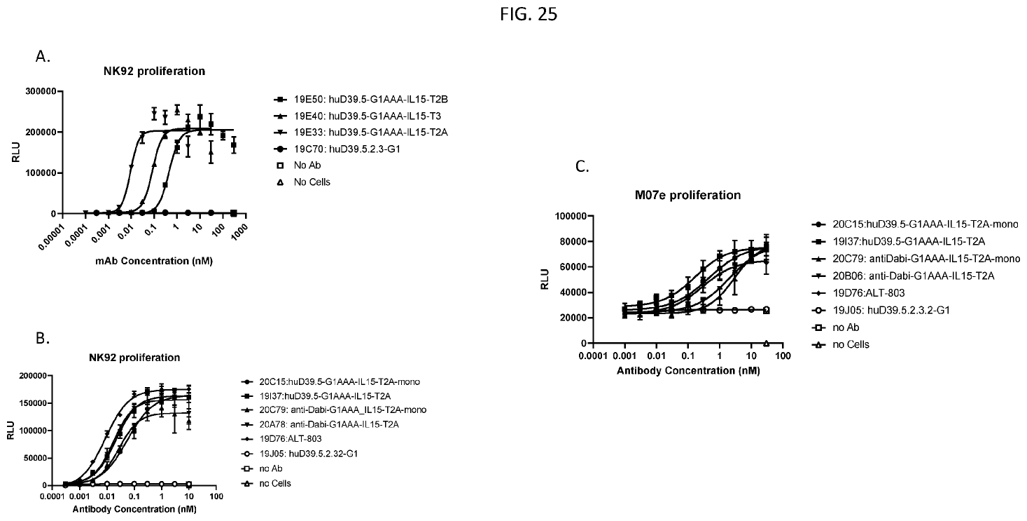

NK92 cells were cultured in MEM-alpha medium (Gibo, 12561056) supplemented with 12.5% horse serum, 12.5% fetal bovine serum, 0.2 mM inositol, 0.02 mM folic acid, 0.1 mM 2-mercaptoethanol, and 100-200 U/ml IL-2 (PeproTech). M07e cells were cultured in IMDM (Gibco, 12440046) supplemented with 20% fertal bovine serum and 10 ng/ml GM-CSF. For these proliferation assays, cells were harvested and washed two times with appropriate media not containing IL2 or GM-CSF. The cells were distributed at 20,000 cells/well in white 96-well plates and starved for 4 hours at 37° C. in 5% CO₂. Serial dilution of test antibodies was then added, and the plates were incubated for an additional 3 days. Proliferation was measured with CellTiter-Glo reagent (Promega) according to the manufacturer's instruction. Luminescence was recorded with a FlexStation3. The results are shown in FIG. 25, panels A-C.

### Example 23: Induction of pSTAT5 on M07e Cells with PD-L1-IL15 Antibodies

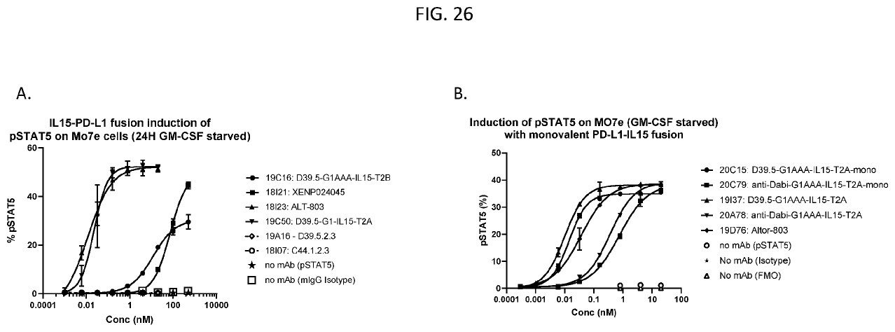

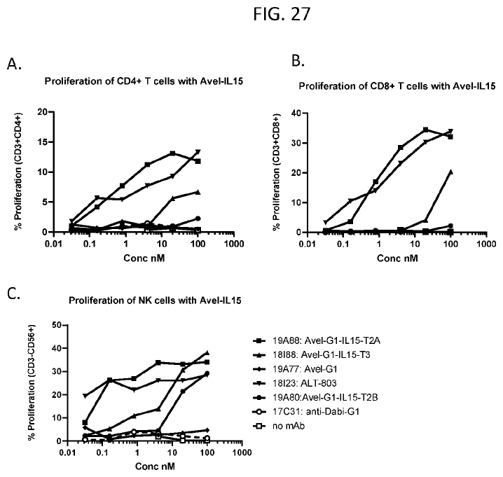

M07e cells were harvested and washed two times with IMDM media supplemented with 20% FBS not containing GM-CSF. Cells were starved of GM-CSF for 24 hours at 37° C. in 5% CO₂, then distributed at 1.5×10⁵ cells per well in 96-well v-bottom plates. Serial dilutions of the test antibodies were added and incubated for 20 minutes at 37° C. in 5% CO₂. For wells stimulated with GM-CSF as a positive control, the GM-CSF was added after 10 minutes, for a total of 10 minutes of stimulation. When incubation was complete, cells were fixed by adding 4% paraformaldehyde directly into the culture medium for a final concentration of 1.5% paraformaldehyde and incubated at room temperature for 10 minutes. Cells were then permeabilized by adding 100 μL ice-cold methanol and mixed with vigorous pipetting. After incubation for 10 minutes at 4°, cells were washed twice with staining buffer (PBS with 1% BSA), then resuspended with 50 μL staining buffer containing human Fc block. Next, anti-pSTAT5 antibody (AF647 mouse anti STATS pY694, BD cat #612599) or isotype control (mouse IgG1 isotype control, BD, cat #557714) was added and incubated at room temperature for 15 to 30 minutes. Cells were then washed two times with staining buffer and resuspended in 120 uL staining buffer for analysis on LSF Fortessa. The results are shown in FIG. 26, panels A-B.

### Example 24: PD-L1-IL15 Antibodies Increase Proliferation of CD4+ and CD8+ T-Cells, NKT Cells, and NK Cells

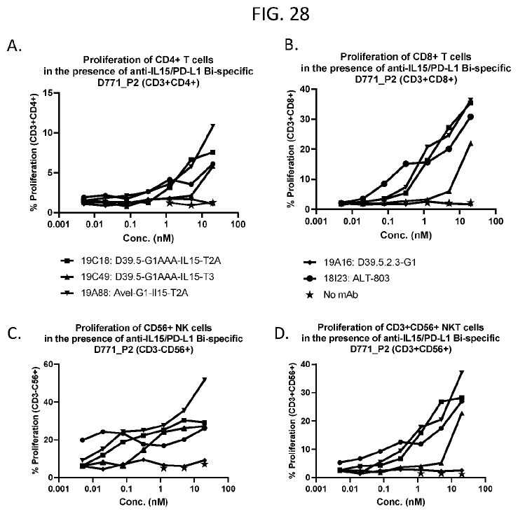

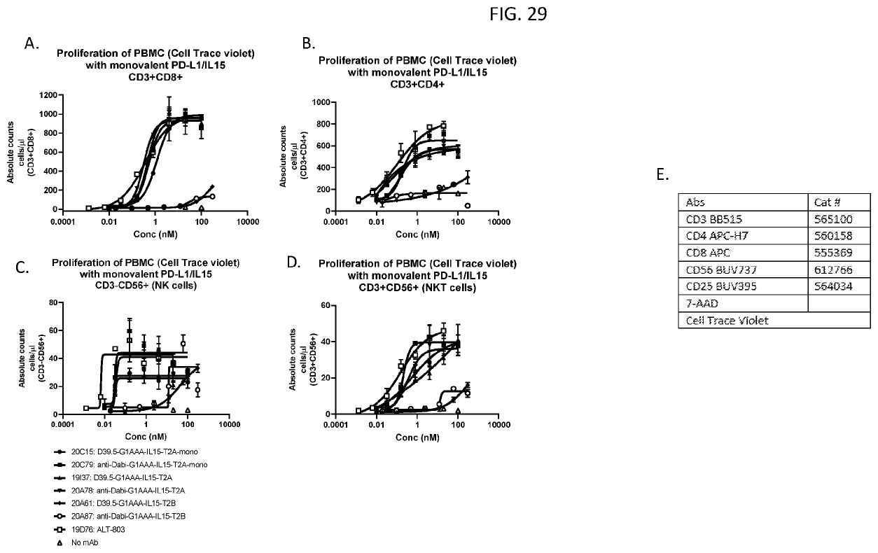

PBMCs were isolated according to the Miltenyi Biotech Density Centrifugation protocol. Red blood cells were lysed with RBC lysis buffer (eBiosciences) according to the manufacturer's protocol. After isolation, PBMCs were washed once with PBS plus 2% FBS and resuspended at 2×10⁷ cells/mL. CellTrace Violet was prepared at 6 μM and added to the PBMCs for a final concentration of 3 μM. After incubation at room temperature in the dark for 10 minutes, an equal volume of FBS was added to stop the reaction. PBMCs were then washed twice with PBS plus 2% FBS and resuspended in RPMI with 10% heat inactivated FBS at 2×10⁶ cells/mL. PBMCs were then distributed at 2×10⁵ cells per well in 96-well plates. Serial dilutions of test antibodies were then added, and the plates were incubated at 37° C. in 5% CO₂ for five days. PBMCs were then analyzed for proliferation by staining with antibodies to the following human proteins with corresponding fluorophores: CD3-BB515, CD4-APC-H7, CD8-APC, CD56-BV786, and CD25 BUV395. The results are shown in FIG. 27, panels A-C, FIG. 28, panels A-D, and FIG. 29, panels A-E.

### Example 25: Pharmacodynamics of PD-L1-IL15 Antibodies on Murine Lymphocyte Counts

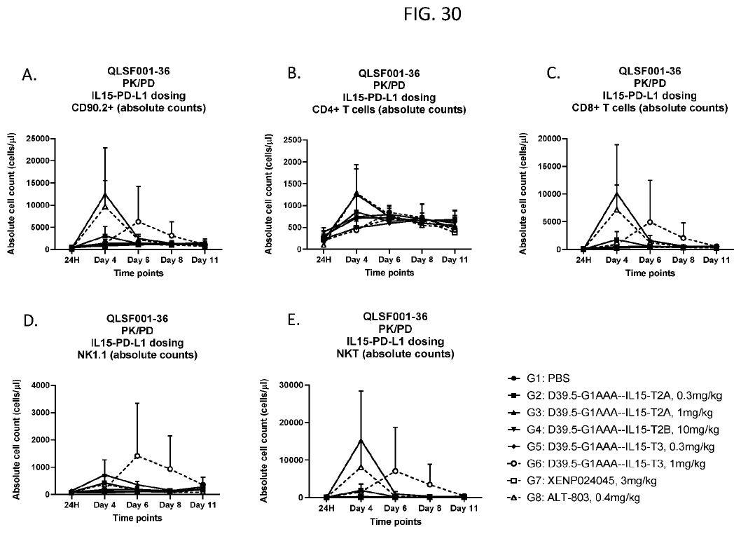

C57BL/6 mice were dosed IP with test antibodies or equal volume of PBS (n=3). Whole blood was collected on Days 1, 4, 6, 8, and 11. Mouse Fc block CD16/CD32 Clone 2.4G2 (BD Cat #553142) was added to 50 μL anti-coagulated whole mouse blood at 1.2-1.5 μL per 50 μL of blood and incubated at 4° C. for 5 minutes. Fluorochrome conjugated antibodies for mouse lymphocyte markers were mixed and added to the blood samples, which were then incubated at 4° C. for 15-20 minutes in the dark. After incubation, red blood cells were lysed with BD Lysing Buffer (BD, cat #555899) by adding 800 μL to each sample and vortexing vigorously. After a 15-minute incubation at room temperature in the dark, samples were centrifuged at 350×g for 5 minutes and the supernatant discarded. Cells were washed once with 2 mL BD stain buffer (BD cat #554657) and resuspending win 350 μL of BD stain buffer with 7-AAD and 50 μL of Counting Beads (Biolegend cat #424902) per sample. The results are shown in FIG. 30, panels A-E.

### Example 26: Pharmacodynamics of PD-L1-IL15 antibodies on murine lymphocyte counts

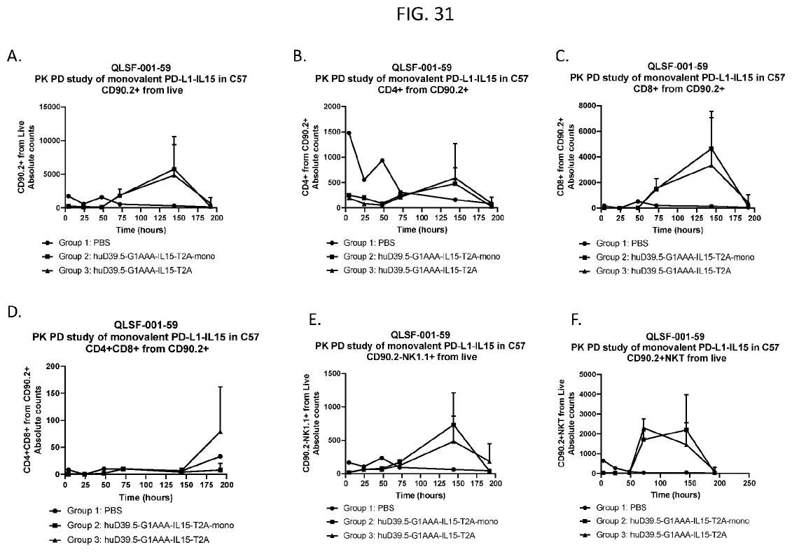

C57BL/6 mice were dosed IP with test antibodies (0.5 mg/kg) or equal volume of PBS (n=3). Whole blood was collected at 4 hours and at days 1, 2, 3, 6, and 8. Mouse Fc block CD16/CD32 Clone 2.4G2 (BD Cat #553142) was added to 50 μL anti-coagulated whole mouse blood at 1.2-1.5 μL per 50 μL of blood and incubated at 4° C. for 5 minutes. Fluorochrome conjugated antibodies for mouse lymphocyte markers were mixed and added to the blood samples, which were then incubated at 4° C. for 15-20 minutes in the dark. After incubation, red blood cells were lysed with BD Lysing Buffer (BD, cat #555899) by adding 800 μL to each sample and vortexing vigorously. After a 15-minute incubation at room temperature in the dark, samples were centrifuged at 350×g for 5 minutes and the supernatant discarded. Cells were washed once with 2 mL BD stain buffer (BD cat #554657) and resuspending win 350 μL of BD stain buffer with 7-AAD and 50 μL of Counting Beads (Biolegend cat #424902) per sample. The results are shown in FIG. 31, panels A-F.

### Example 27: Pharmacokinetic Determination of D39.5-G1AAA-IL15 Types T2A, T2B, T3 and T2A-Mono in C57Bl/6 and NSG Mice

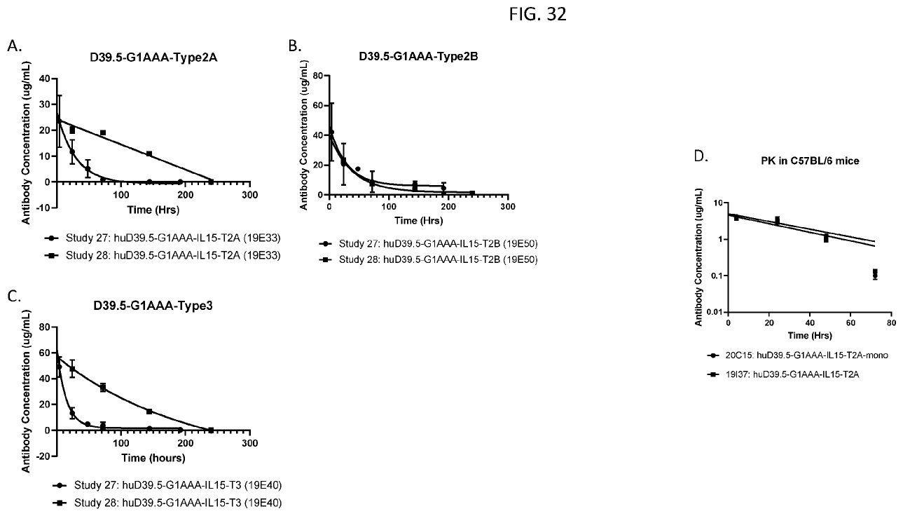

Panels A-C: Study 27: C57BL/6 mice (n=6, n=3 per time point) were dosed once IV on Day 0. Whole blood and plasma were collected at 4 hours and on Days 1, 2, 3, 6 and 8. Study 28: NSG mice were implanted with U118 cells (5×10⁶ cells/mouse) with Matrigel. When tumors had reached 250 mm³, each tumor bearing mouse was grouped with a non-tumor bearing NSG mouse. The PBS group had two non-tumor bearing mice. On Day 0, mice were injected IV with human PBMC (5×10⁶ cells/mouse). On Day1, mice received test antibodies or equal volume of PBS. Whole blood and plasma were collected on Days 2, 4, 7, and 11. Panel D: C57Bl/6 mice (n=6, n=3 per time point) were dosed once IP on Day 0. Whole blood and plasma were collected at 4 hours and on Days 1, 2, 3, 7 and 9. The results are shown in FIG. 32, panels A-D.

### Example 28: Tumor Growth Inhibition of MC38 Murine Colon Cancer Cells Expressing Human PD-L1 with PDL1-G1AAA-IL15-T2A

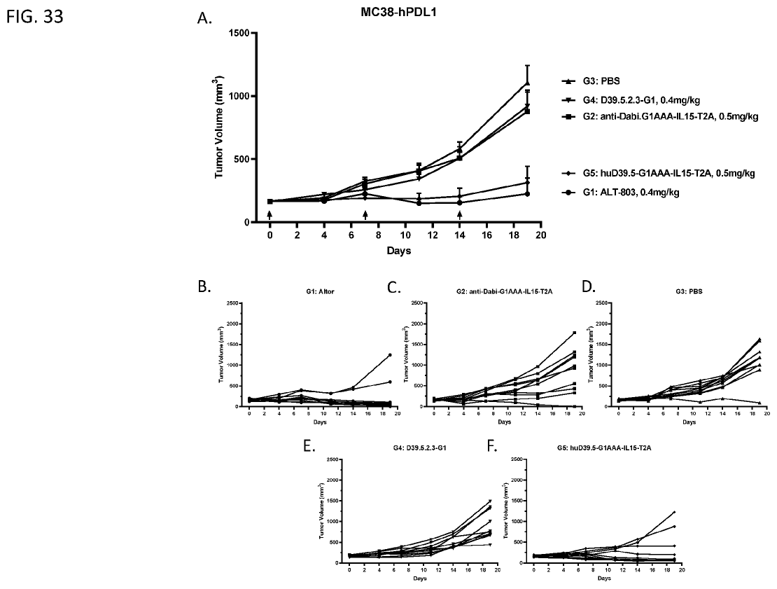

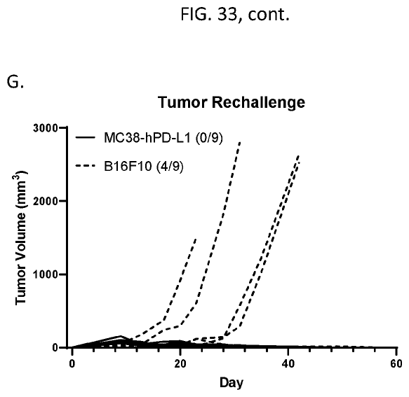

MC38-hPDL1 cells were implanted subcutaneously into C57BL/6 mice at 5×10⁶ cells per mouse with Matrigel. When tumors reached an average of 165 mm³, mice were randomized (n=10) and dosed IP with test molecules or an equal volume of PBS on days 0, 7, and 14. Tumor growth and body weight was monitored twice weekly. The results are shown in FIG. 33, panels A-F. Tumor free mice after PDL1-G1AAA-IL15-T2A treatment were rechallenged with either MC38-hPD-L1 or B16F10 cancer cells. Data are shown in FIG. 33, panel G. No MC38-hPD-L1 grew, suggesting lasting protective immune memory.

### Example 29: Tumor Growth Inhibition of A431 Xenograft Co-Grafted with Human PBMCs

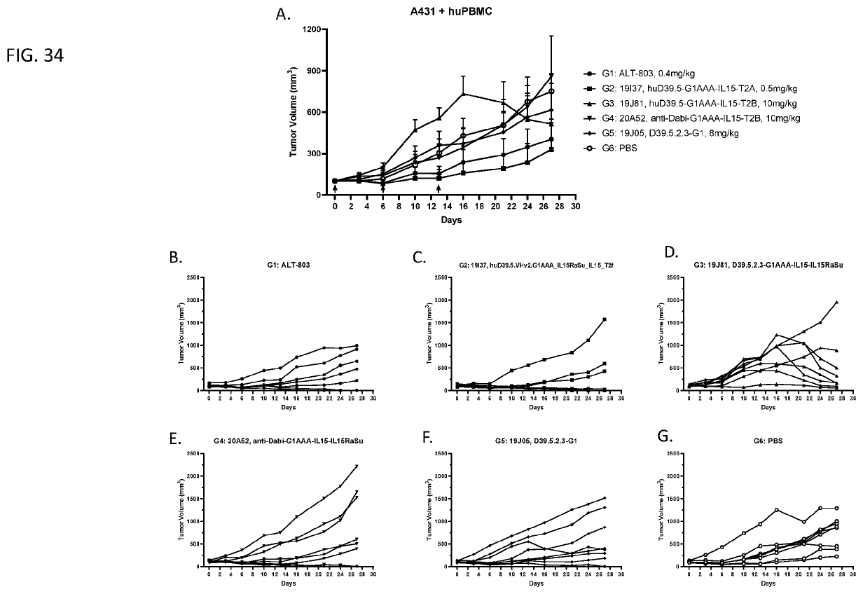

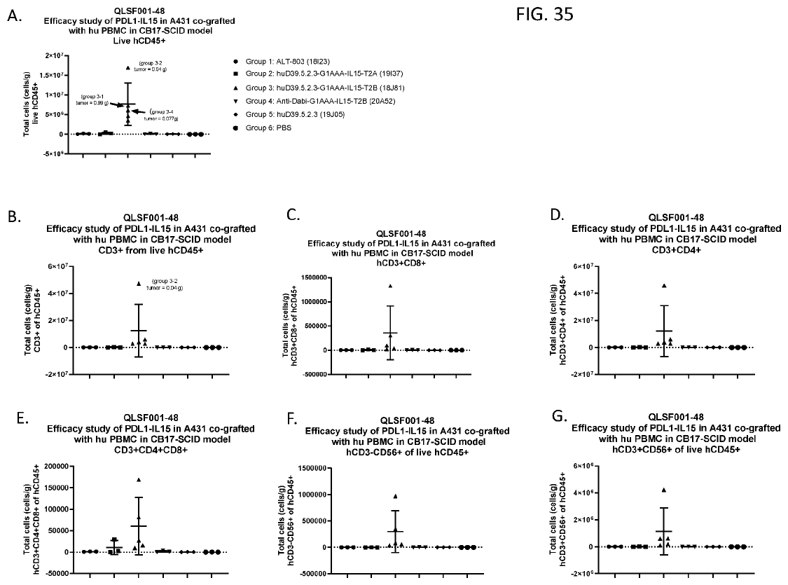

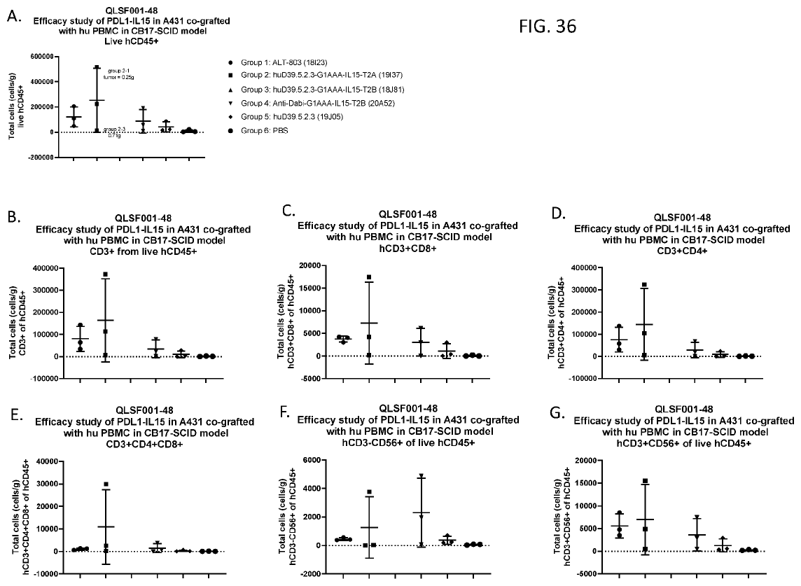

A431 cells (5×10⁶ cells/mouse) were mixed with human PBMCs (15×10⁶ cells/mouse) and Matrigel (1:1), then implanted subcutaneously into CB17-SCID mice. When tumors reached an average of 100 mm³, mice were randomized (n=8) and dosed IP with test molecules or an equal volume of PBS on days 0, 6, and 13. Tumor growth and body weight was monitored twice weekly. Mice were euthanized and tumors were harvested on Day 27. Tumors were homogenized and stained for human T-cell and NK cell markers CD45, CD3, CD8, CD4 and CD56. Samples were analyzed by flow cytometry using BD LSR Fortessa and further analyzed using FlowJo. The results are shown in FIG. 34, panels A-G. Data from the phenotype analysis of tumors are shown in FIG. 35, panels A-G and FIG. 36, panels A-G. There were significant increases in human T-cells, NK cells and NKT cells in groups 1, 2, 3, and 4.

### Example 30: Tumor Growth Inhibition of MC38 Murine Colon Cancer Cells Expressing Human PD-L1 with PDL1-G1AAA-IL15-T2A in C57BL/6 Mice

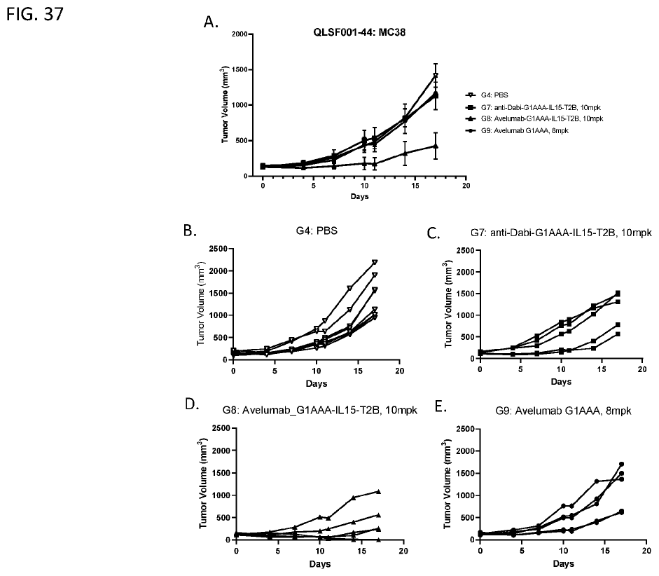

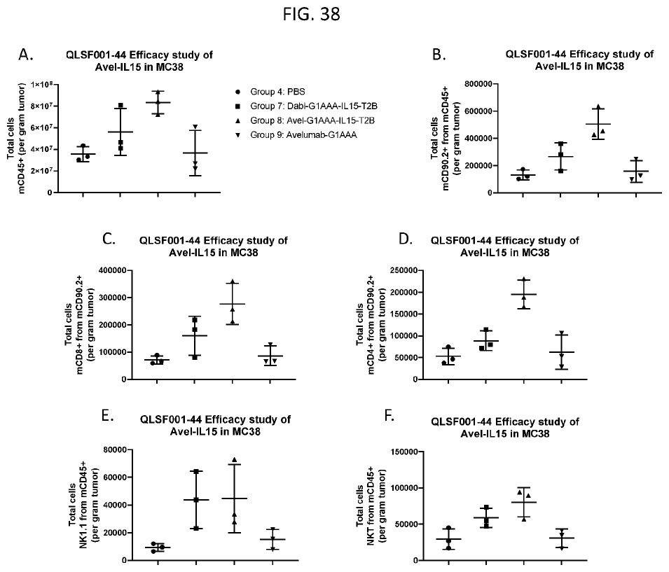

MC38 cells were implanted subcutaneously into C57BL/6 mice at 0.6×10⁶ cells per mouse with Matrigel. When tumors reached an average of 145 mm³, mice were randomized (n=5, n=8 for PBS) and dosed IP with test molecules or an equal volume of PBS on days 0, 7, and 14. Tumor growth and body weight was monitored twice weekly. Tumors were homogenized and stained for mouse T-cell and NK cell markers CD45, CD90.2, CD8, CD4 and NK1.1. Samples were analyzed by flow cytometry using BD LSR Fortessa and further analyzed using FlowJo. The results are shown in FIG. 37, panels A-E. Data from the phenotype analysis of tumors are shown in FIG. 38, panels A-F.

### Example 31: Tumor Growth Inhibition of NCI-H1650 Cells Co-Grafted with Human PBMCs in CB17-SCID Mice

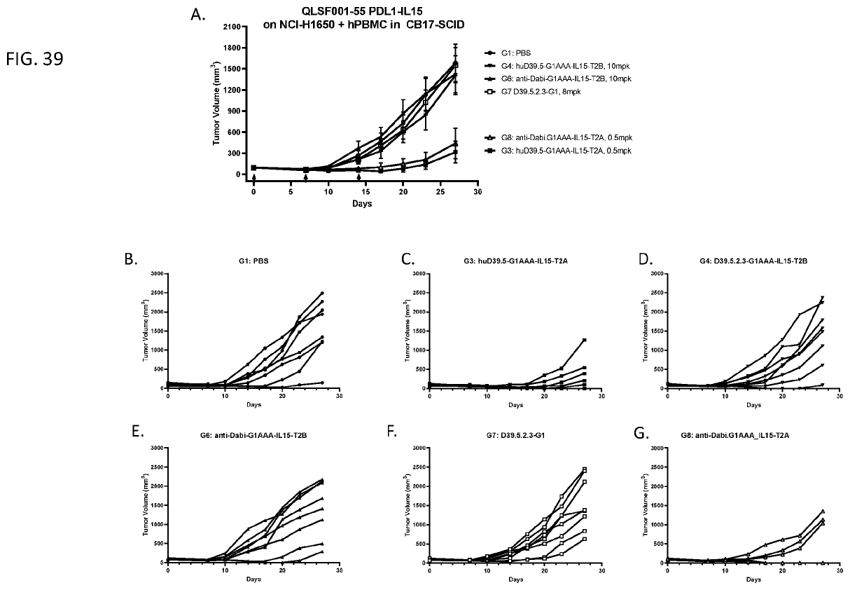

NCI-H1650 cells (10×10⁶ cells/mouse) were mixed with human PBMCs (10×10⁶ cells/mouse) and Matrigel (1:1), then implanted subcutaneously into CB17-SCID mice. When tumors reached an average of 95 mm³ mice were randomized (n=8) and dosed IP with test molecules or an equal volume of PBS on days 0, 7, and 14. Tumor growth and body weight was monitored twice weekly. The results are shown in FIG. 39, panels A-G.

### Example 32: Phagocytosis of Red Blood Cells (RBCs) Induced by CD47-PDL1 Bispecific Antibodies is Less than that of Monoclonal Anti-CD47 Antibodies

Recombinant human M-CSF and recombinant human IL-10 derived macrophages were generated as described in Example 16 and FIG. 17. Carefully isolated RBCs were labelled with 1 μM of CellTrace CFSE (ThermoFisher Scientific, cat #C34554). CFSE labelled RBC (1×10⁵ cells/well) and Macrophages (2.5×10⁴ cells/well) were incubated for 2 hours in 5% CO₂ at 37° C. with serial dilutions of test antibodies in 96 wells ultra-low attachment u-bottom plates (Costar, cat #REF7007). Cells were then transferred to 96-well v-bottom PP plates, spun down to pellet and washed one time with DPBS with 20% heat-inactivated FBS. Cells were resuspended with DPBS with 20% HI FBS and APC-conjugated anti-human CD36 antibody (ThermoFisher Scientific, cat #MA1-10210) was added to wells containing testing antibodies and incubated on ice for 20 minutes. Cells were washed 2 times with 200 μL DPBS with 20% HI FBS. Finally, cells were resuspended with buffer containing 7-AAD. Samples were analyzed by flow cytometry using BD LSR Fortessa and further analyzed using FlowJo gating on Live CFSE/APC double positives indicating phagocytosis of RBCs with macrophages induced by anti-CD47 antibodies. The results are shown in FIG. 41, panels A and B.

### Example 33: Binding of Type2A Masked Antibodies Before and After Cutting with MMP14 and uPA to CHOK1-IL2RbMg Cells

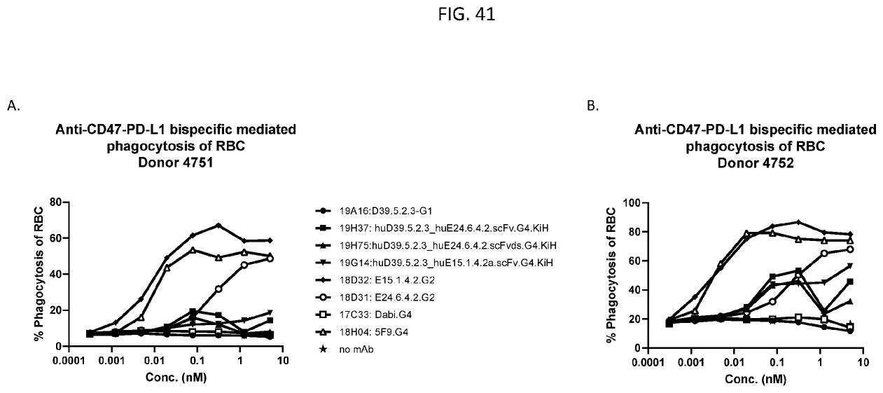

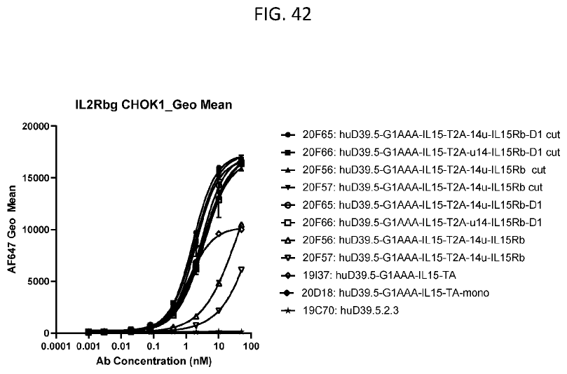

16 μg of each antibody was digested overnight at 37° with 0.4 μg furin activated MMP14 and 0.4 μg uPA supplemented with zinc chloride. After digestions, CHOK1-IL2Rb/g cells were harvested and washed two times with FACS buffer. 1×10⁵ cells per well were distributed in 96-well v-bottom plates. Serial dilutions of test antibodies (cut and uncut) were added and incubated on ice for 30 minutes. The cells were washed 2 times with 200 μL FACS buffer. Next, the cells were resuspended in 50 μL of secondary antibody, AF647 F(ab′)2 Goat anti-hu IgG, Fc specific at 1:500 dilution (Jackson ImmunoResearch, cat #109-606-098) and incubated on ice for 20 minutes. Finally, the cells were washed 2 times with 200 μL FACS buffer and resuspended in 120 μL of FACS buffer containing 7-AAD. The results are shown in FIG. 42, and indicate that antibodies using only D1 of IL15Rb do not reduce binding; however, masking with IL15Rb reduces binding by >10-fold.

### Example 34: Proliferation of NK92 Cells in Response to Type2A Masked Antibodies Before and After Cutting with MMP14 and uPA

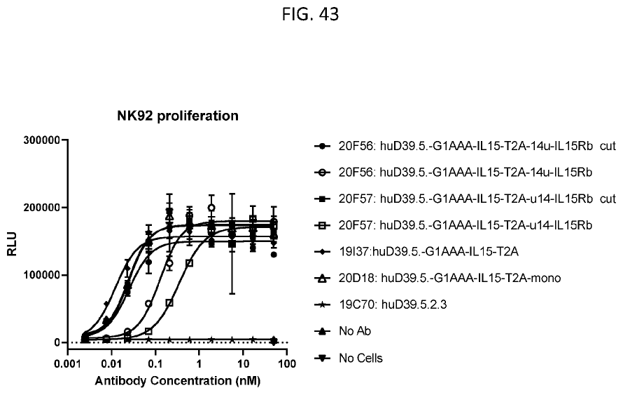

NK92 cells were cultured in MEM-alpha medium (Gibo, 12561056) supplemented with 12.5% horse serum, 12.5% fetal bovine serum, 0.2 mM inositol, 0.02 mM folic acid, 0.1 mM 2-mercaptoethanol, and 100-200 U/ml IL-2 (PeproTech). 16 μg of each antibody was digested overnight at 37° with 0.4 μg furin activated MMP14 and 0.4 μg uPA supplemented with zinc chloride. After digestions, NK92 cells were harvested and washed two times with culture media not containing IL2. The cells were distributed at 20,000 cells/well in white 96-well plates and starved for 4 hours at 37° C. in 5% CO₂. Serial dilutions of test antibodies were then added, and the plates were incubated for an additional 3 days. Proliferation was measured with CellTiter-Glo reagent (Promega) according to the manufacturer's instructions. Luminescence was recorded with a FlexStation3. The results are shown in FIG. 43, and indicate that masking with IL15Rb reduces proliferation by 5- to 15-fold.

### Example 35: AST and ALT Levels in Rhesus Monkeys in a 4-Week Repeated Dose Toxicology Study of PD-L1×4-1BB Bispecific Antibody

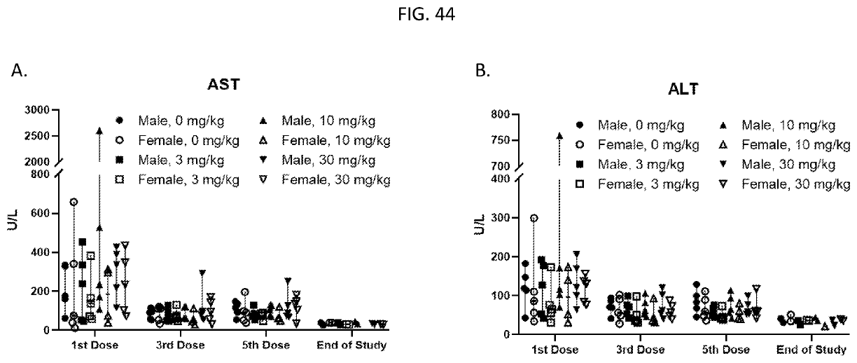

A 4-week repeated dose toxicology study of PD-L1×4-1BB bispecific antibody was conducted in rhesus monkeys, and asparate transaminase (AST) and alanine transaminase (ALT) levels were measured. The results are shown in FIG. 44, panels A and B. There was no chronic elevation in AST or ALT levels after repeated administrations, suggesting that PD-L1×4-1BB at 3, 10 and 30 mg/kg had minimal toxic effect on the liver.

### Example 36: A375 Tumor Growth Inhibition by PD-L1×CD47 (QL401) Bispecific Antibody in NOG Mice

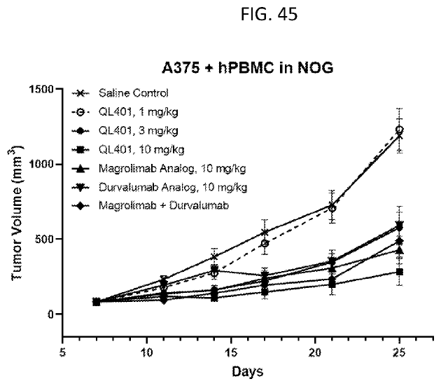

Human PBMCs were implanted into NOG mice prior to inoculation of A375 cells. The results from this tumor model are shown in FIG. 45. The anti-tumor effect of PD-L1×CD47 (QL401) at 10 mg/kg was comparable or more potent than magrolimab, durvalumab or their combination.

### Example 37: Raji Tumor Growth Inhibition by PD-L1×CD47 (QL401) Bispecific Antibody in NOG Mice

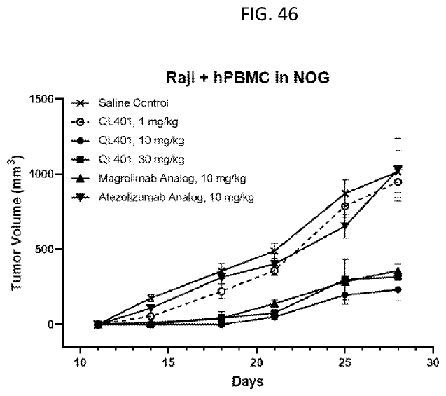

Human PBMCs were implanted into NOG mice prior to inoculation of Raji cells. The results from this tumor model are shown in FIG. 46. The anti-tumor effect of PD-L1×CD47 (QL401) at 10 mg/kg was comparable to magrolimab.

### Example 38: Red Blood Cell Count in Cynomolgus Monkeys in a 4-Week Repeated Dose Toxicology Study of PD-L1×CD47 Bispecific Antibody

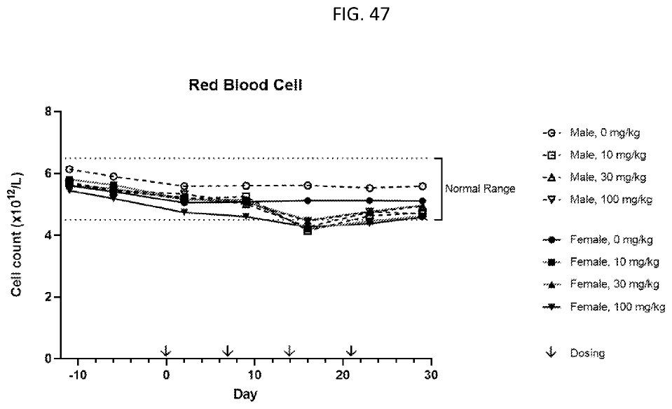

A 4-week repeated dose toxicology study was conducted with PD-L1×CD47 bispecific antibody in cynomolgus monkeys. The results from this model are shown in FIG. 47. Red blood cell count did not decrease significantly below the normal range after repeated administrations of PD-L1×CD47 at 10, 30, and 100 mg/kg doses.

### Example 39: Stimulation of cDC1 by a mouse cross-reactive surrogate of PDL1-G1AAA-IL15-T2A

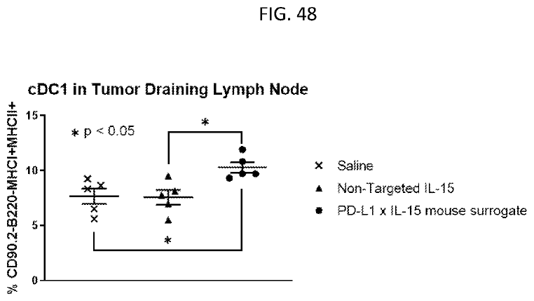

MC38 tumor cells were implanted in C57BL/6 mice and grown to −100 mm³. Mice were treated with saline, a non-targeted IL-15 fusion protein, and a mouse cross-reactive surrogate of PD-PDL1-G1AAA-IL15-T2A. Tumor-draining lymph nodes were collected and antigen presenting cells were analyzed by FACS. The results are shown in FIG. 48. The PD-L1×IL-15 surrogate molecule induced a higher percentage of conventional dendritic cell 1 (cDC1), suggesting a secondary mechanism of anti-tumor effect through stimulation of antigen presenting cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-114. (canceled)

115. A bispecific antibody that binds to PD-L1 and 4-1BB, comprising:
two binding units that bind to PD-L1, each comprising:
a heavy chain variable region comprising:
a CDR1 sequence comprising SEQ ID NO: 1;
a CDR2 sequence comprising SEQ ID NO: 2; and
a CDR3 sequence comprising SEQ ID NO: 3; and
a light chain variable region comprising:
a CDR1 sequence comprising SEQ ID NO: 7;
a CDR2 sequence comprising SEQ ID NO: 8; and
a CDR3 sequence comprising SEQ ID NO: 9; and
two binding units that bind to 4-1BB, each comprising a single chain Fv (scFv) comprising:
a heavy chain variable region comprising:
a CDR1 sequence comprising SEQ ID NO: 13;
a CDR2 sequence comprising SEQ ID NO: 14; and
a CDR3 sequence comprising SEQ ID NO: 15; and
a light chain variable region comprising:
a CDR1 sequence comprising SEQ ID NO: 19;
a CDR2 sequence comprising SEQ ID NO: 20; and
a CDR3 sequence comprising SEQ ID NO: 21.

116. The bispecific antibody of claim 115, wherein the CDR1, CDR2 and CDR3 sequences in each binding unit are present in a human VH or a human VL framework.

117. The bispecific antibody of claim 115, wherein the two binding units that bind to PD-L1 each comprise a heavy chain variable region comprising a sequence having at least 95% sequence identity to SEQ ID NO: 25.

118. The bispecific antibody of claim 117, wherein the two binding units that bind to PD-L1 each comprise a heavy chain variable region comprising SEQ ID NO: 25.

119. The bispecific antibody of claim 115, wherein the two binding units that bind to PD-L1 each comprise a light chain variable region comprising a sequence having at least 95% sequence identity to SEQ ID NO: 27.

120. The bispecific antibody of claim 119, wherein the two binding units that bind to PD-L1 each comprise a light chain variable region comprising SEQ ID NO: 27.

121. The bispecific antibody of claim 115, wherein the two binding units that bind to 4-1BB each comprise a heavy chain variable region comprising a sequence having at least 95% sequence identity to SEQ ID NO: 45.

122. The bispecific antibody of claim 121, wherein the two binding units that bind to 4-1BB each comprise a heavy chain variable region comprising SEQ ID NO: 45.

123. The bispecific antibody of claim 115, wherein the two binding units that bind to 4-1BB each comprise a light chain variable region comprising a sequence having at least 95% sequence identity to SEQ ID NO: 47.

124. The bispecific antibody of claim 123, wherein the two binding units that bind to 4-1BB each comprise a light chain variable region comprising SEQ ID NO: 47.

125. The bispecific antibody of claim 115, further comprising a heavy chain constant region sequence that comprises a CH1 domain, a hinge region sequence, a CH2 domain, and a CH3 domain, wherein the heavy chain constant region sequence comprises an L234A mutation, an L235A mutation, a G237A mutation, or any combination thereof.

126. The bispecific antibody of claim 115, further comprising a light chain constant region sequence, wherein the light chain constant region sequence comprises a human lambda light chain constant region sequence.

127. The bispecific antibody of claim 115, wherein, in each of the binding units that bind to 4-1BB, the heavy chain variable region and the light chain variable region are connected by a G₄S linker comprising SEQ ID NO: 36, SEQ ID NO: 37, or SEQ ID NO: 38.

128. The bispecific antibody of claim 115, wherein each of the second binding units is connected to a C-terminus of the heavy chain constant region sequence by a G₄S linker sequence comprising SEQ ID NO: 36, SEQ ID NO: 37, or SEQ ID NO: 38.

129. A bispecific antibody that binds to PD-Ll and 4-1BB, comprising:
(a) a first light chain polypeptide comprising the sequence of SEQ ID NO: 44;
(b) a first heavy chain polypeptide comprising the sequence of SEQ ID NO: 42;
(c) a second light chain polypeptide comprising the sequence of SEQ ID NO: 44; and
(d) a second heavy chain polypeptide comprising the sequence of SEQ ID NO: 42.

130. A pharmaceutical composition comprising an antibody of claim 115.

131. A method for the treatment of a disorder characterized by expression of PD-L1, comprising administering to a subject with said disorder the antibody of claim 115.

132. The method of claim 131, wherein the disorder is cancer.

133. The bispecific antibody of claim 115, wherein the Fc region comprises SEQ ID NO: 93.
